SEQUENCE LISTING (43 sequences)

SEQ ID NO: 1 - DNA, 1833 bp, Vibrio campbellii
  source 1..1833: mol_type = genomic DNA
  misc_feature 1..1833: note = nucleotide sequence encoding glmS
atgtgtggaatcgtaggtgcagtagcacaacgagacgttgcagaaattttggttgaaggcctacgccgcttggaataccgcggttatgactctgcgggtgtcgcgattgttgatggcgaaaccaacctaacgcgtattcgtcgtctaggtaaggtacaagagttggctgacgcagtagaccaagcgaaagtcgttggtggtacgggtatcgcacacactcgttgggctactcacggtgagccatcagaagcaaacgcgcacccacacatgtctggtgacatcgcggtggtacacaacggtattatcgaaaatcacgaagagttacgtgaactgcttcaatctcgcggttacgttttcgaatctcaaacagatactgaagttatcgcacacatggttgagtgggaacttcgcacttcagaaacgctactagaagcggtacagaagacagcaaaacaactggatggcgcatacggcacagtagcgctggatcgtaaagatccttcacgtattgtagtagcacgttcaggtagcccaatcgttattggttttggtgtgggtgaaaacttccttgcctctgaccaacttgcactgcttaacgtgacacgtcgcttcatgtatctagaagaaggtgatgttgcagagatcactcgtcgcgtagtgactgtatttgatgcagctggtgagcgtgtagagcgtgaaattatcgaatcaaacgctgaacatgatgcgggtgataaaggtcaataccgtcacttcatgcagaaagaaatttacgagcaaccaacagcgctgatcaacacaatggaaggtcgtatcacggctgattctgttgtcactgaagcaatcggtgtaaacgcggcagaaatcctaagcaaagtagagcacgtacagatcgtagcttgtggtacgtcttacaacgcaggtatgacagcacgttattggttcgaagacattgcaggtgtcagttgtgacgtagaaattgcgtctgaattccgttaccgcaagttcgtgacgcgtccaaacagcctgttgatcacgctttctcagtctggtgaaactgccgatacgctagcagcacttcgcttagcaaaagagaaaggttacatggcggcaatgaccatctgtaacgtcgcgggctcttctttggttcgtgagtctgactttgcgtttatgactcgtgcaggtgtagaaatcggtgtggcatcaaccaaagccttcacgactcagctctctgcgctactaatgctagtgacagcgctgggtaagcaacaaaaccgcatcagcaaagagaaagaaaaagagatcgtagaagcgcttcacgctctgccgcagcaaatcaacgcagcgctgtctttcgagaaagaaatcgaagcactggcaaccgattttgccgataagcaccatacactattcttgggtcgtggtgagttctacccaatcgcgatggaagcgtctctaaagctgaaagagatctcttacatccacgcagaagcttacgcggcgggtgaactgaagcatggtcctctagcgttgatcgatgcagatatgccagtggttgttgttgcaccaagcaacgatcttctagaaaaactgaaatcaaacgttgaagaagtacgtgcgcgtggtggcctgctttacgtattcgcagatgctgacgcaggttttgaaggtgacgagaccatgaagatcatcactatgccacacgttagtgaaattacggcggcgatctactacaccatcccaatgcagctgctgtcttactacgtagcattgatcaaaggtacggacgtagaccaacctcgtaacctagcgaaagcggtaaccgtagagtaa

SEQ ID NO: 2 - DNA, 852 bp, Vibrio campbellii
  source 1..852: mol_type = genomic DNA
  misc_feature 1..852: note = nucleotide sequence encoding thyA
gtgaaacagtatttagatttgtgtcagcgtatcgttgaccaaggcacttgggttgaaaacgaacgtacaggcaaacgctgcctaacggtgatcaacgcagacttgacctacgatgttgctaacaaccaattcccattagtaacgacacgtaagagcttttggaaagcggcagttgctgagctacttggttatatccgtggctacgacaacgctgaagatttccgtaagttgggtaccaaaacatgggatgcgaacgcaaacctaaatgaagcatggttgaacaacccattccgtaagggcgaagacgacatgggacgagtatacggcgttcaaggtcgttcatgggcgaagccagatggcggtcatatcgaccagttgcgtaagattgtggatgatttgacgcgcgatgttgatgaccgtggtgaaatcctaaacttctacaacccgggtgagttccatatagggtgtttgcgtccatgtatgtacagccaccacttctcactgttaggtgacacgctatacctaaacagcacgcagcgctcttgtgacgttccgctgggtctgaacttcaatatggttcaagtgtatgtgttcttggcaatcatggcgcaaatcacaggtaagaaacctggtcaggcttaccataagattgttaacgcacacatctacgaagatcagcttgcgccaatgcgtgacatccagttgaagcgtgagccactaaaagcggcaaccttccacattaacccagaaatcaaatctctagaagatttagaaacatgggtaaccctagatgacttctgggtagaaggttacgagcatcacgatccaattcagtaccctttctcagtttaa

SEQ ID NO: 3 - DNA, 2073 bp, Vibrio campbellii
  source 1..2073: mol_type = genomic DNA
  misc_feature 1..2073: note = nucleotide sequence encoding cqsS
gtgctaggctcagtagataaagtaaatatggacgcgattcgcaaagtatatcagtacgcagaacccaaccttagcctcgtaggatggatgggcttcataggctttcctatttactacatcgtttgggagttcatgttcccgcagccatacgaaaacctaccgcttcgtattctttgctcagtcttattcttcggcatcatttatcgtaatcgtacgccatttgagtggcgtggatttttacccgcctattatcaagtggtcacaacgctctgcttaccttgcttcttcttttacatgttgctgatgaacaattggtcgaatgtttgggtgatgtcgtttatgtcggcgatttttttgcacatactgctcgtgcatatcacatcggtgatgtttgttcagacatttgtgggaattggtcttgcgacgttttttgcttgggtagcacaggggtttcacctcgaactcaccatggattggacgcacgtaccgatcttcttgtttatttatctctttggtaacttgttctacttccgaaaccaggtagaacatgaagcaaaagtctcaatcgcaaagtcttttggggcgggtatcgcccatgaaatgcgaaacccactcagcggtttattaacttctatcgatgtcattcaatccgtgttgccaaatccgaaagagggcaagaaagagcagtacacgttaagtgatgaagatgtgacactgttacgagaagtcagcagtgacgcaatgaagatcattcattccggaaacgaaaccattgatttgcttcttacttcaattgatgagaaccgtgtttctcgctcgacatttaaaaaacactctgcacaatctgtcgttgagagtgcgattgaaagtttcagttacaaacgatcgacagaccgttttgctatctccttggatgtaagaagtgagttcgatttcttgggcagcgatacattgcttaagtatgtgatgtacaacttgttcaagaacgccttccaccaccgtagttcagaagacttccacatacacgtcaccatgtacagtgatgaatttgccaaccaaattgtggtgactgataacggttcgggcattgcgccagaagttcttcaaagtattttccaagatttctacactacgggtaagtcaggaaactacggccttggactgcctttctgtaagaaagtgatgcgttcatttggcggtgatattcgttgccaatcagaagtcggcgagtggtctcagttcacgatgactttccctacgattggctcaagcgcagtcaaagagattaagagtgagctaaccaagctaaaaacgattttgtttgtgagtgagcaaaacatcttagtttcgaaagtgacggacattgctcgttttatgagattcgaactcacggttctcgatgttcctgcggttcttaaaaataaagaatacgagttcgagtttgatttgatcctgatcgacatggagagtttagacgccagtggcagccatatagataaggttgagtcgttactgtcgttcactgaagctcgcattgtttacatgtttgagcaccatccaattcaaagagcgagaagcgtttcttttgaacctatttgggtagagacacaagcctggcttctgaatactcgagcgaccatcgaccgtttactgtatgacgctaattatgttgttccatcgatgccagcgaaaccgctcgattcaacaaacaagcgaaccattatggtggttgacgataatgagtctttacgcaagtttacggccatgctgcttgagaagcagggttttgaagtgatccaaactgaggatgggctgcaggcaatcaacgcattgaatgagaacaatgttgacttaatcctaatggacatcgaaatgccggtgatggatggtgtagaagcatctcgtcaaatcagaggttcaaataaggcctacgcttcggtacctattattgcgcatactggtgatagctcaccgataacactggacaagattggttcatctggcatgtcggatttcatcgttaaacctgcggataagaacaggttgttcgacaagattgcgaactggatttag

SEQ ID NO: 4 - DNA, 3677 bp, Vibrio campbellii
  source 1..3677: mol_type = genomic DNA
  misc_feature 1..3677: note = nucleotide sequence encoding luxPQ
atgaagaaagcgttactattttccctgatttctatggtcggtttttctccagcgtctcaagcaacacaagttttgaatgggtactggggttatcaagagtttttggacgagtttcccgagcaacgaaatctgaccaatgctttatcagaagcagtacgagcacagccggtcccactgtctaaaccgacacaacgcccgattaaaatatcagtggtttacccaggacagcaagtttcagattactgggtaagaaatattgcatcattcgaaaaacgtttgtataagttgaatattaactaccaactgaaccaagtgtttactcgtccaaatgctgatatcaagcaacaaagcttgtcattaatggaagcgctcaagagcaaatcggattacttgattttcacgcttgatacgacaagacaccgtaaatttgttgagcacgttttggactcaacgaacaccaaattgatcttgcaaaatatcactacaccagtccgtgagtgggacaaacatcaaccgtttttatatgtcggatttgaccacgcagaaggcagtcgtgaattagcaacagaattcggaaagttcttcccaaaacacacatattacagtgtgctctacttttctgaaggttatattagcgatgtgagaggtgatacttttattcaccaagtaaaccgtgataataactttgagctacaatcagcgtattacacgaaggcaaccaagcaatccggctatgatgctgcgaaagcgagtttagcaaaacatccagatgttgattttatctatgcatgttcgaccgacgtagcattaggtgcagtagacgcactggctgagttgggacgtgaagatattatgatcaatggctggggtggaggctctgctgagttagacgctatccagaagggtgatttagacatcaccgtcatgcgtatgaatgatgacactggcatagccatggcagaagcgattaagtgggacttggaagataaaccagttccgaccgtatactcaggtgactttgaaatcgtaacaaaggcagattcaccggagagaatcgaagcgctgaaaaagcgcgcatttagatattcagataattgatgacaacaacgcgatcaaacattaaaaagcgtcgctcgctggcgacgctcataacaaagatcatcattttagttcttgccccaattattctggggattttcattcagagctattacttctccaagcaaatcatttggcaagaagtagaccgaaccaaacagcaaacctctgcactgatccacaatatatttgatagccactttgcggcgatccagatacatcatgacagtaattccaagagcgaagtcattcgtgatttctacactgatcgcgacacggatgtgctcaactttttcttcctcagtatcgaccaaagcgatccgtcgcacacaccagaattccgttttctaacggaccacaaaggcatcatttgggacgatggaaatgcgcatttctatggtgtgaacgaccttatccttgatagccttgccaatcgggtcagtttcagtaacaactggtattacattaatgtcatgacctccattggttctagacacatgctcgtgcgccgtgtgccgatcctagacccttcaacaggagaggtgcttggtttctcatttaatgccgtcgtcttagacaacaacttcgctttgatggaaaagctcaagagtgaaagtaacgtcgacaatgtggtgctggttgctaatagcgttcctttagcaaactctttgattggtgatgagccatataacgttgctgatgtattgcagcgtaaaagttcagacaaaagactcgataagctgttggtaatagaaacgccaatcgtcgtaaatgcagtgactaccgagctttgcttgttgacggtacaagacaatcagagtgtggtgacattacaaatccaacatattctagccatgcttgcatcgatcatcggtatgatcatgattgccttaatgagtagggaatggattgagagtaaagtttcggcgcagttagaatctttgatgtcttacacccgctctgctcgtgaggaaaaagggtttgaacgatttggcggttcggatattgaagagtttgatcacatcggttcaacccttgaaagtacattcgaagagcttgaagcgcagaagaagtcgttccgagatctgtttaattttgccttatcacccatcatggtttggtctgaagagagtgtcctgattcagatgaaccctgccgcgcgcaaagaattagtgatcgaagacgatcatgaaatcatgcatccggtcttccaaggctttaaagagaaattgaccccacacctcaaaatggcggctcaaggtgcgacgttgactggggtgaacgtgcctattggtaataagatctaccgatggaacttgtcgccaattcgtgttgatggcgatatcagtggcattattgtgcaaggccaagacattacaacacttatcgaagccgagaagcagagtaacattgcgcgtagagaagcagaaaaatcggcgcaagcacgtgcagacttccttgctaaaatgagccatgaaattcgtacgccaatcaacggcattttaggtgtcgcccaattattgaaagattctgtcgatacacaagagcagaagaatcaaatcgacgtcctgtgccacagtggcgagcacttgcttgcagtactgaacgatattctcgatttctcaaagatagagcagggcaagttcaatattcagaaacacccgttctccttcaccgataccatgcgtacattggaaaatatttatcgtccgatttgcacaaataagggggtggagttggtcatcgagaatgagcttgacccgaatgttgaaatcttcaccgatcaagtccgcttgaatcagattctatttaacttagtgagtaatgccgttaagttcacgccgattggctcgattcgactgcacgcagaacttgaacaattctatggtgcggagaacagcgtgttagttgtggaactgactgatactggcatcggcattgaaagcgataagctcgaccaaatgttcgaaccttttgtgcaagaagagtcgacaaccacacgcgaatatggcggtagcggcctaggtttgaccatcgttaagaacctagtcgatatgttagaaggtgatgttcaggtccgcagtagcaaggggggggggacaacatttgttataacacttccagtaaaagatcgtgagcgtgtcttaaggcctctggaggtcagtcaacgtatcaagccggaagccttgtttgatgaaagtttaaaagtgctactggtggaagataaccataccaatgcgtttatccttcaggctttctgtaagaagtataaaatgcaggtggattgggcgaaagatgggctggacgcgatggagctcctttctgataccacctacgatctgatcctcatggataaccaattaccccaccttggtggtattgagaccacgcacgagattcgccagaacttgaggcttggaacgccaatttacgcgtgtacagcagacaccgcgaaagaaaccagtgatgcgtttatggcggcaggtgcaaactatgtcatgctgaagccaattaaagagaatgcgttacatgaggcgtttgtcgatttcaaacaacgtttcttggtagaaagaacctaa

SEQ ID NO: 5 - DNA, 2550 bp, Vibrio campbellii
  source 1..2550: mol_type = genomic DNA
  misc_feature 1..2550: note = nucleotide sequence encoding luxN
atgtttgattttagcctagaggctatcgtctacgccaaagccattacgctcctcgcgaccgtggcagtcgtcatgatgtggctcttctattactgctatcgacttaagcaaaaaaatgaagtgatctttggtactcaccacgcagcttatattgcgtactcggtgtgtatcattgcgtggatcagcagcaatgcttatttccataccgatttactgcccgagctcggagcttcggctggcatgttcatggccaagtttgccaacctcgcttccttcttcgcatttgcttttgcctattatttctcatgccagttggcggcggagcaacgcaaaggcaaagtacaccgatggcagcaaggtatctttgtttcattaactgtgtactcactgtttatcaatttgcgtcccggtttgaccgtagagcacgtcgatattgtcggcccaagtcaattcatcatcgagtttggccctcatacctcttatttctttatcggcctcgttagctttgttgttctgactctcgtcaatcttgtcgccatgcgaacaaacagcagtaagctgactctagccaagaccaactacatgatcgccggtatcttagtgtttatgctttcgacggcagtcattcatcttggtatgacttactttatgggagatttctcattgacttggttaccaccagccttgtcgataagtgaaatgctctttgtcggctatgcactgctgacctcccgcttctacagcgtaaagtacattgcttacctcgctctcagcgtgctgctggtctgtgctattttcgttctgcctttaggcgcaatattcatcccacttacggaaagtaatcagtggcttatcgccatacctatctgcgcccttattggtattacttggcagcttttgtataaaaagaccagccgatatgcatcgttcttgatatatggcgacaagaaaacaccagtacagcaaatcttgtcacttgaagaggattttaagctttctattgatgatgcgatgcgccgtctgggtaagctgctgcaaattccaaatgacaaactacgcctcgtaaccagtaactacaacgaaaccttttacgaagagtacctttcatcaaaccgctcggtgttggtgtttgatgaactctctgaagagctcgaatacaaggtatcggcaaagcgttcaatgaaggcgctgtatgacaaaatgagctcaaacaacaccgctttggttatgccgctgtttgggcaaggtaaatcggttacgcacttattgatttctccccacaagagcaacaaccaaatgttctcgaacgaggaaatctcagcagttcaaaccttgcttactcgagtacaaagtaccattgaagcggatcgccgtattcgacaaagccgcgcactagctaactctattgctcacgaaatgcgtaaccctcttgctcaagttcagttgcaatttgaagcattgaagcagcatattgagaatcatgcgcccgttgaacagatcacactagatattgaaaatggccaagccgcaattcagcgcggtcgccaactcatcgatatcattttgcgagaagtgagtgacagctcgccagagcacgaacctatcgctatgacctcaattcacaaagccgtcgaccaagctgtcagccattacggttttgaaaatgagaagatcatcgaaaggattcgtctgccacagcacactgattttgtggcaaaactcaacgagaccttatttaactttgtcattttcaatctgattcgtaacgcaatttactattttgactcgtatccggacagtcaaattgagatcagcacgaaaacgggcccatatgaaaatacgttgattttccgcgacactggcccaggtatcgatgaaaccatctctcacaagatctttgacgactttttctcttaccaaaagagcggtggcagcggcttaggtttgggctactgccagcgcgtaatgcgttcttttggcggcagaattgagtgtaagtctaaacttggcacattcacggaatttcatttgtacttccctgtcgtcccgaatgcaccaaaagcagacacattacgcacgccttacttcaacgattggaagcaaaataaacgaagtaatgaacataaagtcgcgcccaacgtacaaataaataaccaatcaccaacagtgcttatcgtcgacgataaagaggtccaacgggcgctcgttcagatgtatttgaaccaacttggcgtgaatagcttacaagcaaacaacggtgaaaatgccgtcgaagtgtttaaggcaaaccacgtagatttgattctgatggatgtacaaatgcctgttatgaatgggtttgatgcaagccaaaggatcaaagagctgtctcctcaaaccccaattgtcgctttatcaggggagtctggagaacgcgaattagacatgatcaataagctaatggatggtaggctagaaaagccaacaacgttaaacgccttacgtcatgttttaggaaactggttgaacaaaaataccgcatcgagcgcttgtgaagctgagagagaatag

SEQ ID NO: 6 - DNA, 1200 bp, Vibrio campbellii
  source 1..1200: mol_type = genomic DNA
  misc_feature 1..1200: note = nucleotide sequence encoding luxM
atgaaattaatgttgtcattaggctcgctgtcggctaacagtttgcctatcgaaaaaaaacagcaagtgctgatcgatcttgtgatccgcacttatcagtcacacgagcgaacggagctttttaaggcaattacggaatatcgtaaaaaccaactgatcgctctctttcctgaacatgctaacaaaagctattccattatctttgagctgatggactatcgtgaccttattgaacgatatcctagcaccctgtccgaagaagctactttgcttgaaaaagtcgttggtcagtgctttatgcattggttggacttctggtgtgagtgcgagattgccgctattaaagcgaagtttccgttgaaggaaaacgaactaccagctccacaacttctctttgaagacagtgcttactatggtgctctagttgagcgagtagaagacactcaacttatggtacaaatcccaagccacccgcaggcaatgccacttagtgatgcgatcacgttgagtaatcttgagctttttatacaaggtgaaaagtggtacgagatgctgtcgctattgtctttgtctcaagtaggtaagcacttcatcgtactgaaacacccagttcaggattcttgcccgactctggtggcttccgcgcttattcagaattggtctgttcgcgatacttggctcagctacgcgccacagtttagtaacgagcagtggaattactgctttcctagttatggctactctgaatttacgcgtttacagctatttacaccctcaacgctgtcaaaatgttactctctcccagaatttgataacgaattcaagttacaattgtctgatacacaagccgtttgcgaagtgttgcgcctgaccgtaagtggaaacgctcaacaaaaactctacttcctctacctcgctcagaaagagttgatgagtgtattgcatcaggcaggttacaaaattggcttcactattatcgagcaacctttcatgctcaacttttatcgagctatcgatgcgaaggcgtatttccattcgggatactgcgacttaaatgacgatggcaaacagacttaccgtggattctggaattttgaaatgatggtcaaagcgttcagtaacatcgactttcgtggttataaacgtgctgttcgagcgagcagaaaacgcggttcgttagaaagggatgagcatgtttga

SEQ ID NO: 7 - DNA, 975 bp, Vibrio campbellii
  source 1..975: mol_type = genomic DNA
  misc_feature 1..975: note = nucleotide sequence encoding luxB
atgaaatttggattattcttcctcaattttatgaattcaaagcgttcttctgatcaagtcatcgaagaaatgttagataccgcacattacgtagatcagttgaagtttgacacgttggctgtttacgaaaaccatttctcgaacaatggtgtggttggtgccccattaacagtggctggttttttacttggtatgacaaagaacgccaaagtggcttcgttgaatcacgttattactacacatcatccagtacgtgtggcggaagaagcgtgtctactcgaccaaatgagtgaaggccgttttgtctttggctttagtgattgtgaaaagagtgcagatatgcgcttctttaatcgaccaacggattctcagtttcagttgttcagtgagtgtcacaagatcatcaatgatgcattcactactgggtactgccatccaaacaatgatttttatagttttcctaaaatctctgttaacccacacgcgtacactgaaggcggtcctgcgcaatttgtgaatgcgacgagcaaagaagtggttgaatgggcggctaagttagggcttccactcgtgtttaaatgggacgactcaaacgcgcaaagaaaagaatacgccggtttgtaccacgaagttgctcaggcacatggtgtcgatgttagtcaggttcgacacaagctgacgctgctggtcaaccaaaatgtagatggtgaagcagcaagggcagaagcacgcgtgtatttggaagagtttgtccgtgaatcttacccaaataccgactttgagcaaaaaatggtagagctgttgtcagaaaatgccatcggtacttatgaagaaagcactcaggcagcgcgagttgcgattgagtgttgtggtgccgcggacctattgatgtcttttgagtcgatggaagataaagcgcagcaaagagcggttattgatgtggtaaacgccaacatcgtcaaataccactcgtaa

SEQ ID NO: 8 - DNA, 9427 bp, Vibrio campbellii
  source 1..9427: mol_type = genomic DNA
  misc_feature 1..9427: note = nucleotide sequence encoding the deleted region from Phi-HAP-1-like bacteriophage region
atggtgacgtattggaaaaaggaagactcgagcaaaataaaaaggcatcaagttgctagtgctcagattgaacgaaagaaatctgggaccagagagggggttggtatcgcactggatgcgattggttacggttgtgaaattaagtctcgttatgagagtccagatttgccaccgtacacgcttgatgttgttgcttggaaaacagattcgtcttcggttaataaagaactaatcgagaatcttattctcaagttagatgacatcaaatccacaagagacaccatagaactatctttgctctttggtgtggaaactgaaatagcattgtctggtgcaaaatcgccaccgacaaatgtcagttatacagacgcccctgcagtattgtggcccatgcctgatgcggatgtcgcatttggtattgtaggtggcgcctgtcttcccattagtgttaaccagttgaccgttaaggcaaccgtcataccagacgaaccgatagcgagtgctgcgcctgctgcaggcagctatagcctttctgtgtcacctattaccgtgagagcaattacatgagtgacgaaggattaaagttagtcatcacccgacgaggtttgggtgcggcaattaatgccaaagataaaggcataaaagcggcattaaaatgggtgagtgttggtgatgccgcttataccccaaataatgagcagcaagttctacaaaatgagctggctcgagtagagttcggtgaatataaagatacaagcggaagcagtatccaagccgctgcgaagttttctggtccagaagcctatgcaattcgagaaataggtttttggttagatgatggcactttgttcggtgtgatatctgaaccaaataggacgcttaattataaagcggcgggtggtcactgtattcagccatttacgcttaacctttctgctcttcctagcgattcgatcacggttatcgttggtactgaaaatctgaatatcttaattgatacagagtttgccattcttgcaaaaagccaagtagacacaatgcaccttgctatcaagcacgagtttcgtttgcttgagttagaaaaatcgaaatcataaggagctcattttatgagtacacaagaaattggcgagttaattggctcggttaataacctgactcaaaccgttgctggtaagatgggggatattgacaatcgaatggatcaagctgaatcagaatttgatcagtttatggttcaagcccgtttggaaaatgctatttttcgccagagtaaaaatcaatttggcaatttgacaggtacgacattagattattttcagcataattcacaatatacgattgaagttagtctatatcgtgaaattaagactggtgttgattggggttcaagagatcttgaagagcaagagatcatgaaggccatgggaatgagcggttggaaatattttcaacctaaaattcgtgtgatgaagatggtttggagtggatttaatgcatctgcacattcatcttactccctatttccaagacccatcggtaatggaacgggaacgttaactattgctagttacgctaagttaataagcggttctattcgtggttggtggttggaaggtgtcaatgactcatggggactatgtggtgttcaccataatgggcgcccgggtcgatacattcatgctcatccatatgtcgccacgccatcgggggaaatcctgtttatttggcccggtacagtttcaggccatgtcccactcgaacgtagttcacctaagtggggctattggccttcaatgtatggtgaaaacccttatgatgcacaaccgggagcttaattatggaacatgaagaatttgattactctaacgatgtgttgagcttaaaagatattaatgagcgttgcgaagaacatatcacttatttttatccaattggtaagcagcttacgatagagcgggttgggacggaggaagaaaagaacctcatgtattcatttattgatgcttgtcgtgcttgggccaattccgaacatccaaaggctaaagatttatctgtaattaaacctcaataagttaaccgtccaatcaggcggtttttttatatctaaatttcatcattagccctgccttagcgcagggcttttttttaggagccgttatgcaccaagcaaaaccgacgtctaaggcgaagcgcgaaacctacatcgtagagcttccgttcccttggcataaaggtcattggacaatcaaagaccaagagctacaccttcttccacaggaagctaaagcgctactcaccaacggtcgtatttcaaagaaagcgactgctaataaaaccaccgttaagagcaagtaatttatggaaatcactcaatttgagcacaatggtatttccattgtgaagaaagccttgcctgcccctatgggacctttgggtaagcatatcgtttgttgggtaggaacagcgccagacaagcatgccgatgttccttttaatatgccatttcgcgtagctaatacgactgatgccgctaagctggataccactggcgaagaagatggttggttgtggtatgcggtgacggaaactttgaagaaggttcaagtgcctcagtatgtcatcgtggtacctgaaggtgcggatgcaacgaaaacgcttaacaacgtgatcggcggtattgatgctgctactggtcagcgtacaggtattgcggctatttcgctatgtacggaatccccgaccattatcgcggcaccaggttacacccatcaaaaaccagtttctgatgcgcttgtagcggaagccttgaaactgatggcgttcccacatttggatggattgtctaccagtacgaaagcagtcattgatcactcgttgacattggcaactcaagatacgggttatgacattgcgaccatgattgacccacagccaagcatttattccaaggcggctggtgtgaatgtttacgtgccgccatcggtgctttcggttggctgtcatgctggcgtggcattgcatgaatcccctggtaacaaaggcacctacgctcaaggtgtacaacgtgacattacctacgacattctcgataccactaccgagggtgatctactgaaccgtcacggtatttgctacttcggtaaaacggacttaggcggtatttcactgctgggtaaccgcacactttcaggtcgttttattaatcaggtgtgtttggagcaagccatttgtcgcaagctgaaaaagtctgcgcaaaaggtgatggcggaaaatctcgataaatcgttcatggaacaagaaatttccaaactgaacgcatggggggctcagctaaaagcgaatgaagaagtgatcggtatggaagtatcgcttcatccagagcttaatacggctgaaacctaccgtaacggtacctggtacatcgcaattcgttacgctggcttcccaccaaatgagcacatggtttaccacctcattgaggatatcggcattatcgacaactttatctcggagatcttgtaatggcaggtcaacgcacattagtaatggccgaagttgtgatcgaaggtcacaagttcttcggtgaattagaagaacatacgccaccagtggtagagcgtaaaacagaagatgccaaaggtgggcgctatgcgtccacgaagaagcgtgttggcgtggtggtaggtaatgccacccttaaactgaaaggtgccacggtagaagcgctgcaggcgtatggtgtggaagaagacacacgcgttcagattgacgttaaagcatcgtaccaagatgaagacggcaatacatttgcagagcattgctcatacacgggcgacattaataaagtggatgatggttcgcgagtatcgcaagctatttctgaaaccacgattgaaatttcgcctcgtatctacaagcacacagaagatggtaagactcgctataacatcaaccttgatactcaagaaatcgacttgggtaaaggtgacattatggcgaaacatcgcagcaatctcggtcgcggctaattgtgtccgtggacccattttaaatttctagctagttggctcctggaagggagccttttttcggagaaaattcatgactctgcaaattgaaaacaaaaagaaaaaagaactaaagcttgccccaattacgcaccaccttcttcacccaatcaccgttgatgatactgaaattaccgagctaacgtttgaacctttgacgttcagcatgtatagcggtgtcgatgttgaacaagacgatcaggaagttgttgttgaagagttcacgctggcgttaacgggtatttcagaaaaggcgtttaataagctatcttcaccagattttaactcggtagaaaaaatcattttccgcttggtcacgaagaacgcaccagattggttaccaggcattgaaattggtagtgagaaaatcaaactggctctgccaatcgtcgctgatgatggtcgcacgatcactgaaatagaattaaaagtgccatcggtgaaaacgcgcaagatttacaaggctcagccaacggataaggaaaagcaggcgtttattactcatgcttgttcaggtttaagcgaagtcgaagtaggcaagttgttcatgccggattggaatcaactgcaggccaatattgataattttttaaaccagcaggcaggcttctttcagacaaagatatagaagcgctctgtgattatctgccgctcgtttatccctataacgatgagcaaataggcgcactaccagcagacgtggcagtgcgccgtttttatttggcaattgaaaggttgaagcaacaatgagtaatcaatctaagtatcaagttgccattgcagcgattgaccaatactccgcacctctcaaggcggcaagcgcatcgtttgactcactgactgaggatgttaaagctcagtcggcagagatcaaaaagctcaatgccagtgccgccagccttaactcttacgcgagcattaagcgtgatttaactgaaacgtcagctcaaatgggctcggctaaagtgaaagcggagcagttggctgtagccacaaaggagcttagtctccaaacgaaaggctacgagtcagcagtttctagttctcaaagtaagcttcaaaaccttgaagctcaaatgagggcaacggaacacccgagtaaagctctgcgaacagccattaaggaagctcgcaaagaagttaaactcaatacagccaatcttaacgaacatcagctgcagctgagtaaaacacaggctgcttatgataaatcacgtcatgaagtttcccagctcaccaagcactacgatgttcagagaacgaagcttcatggtctgaaatccaagttaaatgagtctggtattcaggcgcataagtttggtgaagcacaacgtcagatcaagcgtgatattacggctgcgaacgctgcactggataagcagaaagccaagctgaaatcggtgcaggctgcatctgcaaagattgagtcgaataagatggctcgctctgaattagggggtgaggcgttagatttggcaatgaaaggtgctgttctggcagtgccgattaagtttgctgtcgatttcgaatcagcgtttgctgatgtgaaaaaagcggtgaatgatgccagtgatgaagagctggaggtgatgaagaaaaggatcgttattgaggcgcctaaattgggtgttactcaagaaggtctttctgcgattattgccgagggtgctcgtaacggcatcgcaaaagatgagttgtttaactttgctgagtcagcagcaaagatgtctgttgcctttgatatggccgccgatgaagctggcgcgtcaatgatgaaatggcgcacgacgatgaatcttagtcaggatcaagcggttaatctggcgaatgcggtgaactatgttggtgacaacatggcaaccacggcaaaagacatcaccgaagtgctggttcgtcagggtgcagtgatcaccaatgcaggtcttgatgaggttcaagcggcctcactatcagcagcagtgttgtccggttcagcaagtacagaaatcgcggctaccgcgacaaagaatttgttgctgagtttaacggctggcgattcagcgagtggtggtcagaaagatgcgttaatgaccttgggatttgacccagccgatttagcgcgagacatgcaggaaaacgcacctaagacggtagagcaggtgctcttggcaattaaagaccaagatgccgacgttcaaaccgccttgatgaagaacctattcggttctgagtctatcggttcaatcgcgccacttttgcaaaaccttgataacttccgtaaagcattcaagttagtagagaaagataccaactttgcaggctctatgcagaacgagtttgaggttcagtctgctactgccatgcggaaaatatcagcctttactgcttccataaccggactgttcaccgttttaggtgaaagcatgctgcctgttgtaggtgatgtgcttgataccataacgccagcagtcacatggttaacagaagcagcacaatcggcaccaggtgcaactgctgcattaatggctattcctgctgccttggtggctgtaaaaggtgcagcattagcttttaaagcaggtaagctgttacttggtcagggtaagaactatgccgatttaggtaaagctaagcttggcattggtttggatggtactgcggaatcggctcagaaagcgacgtcacgcctttctcgattgaatcagacattggataaccttggtaacaatgggggccgtggtcgcaatcgttatcaacgtgatggtgcatctacttccagtcgtcgtaaatctcgtcctaaaagagcaagattatcgcgtcgtggtggtaagtttgggcgtctgcttgattttggtagtcgattaactgacttccttcctatgggtgcgccagcacctgcctttgcggcatcctctaatgggtctgcaaaagggaaatggggcaagcgagctgctgttcttggtggtggtactgcgttatcaatgcttacctcaagtgctaatgcggctgatttggctctgatgggagctgatgcggcatctgtggcatctgtggcaggtgatgtggcaggttctttgccgttaaaaggcatgatggcgggtattgcaggcacagcaggaaaactattcaaaccacttaacgtcatgctgcagggcgcggcattaacttctgcgatcaataacggtagcgctgaagaaatcggaggtactgcaggtgatatggcaggcggtttgggtggtgcggcattaggtgcaacgattggtaccgcgattttacctggtataggtacggtcgtaggtggtgctcttggtggtttagccggtggcgaacttggcgagtggctcgggacgaaagttgctggtctgttttctagtgatgaagaggaaactctgactgctaaagcggcaacgctggacaaagaaaaagacggattaactcagcctactagccagttgcaagctgatgctgtgctgaatggtcctgatggcggttcaagtttggcaatgttacccgaaccgacgagtgtgcttccgtcaccggatagtattgcgaagtcgctttctcagacaaatcaagataaccgcaaggtcgaaattaacttaactatcccaccatcctcaagcaatcctcaacaggacgaggatatgctgaatcgtttggtagctaagttgaaagatatgatgatggctgaaggcatgatgggttctggttcgttatcagtggctatggacggttctctttctgataggagtgatgtatgagacagcagctaattctgtctgtcccgtctaagaatgaagaagcctttgtatttggtgtcgcaagcggcaccgaatatgaatcgctttcgacgaccagccaaggtggttgggtaaatctggatattatggacggcaaagcggtaagccagaacactcatgatccccttgatggtaagacgataaaagggaagtggtttggcgcagagggtagagcttctatccgtcgtttgcgtgaaattaagcgtatgcgagcgcccgttctactcactgatgattatgggtacaacatgggcttatggaagatcatgtcactcaacgatgatgagaaagatgtgattgatgacggtacgcctatgattgtgagttttaccattacgtttgaggagtttgccaattgagcacagtcgcattaagtgtggttggtgatacggttggtgacgtcctgtatcgttattttcaggacgatggtgatgatttggagcgtcagttgtacgctctgaatcctcacctcaatcagttacccgttattcttccaactggaacacgaatcaagttgccaacggtcgaggctaaatcagagcctgagccagagagagtggttactatatgggattagcaactcggattattgcccgtacaaccggaccgggcagtaaagtcattaatcaataccttcaaacgtggcagctcactgatgtttctggtgaggaaactgatcagattaggctgaaagtggctgcgcctgatatggattcgctaccacctgagggtgttacgataggttttgatattggcattgcggatgatgagccagttcagtggttcagccgtggtaagttcaccattacacgaattacaccgcagttgtttccgcatgtatttactattgtcgcaacggcagcgccttttcaggtcaatgaccaaacggagtttaaacagcgccgttcgcaaacgtatggcggtacgttaggtagtattttccgtgaagtcgtcagtcgccatggtttgagtcctagggttgatcaagatctggatagcatagttgttgagcatattgaccaaacggacgagacagacatgtcatttttgacacgattagctagaaaatacgatgctgtggccaagcctgtagaatcgttctatgtgctggcacgacgaggtaaaatcaagagcctttccggtaagacggtagaaactgttgaaattgaattaccaaccaataaccagccaaccgagaaaagttttactaatgccagcttaaacatgcctagccgaaagctatttaaaggcatcaaagcagcttggtggaatgatggtactggtgaagaagttattgaaatggtcggtatgaagccattcaagaagcttactcaaacttatcagaatgcagatcaagcaaagcaggctgctcaggatgagttaagaaaggtcgagcgaaccggaacacagattcgtctcgatcttcctgcaaaccctcgtttctatgctgagggtatagtaaaaacctcaagtcaattcccactttacatgcaaggggagtggtcactggatagagtcgtcatgaccggcggaaagcaaggcgcaagagcccagctcacagccacattcacacaaggt

SEQ ID NO: 9 - DNA, 10060 bp, Vibrio campbellii
  source 1..10060: mol_type = genomic DNA
  misc_feature 1..10060: note = nucleotide sequence encoding the deleted region from Kappa-like phage region
ctattccttttgtgggatttccttcaaacgtttaccctgaagagaggcaattacatcgtccacggtctcttggatcacgtcgttggtactcaagtcacgaagcgctttcagtccccaaataatggtgcgagatgcgaatcgctcaagcaccacttcccaagtgatttggagaaacagccctttcaagatttcgaataacgtcttgccgatgatccccgttaaaaagctcatgattgttctcccatcatggataaataagcgttgaggtaatccgcttctgtggctttacctgcggacgtgttccagtactgtttggcgtaacgcgctaaaccttcaaggtcatccgcttctggcaaggcttctgggaaccggatcaggttcaagcgcgccgtagccaccgcgaattgaggagaaatcaccatatagtccgcatcctgtttttcgacgggatcggcggtcagcggcacaaacatcgagagcgcgtccaacaagtgaggccgtgtttttcctagccattcaatcaaccaagcaaaggtggcgggctccatttgagtgaacccaagcgcaggacctcgcacctgcttggcgtacgtcaaggcaccggattcatgggcgacaatcatcactatcaggttgatggcggcttgggtattcagcttgccatgcccgcctgtgaccatgtctaattggtcgagtactggcttaatcacatgctcgataaagagcgttcctaatttcatcgtttcatcttctccaaatcgctttgacagtgagtgcagtactgacacccggtcatgtgttggcggcgagcttcggggatggggtcgccgcactcaccacattcctgcgcactctctcgtgaggcttttggcttagccctcgccaattggttggccaatgccacttctgtgagttgggcttcaatatcacaagcatggtcgataacatctggcatcctgactcctaattactggatcagatcttcggtttcatcggggcgtagatatggcacgccgttgattttcacaaagtctgggctggtcacttcgaacggcagcttatgcacgagcgcgctgcctccgttggtgtcggcgtcgagtaaatccgagatcttgatacgacaaccaaaggcttcgattttcagttcgtctttatcaatcttgccgtagaacagggcatcaaagtccggcataccacgccaggagccggcgcctctcgcggccttgctcaactgattaaattgctgcgtggtgagctccatctcgccactggcttcaacatcgccgtcgacataaccatccggcacgcccgaggttttattgacggcggaattgtccgtaatagagaggctgactttttgcgctttgagtttgtagtcgcccattgaaaaatgcatgttcttaccagaaatacgcatgctcatggattaagcctccgtgtcggctgggttagagagatcgagaccaatattgaccacgatgtgttttgggcaattgtgtgggcgcaccatcaaaccaatgttgactttggtttttgtcatccactgaatcgacacgtcttcttcgcgcggtgccataatttccccagggaacgggatccccccaatctcggtggtcttcgacatgtcacgcatgtctttgctgaagtaagttcgattaagctcgatactcggtggcgtcgaattaaggatgcgatcggcaatgcggcgaatcgctttaatacgtactcggcggttcaacttgtgaacggggcgaacgtattctaagaattgatagtctccgcctttggtttccagtgtggtggcgtcggtccagtacaccccttccatatcggcgtaccattgcggcaaggaataacgtgcattggccagcgctgaaatggtgctcatttccagtggttttccggcgctgtcgataggcatctcacccaggcctaaaacgctgcctgtggccactcgcattgggctatcggcgacggtgaccgcgcggtcacataagcgtcctcccaatacgccgatgttattgccattgagcataggtatcggtgtcacgaggttggcagatacaccattgattaatgccaataggctagtttcgtatttggcccaagtttgcttgctcttatcgatgccaggacacgctgctaaaaagaacacccaacggcccagcttggcggtcagttcggaggctttgctttgcatcgcttggaagtccgctttgctgcttaccacatcgacaatgcacacgccttcgaacgagtcggtacgattggcaatatcgacggcttcctgccacgttttaccttcagcgagaccaaacacggcgcctgtccagttctgttttccattaagctgagcggctttcaagttagcgcctagcacgtcatccgcgacgacatcgtcaaggttagtcatgttgttgacgcgtgtgactttgccttgcagctctgctttatcggtgcgcccgatatacagcaggtggcgttcaatctttgggatcccaccttgccctaaattgaggttgttgacctcgacctttccggttgccattagttatgtcctcgcttatttttgctttctggcttgctcaaaaatggtgatgagttggcgggtgacgtcacgctctttactgcctaatatctgacgttctgccaatggaatatcccaagcggtgacactcggctgattgctcagctctcggataatttgcccagcttggccatggctaacagtggccatcaataacttgagtgagggtttcttccttcccttgccactctttcttgcgaccgtgtagcccagttccctcatttttcgcgcttgccctttggtgcaaggcgcggagtagtcgggtgttccccaccgcttttgcatttggcgcttggtcatcttttgcttttgaccaagatgatgccgagctgcaatttttgcggtgagcttgttgctccaagtcaggtcgagcatgttggcgtttcgtacataaggggttaaccctttcgccatgcgccgcataactttgccgcgcttcttcccttttttgggttctaatgcttgcccgtgaatatctttttgttgctgaatgcgtttgcgtgtattggctttttcccaacggccaagggttttcagtatccaaactcgcttctttggtggcagtgctaacattgcaagcttttcttgcatgttgagcacatccctttgattcgcattaattgttggtttcattcaccaactccgcttcttctgcggtgtaaatctccacggcttgaactcgatatctcatgccacgccaagtgatcatgccggcttcatctgggaccaactcaatgggctccatcaactcaagttcaatactcacatcggccgcttcactgctgatcacatcgaccgaaagcgtggggtcttcgagctcttgctcgttgcgctcttcttcatgatcactgagccaacaagcaataagagctagtaagcagcgtgggtccagtaattggtgaggaaactcctcaaccgaaatcaccgcgttatatttccagtaacaggcgatatacccgccgttcccacggtcttcaccgcttggcactatcgagccgttttcttgccaagcatccattttgttctcaagcacattgctattgagatggctgacgatgtaagccgttaagtgctcaagtttggtttggttgtaaattgcactactcatatcacgccaatcccattcgcactgcggccaagcagttgagagacatctttgttactttgcgccagaaaacgtgccgcctgttgcggttcatctaacgccgcattgtccccttctttgcgcctatcttgcgtggcgaactcgggtaacaattccgagtgtgctcggccatacacagctcgtttatacaaggcggttttggcgacgcttagtgatggcggcattccctcgaccaacaggctcactaacttctcttgaatattgttggccgctatcgtgatggccacagctatagagtcgttatcaaacgtatgcggaatgcggcgcagttgacgaaactcatcgttcgttaaatcgggccagccttcaccggatatcacttggttactcgccttatccacttttccgccaaagcccatcatgtctccttattggaataagtgcgcctctggccactgagtcgacggaataagcagggtaatgaatcacttgctcttcctcgtcagtcgaggcgcggtggcataggagcctattcagttagaggttatcgccactctctaacgctcggatacgttggtcgatgttatcgatcatggtgcttaccccaatcgcgctgtattgctcatgcgcattttgaagatggcttcgcgctgtctgtagcgtgtctatatcccccaccgaagcggcgtggggtttaccttgctcatttcgaagcaagtgtaagccggcgaacttcaaccatttcgccgtcggcttctcattgatacgccacacgtgagtgaccttctcgaagacgagggagaaataaggttcaatggactgacctttgtctgccattcgctcagaccaggccaatacttcatcggcgcagaaggtcgcaaaatcacgcttgaatcgctcgggcgtatcgagcccgcgttcaatggcgatgtcacaccacttgatggcagtatcgagatcttcgatatcaaagagccaaatcaccatttgcgtaaacagcgggttatcgaactgcccatcaccggcgaggtaagcttcaatcgcgtcgcgatatttagggaccaacacttcgcgtttgtggttcaccttttcatctttacgattgaaggttttgagtaccttcaaatcgctgtcgagttcggcgagcaatagatgcaaactgtttgggttagcaacaaactgtatttcagctgtcgattgctgttgttgttttgccaaggcttcctggcgcaactttgctaatgggctggccatacattaccccttcgcagattcaacaacggtcacgtcttcaatcgcggcaaacttgtggtagttaccgacggcatacccttccatacgcaggtatgacgtttcaaagcgtttacggtcttcttcattgcgagacttacgccactgcgtccctttttgagtcaggatttgcaggttggttaagttcgttacccaaatcatattgggcgggaaaaatggtggggtatagacggttttgcctgccactgtcttcgccaaactttgcgcggctttgtgctcggtcggtacttccgccgattccaacaagcgatgctgctctgcagcgactaagttacgaccaatcagcacaaccaaatcaggatcgtcttgatgtacttcgtgaatcgtggtgttgattaaatcgttcacgagcgagtcgaggtttttatacgcgccgtctgtgttgcccgtagaatcaagcttcgcatcaggcaggacttgagccggcgccttttcttttgcaagctgcagccaacctttattaacgtcttcacccattgggttcgctttcgggtcggtgttttcacccgcaatcgaggttccgtgaaaaccaatacgcagcttatcgagcgcaaaattacgtgtgatagcgttattcatcagcttcatccactggcctttaccgcccgagttcgcccagatggtcatggtgatccaattgatgtgagcgcctgagtcggtctcggttaactcataggtgttaccactttggtcaagagcgcccatgaaacgaccgtctttgacacgccctgtcagtagacccgcgtcacccacatcaatcacttgacctttaatctggtcaaccgagatattggagatacgatttaaaaacgcgtccgattcgacaatcgcttggcgaagcctggtttccatcactggggtaatgttgaactgctttgaggcatccgctacaccgccggctttagccacggcttgacaatattcatctaaaaattgagtcgatacggcattaagcatttacaccacctccacaattgattcgccgccatggccttcttcactaggcttttgccctgggacttcttgcttaagctcggcgaactgggtttctaggctctttacttgctcggtgacgggcgcgagctgcttctccaattcactggaaaactgctctaaagagaacgtttgaacttcgccttcagggacggcttcgggttcattaggcgtttgcgtttgcaggttaaactcttgcttgagttcttcctttagctcacctttcatgatgccgaactgctctttcaggacagctttgagttgttcttcggtcacttcttcttcctctggttcaggttccggttgtggctctggctgttcgtcaccagaattgaaaaaggcattacacagagcaaaaaagcgatcggttttggaatagcactcatccaagttgatagcttccagttggctacaactgagctctgtggtgtggccttcttgtcgtgaaaactgaagtaatgacacaccagacgatgctggggaatcggtcacggctaaccccattaggtagcactttccttgccctttataatctggattgggttcaatggaggtaaacagcttctgcccaaacttattggcttcaagtaagtattgattaggttcaagcttggcaaacaagcgcattttgccgcccacttcttcggctttgacttcaagaactttgccccagttactgccgtaaccagcaaagcgtttatgctctggccaaatcaacgcggtgtattcactcagggcataattctcggcaatctgcgtgagccattcgcgggtgatcttccgaccatcaaccgttggcccttcggttgctacaattttccaatcgctgatttttgccatttgagtgtgtacctattgttcattggtcagtgcgtgttttgagttttcacaatacgcctttgaatcggccgtttcagccacttcaattccgaccaattcggatatgagcactatccgaaattatccgaactttgctatgcaatttagtgagttaactgggcttattattggctcatggcatatactcctgaaacacgacatgcagccagagccctgtatttaaaggcttggacacccaaagaaatcgcttccgagttaggtttgaacagcacacgaattatttatcactgggctgacaaacacggatggcgtgacatgctgcgcgaacaaacgattgatgagtcgatcgcgcgcagaattgaaaccctgcttgagttggaaaatcccaccaaaggccaactcgatatgcttgatagactcatcaagcaccatgtacagctaaaaaaatttcacgctcaagcccaatctacccaagagaaaaaagctcaaacagaaaaggaagcagcgactaacactcatcgaaataactcgcgttccaaaaagtctgacgacaaggcaaacaagaaaaaaagtaaaaagaagaaccacattgatgagctgaccaaagaaaactttgcaacctggcatgaatcactctttgaatatcagcacacgatgcgtaacaacctgcatcagcgtacacgtaacattctgaaatcacgtcagattggcgcgacctactatttcagtggtgaggcgctagaggacgcgattttgacgggtgacaaccagatatttttgtcagcgtctcgcgcccaggcagaagtatttagaagttacatcattgcgattgctcaagcgttcttaggcatcgaattgaccggcaacccgatcattctctctaatggcgcagagctgcgctttctttcgaccaactctaaaaccgcgcaaagttatcatggccacgtttatgtggatgagtacttttggatccctaaattcgatgagctcaacaaactcgcgtccgccatggcgacccacaagaactggcgaaaaacctacttctccaccccctcggctaaaacgcaccaggcttataccttttggacaggtgaccaatggcgccaaggtcgagatacccgcgccaatattgaatttccgacttttgacgactatcgagacggtggacgcctctgccctgacaagcagtggcgttacgtagttacaattgaggatgccgcagcaggaggctgtgagctttttgatattgatgagctgcgcgacgaatacagcaaagacgatttcgataatctgtttatgtgtatcttcgtcgatggcgccagctccgtctttaagttctcagcgcttgaaaaagccatggtggatattagccggtggcaagacttcaagcccaatgacaacgatcccttcgaccggcgtgaagtttggttaggttacgacccaagccgaacccgagacaacgcctgtttagttgtagtggccccgcctattgtcgccattgaaaagttccgagtcctggaaaagcactattggcgcgggttgaactttcagtaccaggctcagcaagtctcaaaagtgtttgagcgttacaacgtgagctatttgggcatcgataccacaggtattggcgcgggtgtgtatgacttactcagcaaaaagcacccgcgagaaaccgtggccattcaatacagcaacgagagtaaaaaccgattggtgatgaagatgattgatgtagtcgaagccaatcgcattcaatttgatgctgaacacaaagacattgccatggccttcatggccattaagcgggcgaccaccaacagcggtaacagcatgaccttcaaagctgagcgcagcgagttaaccggacacgccgacgcattctgggcgatttctcatgcctgcattaacgagccgctcgatcactcagaaaaacgcaaatcaacttggcagatgtaactctatgactgaacagaaaacagaaacaatcacaaatgacgaaagcttgatgtttagctttggtgagcctgagatcatgaatcgtgattttaccaactacgattacagcgagctcttttacaacgacgatggcgactattgggagccaccgcttgatagagcgggcttaaacaaactcacgcgagccaacgcctatcatggctctatcttaatggctcgacgtaatatgatcgctggccgttacattcaaggcggtatgcagaaacaacaaatgcagtcagctgtgcatgacttcttggagttcggtgatactgccctgcttaaactgcgtaactatttgggcaaagtcataggcctttggccaatccccaccatgtatttgcggaaacgtaagaacggtgacttcgcttttctagagcgggacaacaaacaaaagcgctacaagaaagaggacatcattttcatcaagcaatatgaccctgttcagcaggtttatggtgggcccgattacttagggtgtgttcaatctgcacttctaagccaggactcgaccacctttcgccgccggtactataagaacggtttgcacatgggctttatcttctacgccaccgacccaaacctaagtaaagaagatgaagatgacctgaaagaaaagatggcttcgagccgcggcgtaggtaactttcgttcgatgtttatcaatattccgaacggcaatgagagaggtatacagctcatacccgttggcgatattgccacaaaagatgaatatgaaaagattaagaacgtgactgcgcaagaggtgatcactggccaccgcttccccgttgagctcgctgccattattcctaatggtgggacccgtggtgatcctattaaattcgattacgtctactgcaaaaatgaagtcattccagcttgcgagatgttcatggatgccgttaacggtgatccggaagtgccggaaagtctgcatttaacctttaatctggacaacgttgccacgtaaacacagtgacattttttgcaattttgtttttctctccgattcacgctcagcccttacccagtaagggctgaagcgatacgaaactgatcatcaaaaaacacaaatgatcatttaaaaactgacctaaaatacagaaattcagatatttcagacacttaaaaagccacaacaggtcatcgcagatcgtcataatttcaatttattgcaactttttgcactcttcgcaatttcattagacgcaatataagccattctg

SEQ ID NO: 10 - DNA, 5085 bp, Vibrio campbellii
  source 1..5085: mol_type = genomic DNA
  misc_feature 1..5085: note = nucleotide sequence encoding bioABFCD
ttaagcgaagcaatcttgacgttgaagcgcagcatcaattgcgttaactagcttctcaatatcttccggcttgctaataaatggcggcatcatataaatcagtttgccaaatggacgaatccacacaccgtgctcaacaaacagtgcttggatggtttccatattcactgggcggtgtgtttctacaacgccaatggcacccaaccaacgggtgtttttaaccaagtcgtatttttccagttttggcagtagctcagaaaacagcgtttcaatttgctgggtttgtagctgccagttgccttgttctatcaattctaaacttgcggtggccacggcgcaggcgagcgggttacccataaaggttgggccgtgcataaaacacccagcatcaccgccacaaacggtatctgcgacgtgtttgctagctagggtcgctgagagggtcatgtaacctccagtgagcgctttaccgacacacagaatgtctggttgaatgtctgcatgttcgcaggcaaacaatttgccggtacgcccaaatccggttgcgatttcatctgcaatcaataacaagccgtatttgtcacaaagcctacgcacacctttgagaaattctggatggtagatacgcataccgcccgcaccttgaacgataggctctagaatcaccgcagcgagttcttgatgatgcgtttcaatcttgtgcgcaaagtcggcaagatcttccggcttccattcatcccaatagccacaagtaggtgactgggcaaagatatgctcaggcaaaaaacctttgtagagagagtgcatggaattatcaggatcggtgaccgacatagccgcaaaggtgtcgccgtgataaccgtgtcgcaaagtaagaaacttcggtcggcgttcgcccttcgcatgccaatattgcagtgccatcttcaagctgacttctacggcaacggagccagaatcagcgagaaagacatgttcaaggttacttggtgcaagggacaataacttcttacacaaactgattgctggctggtgggttatcccgccgaacattatatgcgacacctggtcgatttgctggtgagccgcttgatttaagtgcgggtggttgtagccgtggatcgttgaccaccaagaagacataccatcgacaagctctgtcccgtcttccaatttaatgtggacaccacttgccgatgccactgggtagcaggtcagaggtgtcaacgtcgaagtgtagggatgccagatgtgctggcggtcgaaggcgagatccatgtgctattccaatagtaattaagattaaaaaataaacagatgtaaacttttaggtttagtgttgtgttgacagtttatcgagactcagtagactgtcaacacaacaaaaaaagtgactaaaggaatacacgtggaagttcgtcataactggacgcatgctgaagtgcgcgatctcatggaaaaaccgttcatggatctgctattcgaagcacagcttgttcatcgtcaataccaacaaaccaactatgttcaagtaagcacgcttctttcgattaagacaggtgcttgtccggaagattgtaagtactgtcctcagagtgctcgttacaccacagacatagaaaaagagcgtctgatggaagtagaacgcgtactcgacgctgcgcaaaaagcgaaaaacgcgggttctactcgattctgtatgggcgcagcgtggaagaaccctaaagagcgtgatatgccgcacttgaccgacatgatcaaaggcgtgaaaggcatgggtttagaaacgtgtatgacgttaggcatgttgacgccagaccaagcaaagcaactggcaactgcaggtttggattactacaaccacaatctagatacttcacctgagttctacggcaacatcattaccacacgtacctatcaagaccgtttagataccttgtctcacgttcgtgatgccggcatgaagatttgttctggtggcattatcggtatgggtgagagtgccaatgaccgagcgggcttgttggttgaattagctaacttgccagtacaccctgaaagcgtgccaatcaacatgttggttaaggtgaaaggcacaccactggaagaggtggacgatgttgagccattcgattttattcgcttgattgcgattgcgcgcatcatgatgcctcaatcagcggttcgtctatcggctggccgtgaaaacatgaacgaacaaatgcaagcgctgtgttttatggcgggtgcaaactcagtcttctatggctgcaaacttctgacgactccaaacccttctgaagataaggacatgatgctgttcaataagctcggcattaacagccaagaagtgtctcaaaaacctgatgaaatagaagaaaacgagttgctggatcgcgtggtagagcgtgttactgctcgtccaacaaaagacgaccttttctacgatgccagcgtttaaatctcgtatcgagtccgcccttgccgaccgtaaagcgcaggggctgaatcgttcgattaatgtggtgttttccggcaaccaatcgattttggagtgcgaaggcagacgctacatcaacttctcaagtaacgactacttaggcttggccaatgaccaagcgcttgttcgtgcttggcaacaaggtttgagtgtatacggcagcggtagcggcgcctctccaatggtgacaggctttagtgcggcgcatagtaatttagaagctgcattgaccgaatggctgggttacgaacgggcaatcctgtttggctctggattcagtgccaatcaagcgttgttattaaccttgctagaaaagtcagacgtactcattcaagaccgtttgaaccatgcttctttgatggaggcggggatgttgtcgccagctaaaatgaagcgcttcaagcacaatgacatcgcgcatttaaattcgctgctgagtagcgaagacaatcatctggtcgtaactgaaggggtattcagtatggacggggattgtgcaccgcttgctgatatcgctgaagttacaagcagccacgatgcttggttcgccgtcgatgatgcacacggtattggcgttcttggtgaatccggtggtggttcttgcgagctggctaaagttaagcctgagttgttgatcgttacttttggcaaagctttcggtatgtccggcgcggctatcttatgtgaccacgcaacgggggactttttaactcagtttgctcgtcaccatgtgtattcaacggcgataccgccagcacaagcgtatgcgttgactcatgcagtctcgatgattcaagagcagtcttggcgacgcgaaaagcttacggaattaaatgaagcatatcgagatggtcttcaagatctcgatggttttgtcgaaacccaaacctcgattaagccctttatgattggtgagtctgaactggctttgcaggttgcaagtgcttgtcgacagaacggcatttgggtgactgctattcgtccacctacggtacctaaaggaacatctcgtcttcgaattacgctcacagccaatcacaccaacgaacaaattaaaacgctttcaatggcattgaagcaagcattaggggcgctgtaatggataatgcagaaaatatgacattggagagcttttaccaagacaaagaagcgatcgcctcttcgtttggaaaagcagcagaaacgtatgacaaacacgcggcgtttcagcgtgatgttggacatcgcttgcttgataaatttcctgaaaacttaacgggaaaacgcgtcttagatttggggtgtggtacaggctacttctctcaactgctccaacaacgtggcgcagaagtgatttgcggcgatatctctcaagcgatgttagataaagcgcagcagcgttgtggtacacagcaaatgcagtaccaaatagctgatgccgaaaacttgccatttgatgatgagagcttcgattacgttttttcaagtttggctttgcaatggtgtgtggatttaagctacccgttaagagagattcgtcgcgtcttaaaggccggtggggcggggtgtttctcaacattagccgatggctcactttatgagttgcgagaggcgtggtcaaaaattgatacatatcaacacgtgaataactttattacccacaatcaggtaaaaattgcgttagcgcaatcttggtgccacaaccatcatttagacttgacccccatcacagtttggtacgattcagcgttttctgtcatgcgtgatctcaaaggcattggtgcaaatcacgtaagcgggcgctcacatggtctgacgagtcgtcgcaccttgttgcaagttgagcgcgagtaccaagcatttagaaacgatcaaggtcttctacctgcaagttatcaggtttgttttggagtcatagttaaatgattgatgcattttttattgctggtacggatactgatgtaggaaaaacggtagcgtctaaagctattttacaagcactgggcgcgaaagggcttaacaccattggttacaagccagtggctgcgggaagcgacaagacggcggaaggttggcgtaactcggatgcattgcatctgcagaaagcggcaacactagatatcgcgtacgacgatgtaaacccatacgctttggaattacccacttctcctcacatcgccgctaagcacgaacatgtagaaattaaatacgaagtactgagcgaaaaattggctcaccataaagagcaatctgacattgttcttgtggaaggtgctggtggttggcgtgtgcctgtatctgatacagacagcctgtcgacatgggtacagcaagaacagcttccggtagttctggttgtaggtatcaagctggggtgtttgagccacgcactgttaacggcagagatcatcaaagcagatggtttgaatctcgttggctgggtagcgaaccgtgttaacccaggcacagagcactatgcagagatcatcgatatgctggaagatcgtttagatgctccaaaacttggtgagatcccatacattccaagtgcgaagcgtaaagatctaggcaaattcattaacgttgagccgttgctaaacgcgtaa

SEQ ID NO: 11 - DNA, 1257 bp, Vibrio campbellii
  source 1..1257: mol_type = genomic DNA
  misc_feature 1..1257: note = nucleotide sequence encoding hemA
atgtccttgcttgctattggaatcaatcacaatacagcgtctgttgacttgcgagagaaagtggcgttcggacccgataaattgggcccagcacttgagcaactcagagagcacgaagctgtcaatggcagtgtgattgtttctacctgtaaccgtaccgaagtgtactgcgacgtgaagcaaggagcgcgaaataagcttatcgactggctagcccagttccatcaagttaatcaagaagacttgatgccgagcctttatgtccatgaagagcaagcggcgattaagcacttgatgcgtgtttcttgcggccttgattcactggtacttggtgagcctcagattttgggtcaggtaaaacaagcgttttctgattcccgagatcatcaggcggtggatacgtcgattgataagttgttccaaaagactttttcagttgcaaaacgtgttcgaacagaaactgatattggtggtaacgcggtgtcggtggcttatgctgcctgtacattggcgaaacacatctttgaatcactgtctgactcaacggtactgttggttggtgcgggtgaaaccatcgaactggtggcgaaacacttagcgtctaacggctgtactaagatgattgttgctaaccgaacgcgagagcgtgctcttggtttgggtgagcaatttggcgcagaagtgatcagcttgaatgaaattcctgaccatttaccaagggcagacattgtcatcagctcaacggcgagtccattacctattattggtaagggcatggtagagactgcgttgaagaaacgtcgtcatcagccaatgcttttggtggatattgcggtcccacgtgatgtggaagcgcaggtgggtgaattgagtgatgcttacttgtactcggtggatgacttacagtcgatcatcgatagcaacatcgaacaacgtaaagtagaagcgattcaagcagaagcgatcgtcagcgaagaaagcgcggcatttatgacgtggctgcgttcactccaagccgtggacagtattcgtgattatcgcaaatcggctaacgaaattcgtgaagacttactaagtaagagcctacaatcattggcaaccggtgcagacccagaaaaagtgctgcgcgagcttagtaataaactcaccaacaaactgattcacgctccaactcgtgcgctgcaaagtgcagctgagcagggcgaaccagcaaaattaacggttatccgccagacccttggtttggatgatctgtaa

SEQ ID NO: 12 - DNA, 1044 bp, Vibrio campbellii
  source 1..1044: mol_type = genomic DNA
  misc_feature 1..1044: note = nucleotide sequence encoding recA
atggacgagaacaaacagaaagcgctcgccgctgcgctaggtcaaattgaaaagcaattcggtaaaggctctatcatgcgccttggtgataaccgcgcaatggacgtagaaaccatctcaacgggttctctttctcttgatatcgctttgggtgctggtggtctaccaatgggtcgtatcgtagaaatctacggtccagaatcttcaggtaaaacaacgctaactcttgagcttattgctgctgcgcaacgtgaaggcaaaacttgtgcgtttattgatgcagagcacgcgctagaccctgtatacgcaaagaaacttggcgttgatatcgatgcactattggtttctcagccagacacaggtgagcaagcgctagaaatctgtgacgcacttgcacgttctggtgctattgacgttatggttgtcgactctgttgctgcactaacaccaaaagcagaaatcgagggcgaaatgggcgatagccacatgggtcttcaagcacgtatgctttctcaagcgatgcgtaagctaacgggtaacttaaagcaatccaactgtatgtgtatcttcatcaaccaaatccgtatgaagattggtgtgatgttcggtaacccagaaacaacaactggtggtaacgcacttaagttctacgcttctgttcgtctagatatccgtcgtactggcgcaatcaaagaaggtgacgaagtagtaggtaacgaaacacgcatcaaagttgttaagaacaagattgcagcaccgtttaaagaagcgaacacgcaaatcatgtacggccaaggttttaaccgcgaaggcgaactgattgacctaggtgtgaagcacaagctagttgaaaaagcgggtgcatggtacagctaccaaggtgacaagatcggccaaggtaaagcaaacgcttgtaactacctacgcgaaaacccagaaatcggtaagactatcgatgctaaactgcgtgaaatgctactttcaccagcactacctgaagcaccagaatcgggtgaaaaatcagagcaagaagaagagttttaa

SEQ ID NO: 13 - DNA, 1278 bp, Vibrio campbellii
  source 1..1278: mol_type = genomic DNA
  misc_feature 1..1278: note = nucleotide sequence encoding bioA
atggatctcgccttcgaccgccagcacatctggcatccctacacttcgacgttgacacctctgacctgctacccagtggcatcggcaagtggtgtccacattaaattggaagacgggacagagcttgtcgatggtatgtcttcttggtggtcaacgatccacggctacaaccacccgcacttaaatcaagcggctcaccagcaaatcgaccaggtgtcgcatataatgttcggcgggataacccaccagccagcaatcagtttgtgtaagaagttattgtcccttgcaccaagtaaccttgaacatgtctttctcgctgattctggctccgttgccgtagaagtcagcttgaagatggcactgcaatattggcatgcgaagggcgaacgccgaccgaagtttcttactttgcgacacggttatcacggcgacacctttgcggctatgtcggtcaccgatcctgataattccatgcactctctctacaaaggttttttgcctgagcatatctttgcccagtcacctacttgtggctattgggatgaatggaagccggaagatcttgccgactttgcgcacaagattgaaacgcatcatcaagaactcgctgcggtgattctagagcctatcgttcaaggtgcgggcggtatgcgtatctaccatccagaatttctcaaaggtgtgcgtaggctttgtgacaaatacggcttgttattgattgcagatgaaatcgcaaccggatttgggcgtaccggcaaattgtttgcctgcgaacatgcagacattcaaccagacattctgtgtgtcggtaaagcgctcactggaggttacatgaccctctcagcgaccctagctagcaaacacgtcgcagataccgtttgtggcggtgatgctgggtgttttatgcacggcccaacctttatgggtaacccgctcgcctgcgccgtggccaccgcaagtttagaattgatagaacaaggcaactggcagctacaaacccagcaaattgaaacgctgttttctgagctactgccaaaactggaaaaatacgacttggttaaaaacacccgttggttgggtgccattggcgttgtagaaacacaccgcccagtgaatatggaaaccatccaagcactgtttgttgagcacggtgtgtggattcgtccatttggcaaactgatttatatgatgccgccatttattagcaagccggaagatattgagaagctagttaacgcaattgatgctgcgcttcaacgtcaagattgcttcgcttaa

SEQ ID NO: 14 - DNA, 1053 bp, Vibrio campbellii
  source 1..1053: mol_type = genomic DNA
  misc_feature 1..1053: note = nucleotide sequence encoding bioB
gtggaagttcgtcataactggacgcatgctgaagtgcgcgatctcatggaaaaaccgttcatggatctgctattcgaagcacagcttgttcatcgtcaataccaacaaaccaactatgttcaagtaagcacgcttctttcgattaagacaggtgcttgtccggaagattgtaagtactgtcctcagagtgctcgttacaccacagacatagaaaaagagcgtctgatggaagtagaacgcgtactcgacgctgcgcaaaaagcgaaaaacgcgggttctactcgattctgtatgggcgcagcgtggaagaaccctaaagagcgtgatatgccgcacttgaccgacatgatcaaaggcgtgaaaggcatgggtttagaaacgtgtatgacgttaggcatgttgacgccagaccaagcaaagcaactggcaactgcaggtttggattactacaaccacaatctagatacttcacctgagttctacggcaacatcattaccacacgtacctatcaagaccgtttagataccttgtctcacgttcgtgatgccggcatgaagatttgttctggtggcattatcggtatgggtgagagtgccaatgaccgagcgggcttgttggttgaattagctaacttgccagtacaccctgaaagcgtgccaatcaacatgttggttaaggtgaaaggcacaccactggaagaggtggacgatgttgagccattcgattttattcgcttgattgcgattgcgcgcatcatgatgcctcaatcagcggttcgtctatcggctggccgtgaaaacatgaacgaacaaatgcaagcgctgtgttttatggcgggtgcaaactcagtcttctatggctgcaaacttctgacgactccaaacccttctgaagataaggacatgatgctgttcaataagctcggcattaacagccaagaagtgtctcaaaaacctgatgaaatagaagaaaacgagttgctggatcgcgtggtagagcgtgttactgctcgtccaacaaaagacgaccttttctacgatgccagcgtttaa

SEQ ID NO: 15 - DNA, 1152 bp, Vibrio campbellii
  source 1..1152: mol_type = genomic DNA
  misc_feature 1..1152: note = nucleotide sequence encoding bioF
atgccagcgtttaaatctcgtatcgagtccgcccttgccgaccgtaaagcgcaggggctgaatcgttcgattaatgtggtgttttccggcaaccaatcgattttggagtgcgaaggcagacgctacatcaacttctcaagtaacgactacttaggcttggccaatgaccaagcgcttgttcgtgcttggcaacaaggtttgagtgtatacggcagcggtagcggcgcctctccaatggtgacaggctttagtgcggcgcatagtaatttagaagctgcattgaccgaatggctgggttacgaacgggcaatcctgtttggctctggattcagtgccaatcaagcgttgttattaaccttgctagaaaagtcagacgtactcattcaagaccgtttgaaccatgcttctttgatggaggcggggatgttgtcgccagctaaaatgaagcgcttcaagcacaatgacatcgcgcatttaaattcgctgctgagtagcgaagacaatcatctggtcgtaactgaaggggtattcagtatggacggggattgtgcaccgcttgctgatatcgctgaagttacaagcagccacgatgcttggttcgccgtcgatgatgcacacggtattggcgttcttggtgaatccggtggtggttcttgcgagctggctaaagttaagcctgagttgttgatcgttacttttggcaaagctttcggtatgtccggcgcggctatcttatgtgaccacgcaacgggggactttttaactcagtttgctcgtcaccatgtgtattcaacggcgataccgccagcacaagcgtatgcgttgactcatgcagtctcgatgattcaagagcagtcttggcgacgcgaaaagcttacggaattaaatgaagcatatcgagatggtcttcaagatctcgatggttttgtcgaaacccaaacctcgattaagccctttatgattggtgagtctgaactggctttgcaggttgcaagtgcttgtcgacagaacggcatttgggtgactgctattcgtccacctacggtacctaaaggaacatctcgtcttcgaattacgctcacagccaatcacaccaacgaacaaattaaaacgctttcaatggcattgaagcaagcattaggggcgctgtaa

SEQ ID NO: 16 - DNA, 807 bp, Vibrio campbellii
  source 1..807: mol_type = genomic DNA
  misc_feature 1..807: note = nucleotide sequence encoding bioC
atggataatgcagaaaatatgacattggagagcttttaccaagacaaagaagcgatcgcctcttcgtttggaaaagcagcagaaacgtatgacaaacacgcggcgtttcagcgtgatgttggacatcgcttgcttgataaatttcctgaaaacttaacgggaaaacgcgtcttagatttggggtgtggtacaggctacttctctcaactgctccaacaacgtggcgcagaagtgatttgcggcgatatctctcaagcgatgttagataaagcgcagcagcgttgtggtacacagcaaatgcagtaccaaatagctgatgccgaaaacttgccatttgatgatgagagcttcgattacgttttttcaagtttggctttgcaatggtgtgtggatttaagctacccgttaagagagattcgtcgcgtcttaaaggccggtggggcggggtgtttctcaacattagccgatggctcactttatgagttgcgagaggcgtggtcaaaaattgatacatatcaacacgtgaataactttattacccacaatcaggtaaaaattgcgttagcgcaatcttggtgccacaaccatcatttagacttgacccccatcacagtttggtacgattcagcgttttctgtcatgcgtgatctcaaaggcattggtgcaaatcacgtaagcgggcgctcacatggtctgacgagtcgtcgcaccttgttgcaagttgagcgcgagtaccaagcatttagaaacgatcaaggtcttctacctgcaagttatcaggtttgttttggagtcatagttaaatga

SEQ ID NO: 17 - DNA, 684 bp, Vibrio campbellii
  source 1..684: mol_type = genomic DNA
  misc_feature 1..684: note = nucleotide sequence encoding bioD
atgattgatgcattttttattgctggtacggatactgatgtaggaaaaacggtagcgtctaaagctattttacaagcactgggcgcgaaagggcttaacaccattggttacaagccagtggctgcgggaagcgacaagacggcggaaggttggcgtaactcggatgcattgcatctgcagaaagcggcaacactagatatcgcgtacgacgatgtaaacccatacgctttggaattacccacttctcctcacatcgccgctaagcacgaacatgtagaaattaaatacgaagtactgagcgaaaaattggctcaccataaagagcaatctgacattgttcttgtggaaggtgctggtggttggcgtgtgcctgtatctgatacagacagcctgtcgacatgggtacagcaagaacagcttccggtagttctggttgtaggtatcaagctggggtgtttgagccacgcactgttaacggcagagatcatcaaagcagatggtttgaatctcgttggctgggtagcgaaccgtgttaacccaggcacagagcactatgcagagatcatcgatatgctggaagatcgtttagatgctccaaaacttggtgagatcccatacattccaagtgcgaagcgtaaagatctaggcaaattcattaacgttgagccgttgctaaacgcgtaa

SEQ ID NO: 18 - DNA, 1098 bp, Vibrio campbellii
  source 1..1098: mol_type = genomic DNA
  misc_feature 1..1098: note = nucleotide sequence encoding luxP
atgaagaaagcgttactattttccctgatttctatggtcggtttttctccagcgtctcaagcaacacaagttttgaatgggtactggggttatcaagagtttttggacgagtttcccgagcaacgaaatctgaccaatgctttatcagaagcagtacgagcacagccggtcccactgtctaaaccgacacaacgcccgattaaaatatcagtggtttacccaggacagcaagtttcagattactgggtaagaaatattgcatcattcgaaaaacgtttgtataagttgaatattaactaccaactgaaccaagtgtttactcgtccaaatgctgatatcaagcaacaaagcttgtcattaatggaagcgctcaagagcaaatcggattacttgattttcacgcttgatacgacaagacaccgtaaatttgttgagcacgttttggactcaacgaacaccaaattgatcttgcaaaatatcactacaccagtccgtgagtgggacaaacatcaaccgtttttatatgtcggatttgaccacgcagaaggcagtcgtgaattagcaacagaattcggaaagttcttcccaaaacacacatattacagtgtgctctacttttctgaaggttatattagcgatgtgagaggtgatacttttattcaccaagtaaaccgtgataataactttgagctacaatcagcgtattacacgaaggcaaccaagcaatccggctatgatgctgcgaaagcgagtttagcaaaacatccagatgttgattttatctatgcatgttcgaccgacgtagcattaggtgcagtagacgcactggctgagttgggacgtgaagatattatgatcaatggctggggtggaggctctgctgagttagacgctatccagaagggtgatttagacatcaccgtcatgcgtatgaatgatgacactggcatagccatggcagaagcgattaagtgggacttggaagataaaccagttccgaccgtatactcaggtgactttgaaatcgtaacaaaggcagattcaccggagagaatcgaagcgctgaaaaagcgcgcatttagatattcagataattga

SEQ ID NO: 19 - DNA, 2580 bp, Vibrio campbellii
  source 1..2580: mol_type = genomic DNA
  misc_feature 1..2580: note = nucleotide sequence encoding luxQ
atgacaacaacgcgatcaaacattaaaaagcgtcgctcgctggcgacgctcataacaaagatcatcattttagttcttgccccaattattctggggattttcattcagagctattacttctccaagcaaatcatttggcaagaagtagaccgaaccaaacagcaaacctctgcactgatccacaatatatttgatagccactttgcggcgatccagatacatcatgacagtaattccaagagcgaagtcattcgtgatttctacactgatcgcgacacggatgtgctcaactttttcttcctcagtatcgaccaaagcgatccgtcgcacacaccagaattccgttttctaacggaccacaaaggcatcatttgggacgatggaaatgcgcatttctatggtgtgaacgaccttatccttgatagccttgccaatcgggtcagtttcagtaacaactggtattacattaatgtcatgacctccattggttctagacacatgctcgtgcgccgtgtgccgatcctagacccttcaacaggagaggtgcttggtttctcatttaatgccgtcgtcttagacaacaacttcgctttgatggaaaagctcaagagtgaaagtaacgtcgacaatgtggtgctggttgctaatagcgttcctttagcaaactctttgattggtgatgagccatataacgttgctgatgtattgcagcgtaaaagttcagacaaaagactcgataagctgttggtaatagaaacgccaatcgtcgtaaatgcagtgactaccgagctttgcttgttgacggtacaagacaatcagagtgtggtgacattacaaatccaacatattctagccatgcttgcatcgatcatcggtatgatcatgattgccttaatgagtagggaatggattgagagtaaagtttcggcgcagttagaatctttgatgtcttacacccgctctgctcgtgaggaaaaagggtttgaacgatttggcggttcggatattgaagagtttgatcacatcggttcaacccttgaaagtacattcgaagagcttgaagcgcagaagaagtcgttccgagatctgtttaattttgccttatcacccatcatggtttggtctgaagagagtgtcctgattcagatgaaccctgccgcgcgcaaagaattagtgatcgaagacgatcatgaaatcatgcatccggtcttccaaggctttaaagagaaattgaccccacacctcaaaatggcggctcaaggtgcgacgttgactggggtgaacgtgcctattggtaataagatctaccgatggaacttgtcgccaattcgtgttgatggcgatatcagtggcattattgtgcaaggccaagacattacaacacttatcgaagccgagaagcagagtaacattgcgcgtagagaagcagaaaaatcggcgcaagcacgtgcagacttccttgctaaaatgagccatgaaattcgtacgccaatcaacggcattttaggtgtcgcccaattattgaaagattctgtcgatacacaagagcagaagaatcaaatcgacgtcctgtgccacagtggcgagcacttgcttgcagtactgaacgatattctcgatttctcaaagatagagcagggcaagttcaatattcagaaacacccgttctccttcaccgataccatgcgtacattggaaaatatttatcgtccgatttgcacaaataagggggtggagttggtcatcgagaatgagcttgacccgaatgttgaaatcttcaccgatcaagtccgcttgaatcagattctatttaacttagtgagtaatgccgttaagttcacgccgattggctcgattcgactgcacgcagaacttgaacaattctatggtgcggagaacagcgtgttagttgtggaactgactgatactggcatcggcattgaaagcgataagctcgaccaaatgttcgaaccttttgtgcaagaagagtcgacaaccacacgcgaatatggcggtagcggcctaggtttgaccatcgttaagaacctagtcgatatgttagaaggtgatgttcaggtccgcagtagcaaggggggggggacaacatttgttataacacttccagtaaaagatcgtgagcgtgtcttaaggcctctggaggtcagtcaacgtatcaagccggaagccttgtttgatgaaagtttaaaagtgctactggtggaagataaccataccaatgcgtttatccttcaggctttctgtaagaagtataaaatgcaggtggattgggcgaaagatgggctggacgcgatggagctcctttctgataccacctacgatctgatcctcatggataaccaattaccccaccttggtggtattgagaccacgcacgagattcgccagaacttgaggcttggaacgccaatttacgcgtgtacagcagacaccgcgaaagaaaccagtgatgcgtttatggcggcaggtgcaaactatgtcatgctgaagccaattaaagagaatgcgttacatgaggcgtttgtcgatttcaaacaacgtttcttggtagaaagaacctaa

SEQ ID NO: 20 - DNA, 1068 bp, Vibrio campbellii
  source 1..1068: mol_type = genomic DNA
  misc_feature 1..1068: note = nucleotide sequence encoding luxA
atgaaatttggaaacttccttctcacttatcagccacctgagctatctcagaccgaagtgatgaagcgattggttaatctgggcaaagcgtctgaaggttgtgggttcgacactgtttggttactagagcaccacttcactgaatttgggttgttagggaatccttatgttgctgccgcacatctattaggtgcgacagaaaagctcaatgttggcaccgcagctattgtattgccgactgcccatccggttcgacaagcagaagacgtaaacctactggatcaaatgtcaaaaggacgattccgttttggtatttgtcgcggtttgtacgacaaagattttcgtgtctttggtacagacatggataacagccgagccttaatggactgttggtatgacttgatgaaagaaggcttcaatgaaggctatatcgcggcggataacgaacatattcagttcccgaaaatccaactgaatccatcggcttacacacaaggtggcgctcctgtttatgtcgtcgcggagtcagcgtcaacgacagaatgggctgcagagcgtggcctaccaatgattctaagctggatcatcaacactcacgagaagaaagcgcagcttgatctttacaatgaagttgcgactgaacatggctacgatgtgactaagattgaccactgtttgtcttacatcacttccgtcgatcatgactcaaataaagccaaagatatttgccgcaacttcttgggtcattggtacgactcatacgtgaatgccacaaagatttttgacgactctgaccaaacaaaaggctacgacttcaataaaggtcaatggcgtgattttgtcttgaaaggccacaaagacaccaatcgccgaattgattacagctacgaaatcaacccagtaggaacgcctgaagagtgtatcgcgattatccagcaagatatcgatgcgacgggtattaacaatatttgttgtggttttgaagcaaacggttctgaagaagaaattatcgcatctatgaagctattccagtctgatgtgatgccatatctcaaagaaaaacagtaa

SEQ ID NO: 21 - DNA, 19 bp, Vibrio campbellii
  source 1..19: mol_type = genomic DNA
  misc_feature 1..19: note = 5' sequence; glmS-
atgtgtggaatcgtaggtg

SEQ ID NO: 22 - DNA, 19 bp, Vibrio campbellii
  source 1..19: mol_type = genomic DNA
  misc_feature 1..19: note = 3' sequence; glmS-
agcggtaaccgtagagtaa

SEQ ID NO: 23 - DNA, 15 bp, Vibrio campbellii
  source 1..15: mol_type = genomic DNA
  misc_feature 1..15: note = 5' sequence; thyA-
gtgaaacagtattta

SEQ ID NO: 24 - DNA, 15 bp, Vibrio campbellii
  source 1..15: mol_type = genomic DNA
  misc_feature 1..15: note = 3' sequence; thyA-
cctttctcagtttaa

SEQ ID NO: 25 - DNA, 19 bp, Vibrio campbellii
  source 1..19: mol_type = genomic DNA
  misc_feature 1..19: note = 5' sequence; cqsS-
gtgctaggctcagtagata

SEQ ID NO: 26 - DNA, 139 bp, Vibrio campbellii
  source 1..139: mol_type = genomic DNA
  misc_feature 1..139: note = 3' sequence; cqsS-
ttcggtacctattattgcgcatactggtgatagctcaccgataacactggacaagattggttcatctggcatgtcggatttcatcgttaaacctgcggataagaacaggttgttcgacaagattgcgaactggatttag

SEQ ID NO: 27 - DNA, 12 bp, Vibrio campbellii
  source 1..12: mol_type = genomic DNA
  misc_feature 1..12: note = 5' sequence; luxPQ-
atgaagaaagcg

SEQ ID NO: 28 - DNA, 22 bp, Vibrio campbellii
  source 1..22: mol_type = genomic DNA
  misc_feature 1..22: note = 3' sequence; luxPQ-
cggtttaatggcagtgataagt

SEQ ID NO: 29 - DNA, 50 bp, Vibrio campbellii
  source 1..50: mol_type = genomic DNA
  misc_feature 1..50: note = 5' sequence; delta Hap9427
atgcacatcaaacaccggagtcagccttgccatatttggcagcagaccga

SEQ ID NO: 30 - DNA, 50 bp, Vibrio campbellii
  source 1..50: mol_type = genomic DNA
  misc_feature 1..50: note = 3' sequence; delta Hap9427
tagggtatgtgctccccttaccaactaattcttctttagttcattaccgc

SEQ ID NO: 31 - DNA, 50 bp, Vibrio campbellii
  source 1..50: mol_type = genomic DNA
  misc_feature 1..50: note = 5' sequence; delta Kap10060
caacaaggtagccaaggcaaccagagccgatagccaagttgggtccatcg

SEQ ID NO: 32 - DNA, 50 bp, Vibrio campbellii
  source 1..50: mol_type = genomic DNA
  misc_feature 1..50: note = 3' sequence; delta Kap10060
agcaccattaaccttcagcaatcccctttattactaaagggctaacggct

SEQ ID NO: 33 - DNA, 50 bp, Vibrio campbellii
  source 1..50: mol_type = genomic DNA
  misc_feature 1..50: note = 5' sequence; delta bioABFCD
gtttttattttttaagtgttcttcaaatttaatgagcgcattaagcgaag

SEQ ID NO: 34 - DNA, 50 bp, Vibrio campbellii
  source 1..50: mol_type = genomic DNA
  misc_feature 1..50: note = 3' sequence; delta bioABFCD
aaacgcgtaaaggcttcgcgattcgataaatacaaaaagggctgcgcaat

SEQ ID NO: 35 - DNA, 21 bp, Vibrio campbellii
  source 1..21: mol_type = genomic DNA
  misc_feature 1..21: note = 5' sequence; hemA-
atgtccttgcttgctattgga

SEQ ID NO: 36 - DNA, 12 bp, Vibrio campbellii
  source 1..12: mol_type = genomic DNA
  misc_feature 1..12: note = 3' sequence; hemA-
gatgatctgtaa

SEQ ID NO: 37 - DNA, 15 bp, Vibrio campbellii
  source 1..15: mol_type = genomic DNA
  misc_feature 1..15: note = 5' sequence; luxM-
atgaaattaatgttg

SEQ ID NO: 38 - DNA, 58 bp, Vibrio campbellii
  source 1..58: mol_type = genomic DNA
  misc_feature 1..58: note = 3' sequence; luxM-
acgtgctgttcgagcgagcagaaaacgcggttcgttagaaagggatgagcatgtttga

SEQ ID NO: 39 - DNA, 19 bp, Vibrio campbellii
  source 1..19: mol_type = genomic DNA
  misc_feature 1..19: note = 5' sequence; luxB-
atgaaatttggattattct

SEQ ID NO: 40 - DNA, 19 bp, Vibrio campbellii
  source 1..19: mol_type = genomic DNA
  misc_feature 1..19: note = 3' sequence; luxB-
cgtcaaataccactcgtaa

SEQ ID NO: 41 - DNA, 53 bp, Vibrio campbellii
  source 1..53: mol_type = genomic DNA
  misc_feature 1..53: note = luxN H471A
cgcgcactagctaactctattgctcacgaaatgcgtaaccctcttgctcaagt

SEQ ID NO: 42 - DNA, 15 bp, Vibrio campbellii
  source 1..15: mol_type = genomic DNA
  misc_feature 1..15: note = 5' sequence; recA-
aaagtgatggacgag

SEQ ID NO: 43 - DNA, 18 bp, Vibrio campbellii
  source 1..18: mol_type = genomic DNA
  misc_feature 1..18: note = 3' sequence; recA-
gaagagttttaatcttct

(19) United States
(12) Patent Application Publication
(10) Pub. No.: US 2026/0035729 A1
(43) Pub. Date: Feb. 5, 2026

(54) CREATING A VIBRIO-BASED EDUCATIONAL TOOLSET FOR PROTEIN SIGNALING

(71) Applicants: BIO-RAD LABORATORIES, INC., HERCULES, CA (US); THE TRUSTEES OF INDIANA UNIVERSITY, BLOOMINGTON, IN (US)

(72) Inventors: GEORGE CHENAUX, HERCULES, CA (US); JEAN SPAGNOLO-SMITH, HERCULES, CA (US); ASHLEIGH MILLER, HERCULES, CA (US); DELQUIN GONG, HERCULES, CA (US); JULIA C. van KESSEL, BLOOMINGTON, IN (US)

(21) Appl. No.: 19/285,104

(22) Filed: Jul. 30, 2025

Related U.S. Application Data
(60) Provisional application No. 63/677,175, filed on Jul. 30, 2024.

(51) Int. Cl. C12Q 1/02; C12N 1/20; C12N 1/36; C12Q 1/24

(52) U.S. Cl. CPC C12Q 1/025; C12N 1/20; C12N 1/36; C12Q 1/24

(57) ABSTRACT

Described herein are recombinant, attenuated bacteria with an altered quorum sensing pathway. The recombinant bacteria are used for visualizing quorum sensing or immunizing marine organisms against infections by Vibrio spp.

### CROSS-REFERENCE TO RELATED APPLICATION

This application claims the benefit of U.S. Provisional Application Ser. No. 63/677,175, filed Jul. 30, 2024, the disclosure of which is hereby incorporated by reference in its entirety, including all figures, tables and amino acid or nucleic acid sequences.

### STATEMENT REGARDING SEQUENCE LISTING

The Sequence Listing for this application is labeled “Seq-List.xml” which was created on Jul. 30, 2025 and is 105,536 bytes. The entire content of the sequence listing is incorporated herein by reference in its entirety.

### BACKGROUND OF THE INVENTION

Hands-on, wet-lab activities can be useful tools to allow individuals to directly investigate various biological processes, including cell signaling pathways. Currently, no wet-lab activities exist in which students can study a protein signaling pathway that exhibits a clear, visual result (for example, a result that is visible to the naked eye) in a classroom setting. Bacteria can be used to study cell signaling pathways, but most bacteria require proper handling and disposal to ensure the safety of individuals. Therefore, creating bacteria that are safe, even if individuals do not properly handle and dispose of the bacteria, is essential. Finally, protein signaling pathways are often complex, requiring many steps, including the binding of ligands in a cascading manner, which can be affected by a variety of factors. Therefore, ensuring a simple, reliable, and visually appealing event that demonstrates the protein signaling pathway in a safe and reliable manner is urgently needed.

### BRIEF SUMMARY OF THE INVENTION

The subject invention provides recombinant bacteria that possess genetic mutations that attenuate the bacteria and/or introduce an auxotrophy and alter the quorum sensing pathway. In various embodiments, the bacteria is a Vibrio sp., including, for example, Aliivibrio fischeri (A. fischeri), Vibrio campbellii (V. campbellii), Vibrio harveyi (V. harveyi), Vibrio alginolyticus (V. alginolyticus), Vibrio anguillarum (V. anguillarum), and Vibrio fluvialis (V. fluvialis). In other embodiments, the bacteria is Aliivibrio fischeri (formerly Vibrio fischeri). In certain other embodiments, the bacteria is V. harveyi or V. campbellii. In various embodiments, one or more genes in the quorum sensing pathway and one or more genes are mutated such that the recombinant bacteria is altered such that it is an auxotrophic mutant dependent on one or more compounds for growth. In some embodiments, the recombinant bacteria may depend on at least two compounds to regulate the growth and virulence of the bacteria and, by controlling the expression of multiple genes, allow for biological containment. The dependence on multiple compounds for growth and/or virulence enhances the safety of the recombinant bacteria, given the improbability that the organisms will encounter these compounds in a naturally occurring environment.

In certain embodiments, the recombinant bacteria is an auxotrophic mutant for at least 1, 2, 3, 4, 5, 6, 7, 8, 9, 10 or more essential compounds that are prototrophically produced in a corresponding wild-type (unmutated) bacteria. In certain embodiments, the essential compound is glucosamine, biotin, 5-aminolevulinic acid (5-ALA), thymidine, or a combination thereof. In certain embodiments, the glmS locus can be inactivated in the bacterium, whereby glucosamine synthase is not synthesized and fructose-6-phosphate is not converted to glucosamine-6-phosphate. In certain embodiments, the thyA locus can be inactivated, whereby thymidylate synthase is not synthesized and deoxyuridine monophosphate (dUMP) is not converted into deoxythymidine monophosphate (dTMP). In yet other embodiments, one or more of the genes associated with biotin synthesis can be inactivated or deleted (e.g., bioA, bioB, bioF, bioC, and/or bioD can be inactivated or deleted within the bacteria). In some embodiments, the entire biotin locus (bioABFCD)) can be inactivated or deleted from the genome of the bacterial cell and, in other embodiments, one or both of bioA and/or bio can be inactivated. Yet other embodiments provide for the inactivation or deletion of the hemA gene in a bacterial cell, which results in the bacterial cell being unable to express a glutamyl tRNA reductase, an essential enzyme for synthesis of aminolevulinic acid, a precursor for heme biosynthesis. The inactivation of the hemA gene requires supplementation of 5-aminolevulinic acid to facilitate bacterial growth and survival. In yet other embodiments, the recA gene is inactivated or deleted such that recombinational DNA repair is impaired within the bacterial cell.

In various embodiments, a single gene or more than one gene can be inactivated to create an auxotrophic mutant. Thus, one or more of glmS, thyA, bioABFCD, and/or hemA can be inactivated to create an auxotrophic mutant.

The bacterial cells may further comprise the inactivation or deletion of the recA gene. Table 1 illustrates various gene combinations that can be inactivated in order to create an auxotrophic mutant as disclosed herein that further comprises inactivation or deletion of the recA gene.

Additionally, one or more of bioA, bioB, bioF, bioC, and/or bioD can be inactivated or deleted or the entire bioABFCD) operon can be inactivated in the creation of the auxotrophic mutant. For the purposes of Table 1, the bioABF (D) designation is intended to include each individual gene of the bioABFCD operon (bioA, bioB, bioF, bioC, and/or bioD) as well as the entire operon for the purposes of inactivation to introduce an auxotrophic mutation within the bacterium. In certain embodiments, the bacterial cells provided herein are glmS−, thyA−, ΔbioABFCD, and recA−. In other words, the bacterial cells contain mutations that inactivate or delete glmS, thyA, bioABFCD) to create the auxotrophic mutant and recA.

In certain embodiments, the recombinant bacteria further comprise modifications that reduce or inhibit the ability for a bacteriophage that is integrated into the bacterial chromosome to form. For example, modifications of DNA encoding phage capsid (head) proteins or phage tail proteins can be included in the disclosed recombinant bacterial cells (e.g., such as deletions within the DNA encoding phage capsid (head) proteins or tail proteins or insertions within the DNA encoding phage capsid proteins or tail proteins, for example, the insertion of one or more stop codon within the DNA encoding the phage capsid (head) proteins or tail proteins). In certain embodiments, the bacterium comprises a mutation or insertion in the Φ-HAP-1-like bacteriophage region as disclosed herein (e.g., SEQ ID NO: 8) or a deletion of the Φ-HAP-1-like bacteriophage region or any portion thereof. In certain embodiments, the bacterium comprises a mutation or insertion in the Kappa-like phage region (e.g., SEQ ID NO: 9) or a deletion of Kappa-like phage region or any portion thereof as disclosed herein. In preferred embodiments, the bacterium comprises a mutation in the Φ-HAP-1-like bacteriophage region and the Kappa-like phage region. In other embodiments, any chromosomal sequence resembling a phage capsid and/or tail region can be modified such that the bacteriophage cannot form.

In certain embodiments, the recombinant bacteria also comprise modifications that alter the quorum sensing pathway. In certain embodiments, the recombinant bacteria cannot sense cholera autoinducer-1 (CAI-1), autoinducer-2 (AI-2), harveyi autoinducer-1 (HAI-1), or any combination thereof. In certain embodiments, the bacterium possesses a deletion, mutation, or insertion in the cqsS gene, the luxPQ genes, the luxN gene, the luxM gene, the luxA gene, the luxB gene, or any combination thereof that decreases or eliminates expression of the gene or any combination of genes or any orthologs thereof. In various embodiments, the cqsS gene, the luxPQ genes, the luxN gene, the luxM gene, the luxA gene, the luxB gene can contain mutations that alter the function of the enzyme or protein expressed by the gene (e.g., the LuxN-H471A mutation described herein). In certain embodiments, the bacterium comprises a deletion in the cqsS gene, the luxPQ gene, the luxN gene, the luxM gene, the luxA gene, the luxB gene, any orthologs thereof, or any combination thereof that decreases or eliminates expression of the gene or any combination of genes. In preferred embodiments, the bacterium comprises deletions of the cqsS gene and the luxPQ genes or any orthologs thereof.

In certain embodiments, the recombinant bacterial strains can be provided in a kit in combination with instructions for using the kit components. In further embodiments, the kit comprises prepared media or the components to synthesize the media for growing the bacteria.

The instant disclosure further provides recombinant bacteria with desirable safety features that can be safely used to effectively deliver antigenic compounds to a subject (e.g., fish or shellfish, such as shrimp) in order to mount potent immunogenic responses against pathogens, such as Vibrio harveyi, V. anguillarium, V. vulnificus, or other pathogenic Vibrio spp.

### BRIEF DESCRIPTION OF THE DRAWINGS

The patent or application file contains at least one drawing executed in color. Copies of this patent or patent application publication, with color drawing(s), will be provided by the Office upon request and payment of the necessary fee. One or more of the figures was created with BioRender.com.

FIG. 1A shows the growth of wild-type (unmutated) Vibrio campbellii and recombinant V. campbellii strains that have genes glmS and thyA inactivated. The growth is shown in media without glucosamine (Gl) or thymidine (Th), media with glucosamine and without thymidine, media with thymidine and without glucosamine, or media with glucosamine and thymidine.

FIG. 1B shows the growth of two recombinant V. campbellii strains. bioABFCD)+ strain retains functional biotin synthesis operon loci: bioA, bioB, biol, bioC, and bioD). The ΔbioABFCD) strain does not retain any of those listed functional biotin synthesis operon loci. The growth is shown in media with or without biotin supplementation.

FIG. 1C shows the growth of two recombinant V. campbellii strains: hemA+ strain with a functional hemA locus and hemA-strain, which lacks a functional hemA locus. The growth is shown in media with or without aminolevulinic acid (ALA) supplementation. The bacteria were imaged in the light (bright field) and in the dark (luminescence).

FIG. 2 shows a simplified quorum sensing pathway in a recombinant V. campbellii strain, which has genes cqsS and luxPQ inactivated, in both low density ligand conditions and high density ligand conditions. While not a true wild-type strain, this is the recombinant wild-type proxy strain used in activities that retains the ability to be used to interrogate key steps of the V. campbellii quorum sensing lux-operon pathway. In this strain, LuxM synthase produces ligand HAI-1 (extracellular circles) that binds to the LuxN receptor, which decreases its phosphorylation transfer to the second messenger LuxO and allows for phosphatase to deplete the pool of phosphorylated LuxO. Once LuxO is dephosphorylated, there is a decrease in the repression of the transcription factor LuxR, and the subsequent increase in expression of LuxR increases the expression of the lux operon genes including the luciferase enzymes LuxA and LuxB, whose expression allows for luminescence.

FIG. 3 shows co-plating of recombinant wild-type proxy V. campbellii (WT) and recombinant quorum sensing disrupted V. campbellii daughter strains luxM-, luxB-, and luxN-H471A (H471A). The bacteria were imaged in the light (bright field) and in the dark (luminescent). WT and H471A cultures demonstrate luminescence throughout the entirety of the cultures, and H471A is slightly brighter than WT. luxM-cultures only show luminescence at the border with neighboring cultures. luxB-cultures have no luminescence.

FIG. 4 shows plating of recombinant wild-type proxy V. campbellii (WT) and recombinant quorum sensing disrupted V. campbellii daughter strains luxM-, luxB-, and luxN-H471A (H471A) that were treated with synthetic ligand harveyi autoinducer-1 (HAI-1), LuxN inhibitor C450-0730 (C450), LuxR inhibitor Qstatin (Qstat), and solvent-only control (Ctrol). The bacteria were imaged in the light (bright field) and in the dark (luminescent). WT strain cultures luminescence levels are as follows: Ctrol, strong luminescence in entire quadrant; HAI-1, strong luminescence in entire quadrant; Qstat and C450, minimal luminescence in entire quadrant. luxM-strain cultures luminescence levels are as follows: Ctrol, luminescence only visible near border of HAI-1 treated quadrant; HAI-1, strong luminescence in entire quadrant; Qstat and C450, no luminescence in entire quadrant. H471A strain cultures luminescence levels are as follows: Ctrol, HAI-1, & C450, strong luminescence in entire quadrant; Qstat, reduced luminescence in entire quadrant. luxB-strain cultures luminescence levels are as follows: Ctrol, HAI-1, C450, & Qstat, no luminescence in all quadrants.

FIG. 5 shows the plating of a recombinant V. campbellii strain, which has genes cqsS, luxM, and luxPQ inactivated, on agar plate media treated with 10 nmol and 100 nmol (low and high, respectively) of synthetic ligand, (HAI-1). Plate cultures were exposed to the following: with zero or 50 nmol of luxN inhibitor C450-0730 (C450, − or +, respectively) or with zero or 50 nmol of LuxR inhibitor PTSP (− or +, respectively). The bacteria were imaged in the light (bright field) and in the dark (luminescent). In Low HAI-1 treated cultures, luminescence is visible near the region of the HAI-1 treated quadrant unless cells are also treated with C450 or PTSP. In High HAI-1 treated cultures, luminescence levels are higher without additional treatment, is visible in C450 treated cultures, and is near-absent in PTSP treated cultures.

FIG. 6 shows crystal violet stains of culture tubes indicating the accumulation of biofilms. Three tubes shown housed recombinant V. campbellii strains lacking cqsS and luxPQ loci. Cultures were untreated (none), treated with synthetic ligand harveyi autoinducer-1 (HAI-1), or treated with LuxN inhibitor C450-0730 (C450). A tube treated with ligand HAI-1 added to the cells shows a decrease in biofilm accumulation, and a tube treated with C450 shows an increase in biofilm accumulation compared to untreated reference tube. The crystal violet stain from the tubes was dissolved and collected into cuvettes, and this solution was measured for optical density at wavelength 570, (correlating with measurements shown on the bottom of the figure).

FIG. 7 shows a standard hatched bacterial plating technique and our modified outside-in plating technique. The spots indicate the point of addition of the solvent control, the inhibitors, and/or ligand. The line shows the streaking patterns.

FIG. 8 shows the points of interrogation of the quorum sensing pathway in recombinant wild-type proxy V. campbellii strain with cqsS and luxPQ genes inactivated. 1) luxM-strain does not produce ligand. 2) Synthetic ligand, harveyi autoinducer-1 HAI-1 can be added as a treatment. 3) LuxN specific protein inhibitor C450-0730 can be added to inhibit the upstream portion of the signaling pathway. 4) luxN mutant H471A disrupts the phosphorylation dependent aspect of the transduction of the signaling pathway. 5) LuxR specific protein inhibitor QStatin or PTSP can be added to inhibit the downstream elements of the signaling pathway. 6) luxB-strain bacteria do not express a final key downstream target of the signaling pathway, LuxB luciferase.

FIG. 9A shows the results of plating of quantified colony forming units (CFUs) of V. campbellii that are wild-type (unmutated), glmS-single mutant, glmS-thyA-double mutant, glmS-thyA-ΔbioABF (D) triple mutant, and glmS-thyA-ΔbioABFCD hemA-quadruple mutant onto restrictive media. The bacteria were imaged in the light (bright field) and in the dark (luminescent). Wild-type (unmutated) bacteria show no hindrance of growth on restrictive media. Mutant bacteria able to grow on restrictive media are considered escapee CFUs. Many escapee CFUs are visible on glmS-single mutant plates. Fewer escapee CFUs are visible on glmS-thyA-double mutant plates. No escapee CFUs are visible on glmS-thyA-ΔbioABFCD triple mutant, and glmS-thyA-ΔbioABFCD) hemA-quadruple mutant plates.

FIG. 9B shows calculated escape frequencies (CFU count on restrictive plate/total CFU plated) plotted for glmS-single, thyA-single, and glmS-thyA-double mutants. Top of column=average, error bars=+/−standard deviation. glmS-thyA-ΔbioABFCD triple and glmS-thyA-ΔbioABFCD) hemA-quadruple mutants had no escapee CFUs identified (n≥4 for each strain). Thus, no average escape frequency could be established for glmS-thyA-ΔbioABFCD) triple and glmS-thyA-ΔbioABFCD hemA-quadruple mutants. Instead, the escape frequencies shown here depict less than or equal to one divided by the number of total CFU plated in one solid media assay for that strain to establish the maximum predicted escape frequency. Actual escape frequencies for these two strains are predicted to be lower than the values presented here. Escape assays were performed a minimum of four trials per strain. The dotted line represents the acceptable level of biological containment of a recombinant organism, which should be <1 escapee in 10\hat{ }8 organisms, established in the National Institute of Health (NIH) guidelines for research involving recombinant or synthetic nucleic acid molecules, as of April 2024.

FIG. 10 shows co-culturing of recombinant wild-type proxy V. campbellii (WT, top) and recombinant quorum sensing disrupted V. campbellii strain luxN-H471A (N-H471A, bottom). Plates were treated with inhibitors that were resuspended in 91% isopropanol and added on the right side of each plate shown. Solvent control isopropanol was added to the opposing left side. Both were allowed to evaporate/absorb into the agar plate prior to plating of bacteria. 20 μl of LuxN inhibitors were added: A, 20 mM C450-0730; B, 20 mM C646-0078; C, 20 mM 3578-0898; D 20 mM 4248-0174; E, 20 mM Chlorolactone; F, 10 mM N-Dodecanoyl-L-homoserine lactone; G, 10 mM N-Decanoyl-L-homoserine lactone; H N-Octanoyl-L-homoserine lactone; and I, N-(3-Oxododecanoyl)-L-homoserine lactone. Two plates were treated with LuxR inhibitors: 10 μl 5 mM QStatin (J) and 10 μl 1 mM PTSP (K). Also shown is on negative control (N/C) agar plate that was untreated. The bacteria were imaged in the light (bright environment) and in the dark (dark environment). These results are summarized in Table 6.

FIG. 11 shows individual culturing and co-culturing of recombinant wild-type proxy V. campbellii (WT) and recombinant quorum sensing disrupted V. campbellii daughter strains luxM-, luxB-, and luxN-H471A (N—H471A). The top row shows strains that are plated individually as single cultured strains. Plates marked 1, 2, 3, 4, and 5 were treated with additives that were resuspended in 91% isopropanol and added on the right side of each plate shown. Solvent control isopropanol of matching volume was added to the opposing left side. Both were allowed to evaporate/absorb into the agar plate prior to plating of bacteria. Rows 2-6 show plates with two strains co-cultured on each plate: A, WT (top) & LuxB-(bottom); B, WT (top) & LuxM-(bottom), C, LuxB-(top) & LuxM-(bottom); and D, LuxN-H471A (top) & LuxM-(bottom). The seventh row (HAI-1) shows plates with LuxM-plated both on the top and bottom of the plates. Rows three and four are treated with LuxR inhibitors: 10 μl 5 mM QStatin (1A-1D) and 10 μl 1 mM PTSP (2A-2D). Rows five and six are treated with LuxN inhibitors: 20 μl of 20 mM C450-0730 (3A-3D) and 20 μl of 20 mM Chlorolactone (4A-4D). The bottom seventh row shows plates that were treated with synthetic ligand HAI-1:10 μl 0.1 mM HAI-1 (5E) and 5 μl 0.1 mM HAI-1 (5F). For E&F, LuxM-strain is on both the top and bottom of each plate. The bacteria were imaged in the light (bright environment) and in the dark (dark environment).

FIG. 12 is a table providing chemical names, CAS numbers and/or SMILES (Simplified Molecular Input Line Entry System) information.

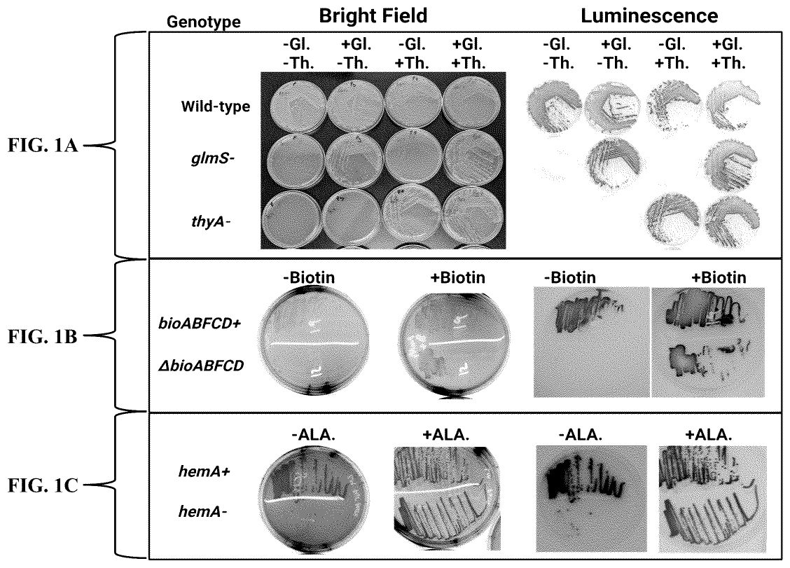

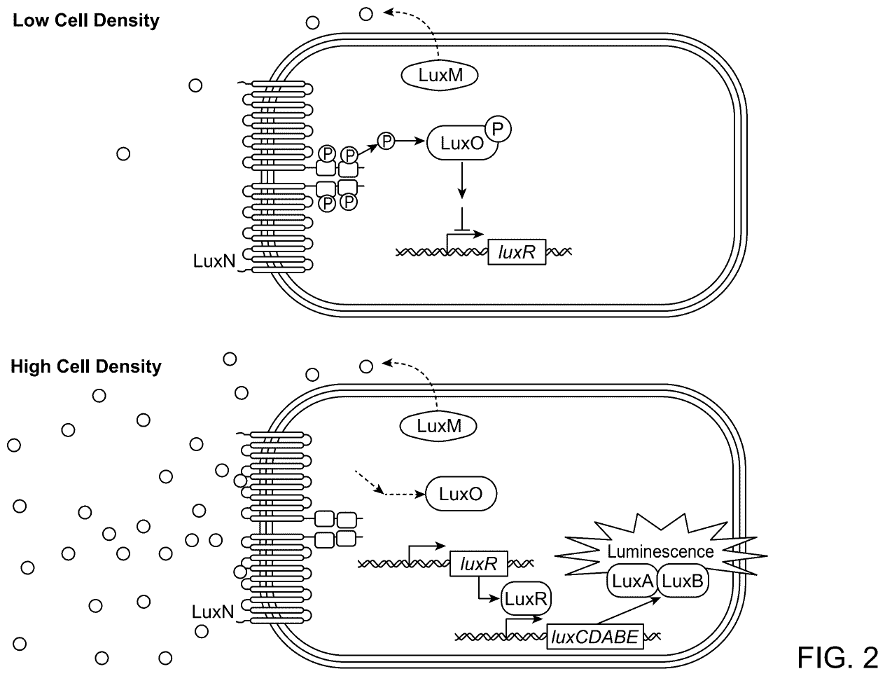

Acknowledgements: Figures created with BioRender.com.

### BRIEF DESCRIPTION OF THE SEQUENCES

SEQ ID NO: 1: nucleotide sequence encoding glmS of V. campbellii.

SEQ ID NO: 2: nucleotide sequence encoding thyA of V. campbellii.

SEQ ID NO: 3: nucleotide sequence encoding cqsS of V. campbellii.

SEQ ID NO: 4: nucleotide sequence encoding luxPQ of V. campbellii.

SEQ ID NO: 5: nucleotide sequence encoding luxN of V. campbellii.

SEQ ID NO: 6: nucleotide sequence encoding luxM of V. campbellii.

SEQ ID NO: 7: nucleotide sequence encoding luxB of V. campbellii.

SEQ ID NO: 8: nucleotide sequence encoding the deleted region from @HAP-1-like bacteriophage region of V. campbellii.

SEQ ID NO: 9: nucleotide sequence encoding the deleted region from Kappa-like bacteriophage region of V. campbellii.

SEQ ID NO: 10: nucleotide sequence encoding bioABFCD of V. campbellii.

SEQ ID NO: 11: nucleotide sequence encoding hemA of V. campbellii.

SEQ ID NO: 12: nucleotide sequence encoding recA of V. campbellii.

SEQ ID NO: 13: nucleotide sequence encoding bioA of V. campbellii.

SEQ ID NO: 14: nucleotide sequence encoding bioB of V. campbellii.

SEQ ID NO: 15: nucleotide sequence encoding bioF of V. campbellii.

SEQ ID NO: 16: nucleotide sequence encoding bioC of V. campbellii.

SEQ ID NO: 17: nucleotide sequence encoding bioD of V. campbellii.

SEQ ID NO: 18: nucleotide sequence encoding luxP of V. campbellii.

SEQ ID NO: 19: nucleotide sequence encoding luxQ of V. campbellii.

SEQ ID NO: 20: nucleotide sequence encoding luxA of V. campbellii.

### DETAILED DISCLOSURE OF THE INVENTION

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. In the context of reagent and/or analyte concentrations, the term “about” can mean a range of up to 0-20%, 0 to 10%, 0 to 5%, or up to 1% of a given value.

The term “and/or” as used herein refers to and encompasses any and all possible combinations of one or more of the associated listed items. For example, the phrase “A, B, and/or C” includes A alone, B alone, C alone, the combination of A and B, the combination of A and C, the combination of B and C, and the combination of A, B, and C. Also, the term “or” is used in its inclusive sense (and not in its exclusive sense) so that when used, for example, to connect a list of items, the term “or” means one, some, or all of the items in the list. Conjunctive language such as the phrase “at least one of X, Y, and Z.” unless specifically stated otherwise, is otherwise understood with the context as used in general to convey that an item, term, etc. may be either X, Y, or Z; X and Y; X and Z; Y and Z; or X, Y, and Z (i.e., any combination of X, Y, and Z).

In the present disclosure, ranges are stated in shorthand, so as to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values.

As used herein, the term “positive,” when referring to a result or signal, indicates the presence of an analyte or item that is being detected in a sample. The term “negative,” when referring to a result or signal, indicates the absence of an analyte or item that is being detected in a sample. Positive and negative are typically determined by comparison to at least one control, e.g., a threshold level that is required for a sample to be determined positive, or a negative control (e.g., a known blank). A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters and will understand which controls are valuable in a given situation and be able to analyze data based on comparisons to control values. Controls are also valuable for determining the significance of data. For example, if values for a given parameter are variable in controls, variation in test samples will not be considered as significant.

As used herein, the term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene. The term “ortholog” or “orthologs” refers to genes in different species of a genus of bacterial cells that have the same or equivalent function. An Ortholog(s) of a gene, for example luxB, can have the same nucleotide or amino acid sequence or a different nucleotide or amino acid sequence from that disclosed herein (e.g., SEQ ID NO: 7) but has the same or equivalent function (i.e., quorum sensing related luminescence).

As used herein, the term “isolated nucleic acid” molecule refers to a nucleic acid molecule that is separated from other nucleic acid molecules that are usually associated with the isolated nucleic acid molecule. Thus, an “isolated nucleic acid molecule” includes, without limitation, a nucleic acid molecule that is free of nucleotide sequences that naturally flank one or both ends of the nucleic acid in the genome of the organism from which the isolated nucleic acid is derived (e.g., a cDNA or genomic DNA fragment produced by PCR or restriction endonuclease digestion). In addition, an isolated nucleic acid molecule can include an engineered nucleic acid molecule such as a recombinant or a synthetic nucleic acid molecule. A nucleic acid molecule existing among hundreds to millions of other nucleic acid molecules within, for example, a nucleic acid library (e.g., a cDNA or genomic library) or a gel (e.g., agarose, or polyacrylamide) containing restriction-digested genomic DNA, is not an “isolated nucleic acid”.

As used herein, the term “gene” means the segment of DNA involved in producing a polypeptide chain; it includes regions preceding and following the coding region (leader and trailer) involved in the transcription/translation of the gene product and the regulation of the transcription/translation, as well as intervening sequences (introns) between individual coding segments (exons).

As used herein, the terms “identical” or percent “identity”, in the context of describing two or more polynucleotide or amino acid sequences, refer to two or more sequences or subsequences that are the same or have a specified percentage of amino acid residues or nucleotides that are the same (for example, a nucleotide probe used in the method of this invention has at least 70% sequence identity, preferably 80%, 85%, 90%, 91%, 92%, 93%, 94%, 95%, 96%, 97%, 98%, 99%, or 100% identity, to a target sequence or complementary sequence thereof), when compared and aligned for maximum correspondence over a comparison window, or designated region as measured using one of the following sequence comparison algorithms or by manual alignment and visual inspection. Such sequences are then said to be “substantially identical”. With regard to polynucleotide sequences, this definition also refers to the complement of a test sequence.

As used herein, the term “recombinant bacterium” refers to a bacterial cell that has been genetically modified from its native state. For instance, a recombinant bacterium may comprise one or more nucleotide insertions, nucleotide deletions, nucleotide rearrangements, and nucleotide modifications. These genetic modifications may be introduced into the chromosome of the bacterium, or alternatively be present on an extrachromosomal nucleic acid (e.g., a plasmid). Recombinant bacteria of the disclosure may comprise a nucleic acid located on a plasmid. Alternatively, the recombinant bacteria may comprise a nucleic acid located in the bacterial chromosome (e.g., stably incorporated therein). In some embodiments, the recombinant bacterium is avirulent. In some embodiments the recombinant bacterium exhibits reduced virulence. In some embodiments, the recombinant bacterium is virulent. In some embodiments, the recombinant bacterium is attenuated (exhibits reduced virulence).

As used herein, a “plasmid” or “vector” includes a nucleic acid construct designed for delivery to a host cell or transfer between different host cells. The nucleic acid incorporated into the plasmid can be operatively linked to flanking sequences of the host cell.

As used herein, the term “exogenous” refers to a substance (e.g., a nucleic acid or polypeptide) present in a cell other than its native source. The term exogenous can refer to a nucleic acid or a protein that has been introduced by a process involving the hand of man into a biological system such as a cell or organism in which it is not normally found or in which it is found in undetectable amounts. A substance can be considered exogenous if it is introduced into a cell or an ancestor of the cell that inherits the substance. In contrast, the term “endogenous” refers to a substance that is native to the biological system or cell.

A “pharmaceutical composition,” as used herein, refers to a composition comprising an active ingredient (e.g., a recombinant bacterium described herein) with other components such as a physiologically suitable carrier and/or excipient.

As used herein, the term “pharmaceutically acceptable carrier” or a “pharmaceutically acceptable excipient” refers to a pharmaceutically-acceptable material, composition or vehicle, such as a liquid or solid filler, diluent, excipient, or manufacturing aid (e.g., lubricant, talc magnesium, calcium or zinc stearate, or steric acid). Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline (e.g., phosphate-buffered saline (PBS)); (18) Ringer's solution; (19) ethyl alcohol; (20) isopropyl alcohol; (21) pH buffered solutions; (22) polyesters, polycarbonates and/or polyanhydrides; (23) bulking agents, such as polypeptides and amino acids (24) serum component, such as serum albumin, HDL and LDL; (25) C2-C12 alcohols, such as ethanol; and (26) other non-toxic compatible substances employed in pharmaceutical formulations. Wetting agents, coloring agents, release agents, coating agents, disintegrating agents, binders, sweetening agents, flavoring agents, perfuming agents, protease inhibitors, plasticizers, emulsifiers, stabilizing agents, viscosity increasing agents, film forming agents, solubilizing agents, surfactants, preservatives and antioxidants can also be present in the formulation. The terms such as “excipient”, “carrier”, “pharmaceutically acceptable excipient” or the like are used interchangeably herein.

### Recombinant Bacteria

The subject invention provides recombinant bacteria that possess genetic mutations that attenuate the bacteria and/or introduce an auxotrophy and alter the quorum sensing pathway. In various embodiments, the bacteria is a Vibrio sp., including, for example, Aliivibrio fischeri, Vibrio campbellii, V. harveyi, V. alginolyticus, V. anguillarum, and V. fluvialis. In other embodiments, the bacteria is Aliivibrio fischeri (formerly Vibrio fischeri). In certain other embodiments, the bacteria is V. harveyi or V. campbellii. In various embodiments, one or more genes in the quorum sensing pathway and one or more genes are mutated such that the recombinant bacteria is altered such that it is an auxotrophic mutant dependent on one or more compounds for growth. In some embodiments, the recombinant bacteria may depend on at least two compounds to regulate the growth and virulence of the bacteria and, by controlling the expression of multiple genes, allow for biological containment. The dependence on multiple compounds for growth and/or virulence enhances the safety of the recombinant bacteria, given the improbability that the organisms will encounter these compounds in a naturally occurring environment.

In certain embodiments, the recombinant bacteria is an auxotrophic mutant for at least 1, 2, 3, 4, 5, 6, 7, 8, 9, 10 or more essential compounds that are prototrophically produced in a corresponding wild-type (unmutated) bacteria. In certain embodiments, the essential compound is glucosamine, biotin, 5-aminolevulinic acid (5-ALA), thymidine, or a combination thereof. In certain embodiments, the glmS locus can be inactivated in the bacterium, whereby glucosamine synthase is not synthesized and fructose-6-phosphate is not converted to glucosamine-6-phosphate. In certain embodiments, the thyA locus can be inactivated, whereby thymidylate synthase is not synthesized and deoxyuridine monophosphate (dUMP) is not converted into deoxythymidine monophosphate (dTMP). In yet other embodiments, one or more of the genes associated with biotin synthesis can be inactivated or deleted (e.g., bioA, bioB, bioF, bioC, and/or bioD can be inactivated or deleted within the bacteria). In some embodiments, the entire biotin locus (bioABFCD)) can be inactivated or deleted from the genome of the bacterial cell and, in other embodiments, one or both of bioA and/or bioß can be inactivated. Yet other embodiments provide for the inactivation or deletion of the hemA gene in a bacterial cell which results in the bacterial cell being unable to express a glutamyl tRNA reductase, an essential enzyme for synthesis of aminolevulinic acid, a precursor for heme biosynthesis. The inactivation of the hemA gene requires supplementation of 5-aminolevulinic acid to facilitate bacterial growth and survival. In yet other embodiments, the recA gene is inactivated or deleted such that recombinational DNA repair is impaired within the bacterial cell.

In various embodiments, a single gene or more than one gene can be inactivated to create an auxotrophic mutant. Thus, one or more of glmS, thyA, bioABFCD, and/or hemA can be inactivated to create an auxotrophic mutant.

The bacterial cells may further comprise the inactivation or deletion of the recA gene. Table 1 illustrates various gene combinations that can be inactivated in order to create an auxotrophic mutant as disclosed herein that further comprises inactivation or deletion of the recA gene.

Additionally, one or more of bioA, bioB, biof, bio (′, and/or biol) can be inactivated or deleted or the entire bioABFCD) operon can be inactivated in the creation of the auxotrophic mutant. For the purposes of Table 1, the bioABFCD) designation is intended to include each individual gene of the bioABFCD operon (bioA, bioB, bioF, bioC, and/or bioD) as well as the entire operon for the purposes of inactivation to introduce an auxotrophic mutation within the bacterium. In certain embodiments, the auxotrophic mutant is glmS−, thyA−, ΔbioABFCD, and further comprises inactivation or deletion of recA−. In other words, the auxotrophic mutant contains mutations that inactivate or delete glmS, thyA, bioABFCD) to create the auxotrophic mutant, and recA.

In certain embodiments, the recombinant bacteria further comprise modifications that reduce or inhibit the ability for a bacteriophage that is integrated into the bacterial chromosome to form. For example, modifications of DNA encoding phage capsid proteins and/or tail proteins can be included in the disclosed recombinant bacterial cells (e.g., such as deletions within or insertions within the DNA encoding phage capsid proteins and/or tail proteins). In certain embodiments, the bacterium comprises a mutation or insertion in the @HAP-1-like bacteriophage region as disclosed herein (e.g., SEQ ID NO: 8) or a deletion of the @HAP-1-like bacteriophage region or any portion thereof. In certain embodiments, the bacterium comprises a mutation or insertion in the Kappa-like phage region (e.g., SEQ ID NO: 9) or a deletion of Kappa-like phage region or any portion thereof as disclosed herein. In preferred embodiments, the bacterium comprises a mutation in the @HAP-1-like bacteriophage region and the Kappa-like phage region. In other embodiments, any chromosomal sequence resembling a phage capsid and/or tail region can be modified such that the bacteriophage cannot form.

In certain embodiments, the recombinant bacteria also comprise modifications that alter the quorum sensing pathway. In certain embodiments, the recombinant bacteria cannot sense cholera autoinducer-1 (CAI-1), autoinducer-2 (AI-2), harveyi autoinducer-1 (HAI-1), or any combination thereof. In certain embodiments, the bacterium possesses a deletion, mutation, or insertion in the cqsS gene, the luxPQ genes, the luxN gene, the luxM gene, the luxA gene, the luxB gene, or any combination thereof that decreases or eliminates expression of the gene or any combination of genes or orthologs thereof. In various embodiments, the cqsS gene, the luxPQ genes, the luxN gene, the luxM gene, the luxA gene, the luxB gene can contain mutations that alter the function of the enzyme or protein expressed by the gene (e.g., the LuxN-H471A mutation described herein). In certain embodiments, the bacterium comprises a deletion in the cqsS gene, the luxPQ genes, the luxN gene, the luxM gene, the luxA gene, the luxB gene, any orthologs thereof, or any combination thereof that decreases or eliminates expression of the gene or any combination of genes. In preferred embodiments, the bacterium comprises a deletion or mutation in the cqsS gene and the luxPQ genes or any orthologs thereof (e.g., is CqsS− and LuxPQ−).

In certain embodiments, the recombinant bacterial strains can be provided in a kit in combination with instructions for using the kit components. In further embodiments, the kit comprises prepared media or the components to synthesize the media for growing the bacteria. In one embodiment, the media is supplemented with thymidine and glucosamine.

The instant disclosure further provides recombinant bacteria with desirable safety features that can be safely used to effectively deliver antigenic compounds to a subject (e.g., fish or shellfish, such as shrimp) in order to mount potent immunogenic responses against pathogens, such as Vibrio harveyi, V. anguillarium, V. vulnificus, or other pathogenic Vibrio spp.

In certain embodiments, the bacterium comprises a mutation or insertion in the glmS gene or a deletion of the glmS gene (SEQ ID NO: 1) or any portion thereof that decreases or eliminates expression of glmS. In certain embodiments, the mutation can be a single point mutation or deletion or at least 2, about 5, about 10, about 50, about 100, about 250, about 500, about 1000, about 1500, about 1600, about 1700, about 1800, or about 1833 nucleotides encoding glmS can be mutated or deleted. In various embodiments, the deleted nucleotides can be fewer than the entire glmS coding sequence in order to minimize the effects of the mutation on neighboring genes. For example, 9 to 50 nucleotides at the 5′ and 3′ ends of the coding sequence can be retained in order to minimize the effects of the mutation on neighboring genes.

In certain embodiments, the bacterium comprises a mutation or insertion in the thyA gene or a deletion of the thyA (SEQ ID NO: 2) gene or any portion thereof that decreases or eliminates expression of thyA. In certain embodiments, the mutation can be a single point mutation or deletion or at least 2, about 5, about 10, about 50, about 100, about 250, about 500, about 750, about 800, or about 852 nucleotides encoding thyA can be mutated or deleted. In various embodiments, the deleted nucleotides can be fewer than the entire thyA coding sequence in order to minimize the effects of the mutation on neighboring genes. For example, 9 to 50 nucleotides at the 5′ and 3′ ends of the coding sequence can be retained in order to minimize the effects of the mutation on neighboring genes. In certain embodiments, the bacterium comprises a mutation or deletion in the glmS gene, the bioABFCD genes and the thyA gene.

In certain embodiments, the recombinant bacteria comprise modifications that reduce or inhibit the ability for a bacteriophage to form. In certain embodiments, the bacterium comprises a mutation or insertion in the @-HAP-1-like bacteriophage region (e.g., Hap9427; SEQ ID NO: 8) or a deletion of the @-HAP-1-like bacteriophage region or any portion thereof. In certain embodiments, the bacterium comprises a mutation or insertion in the Kappa-like phage region (e.g., Kap10060; SEQ ID NO: 9) or a deletion of Kappa-like phage region or any portion thereof. In certain embodiments, the bacterium comprises a mutation in the @-HAP-1-like bacteriophage region and the Kappa-like phage region. In certain embodiments, the mutation can be a point mutation or deletion or at least 2, about 5, about 10, about 50, about 100, about 1000, about 2500, about 5000, about 8000, or about 9427 nucleotides encoding Φ-HAP-1-like bacteriophage region can be mutated or deleted. In certain embodiments, the mutation can be a point mutation or at least 2, about 50, about 100, about 500, about 1000, about 2500, about 5000, about 7500, or about 10060 nucleotides encoding Kappa-like phage region can be mutated or deleted.

In certain embodiments, the recombinant bacteria comprise modifications that alter the quorum sensing pathway, including, for example, the inactivation of at least one gene in the quorum sensing pathway. In certain embodiments, the recombinant bacteria cannot sense cholera autoinducer-1 (CAI-1). In certain embodiments, the bacterium comprises a mutation or insertion in the cqsS gene (SEQ ID NO: 3) or a deletion of cqsS gene or any portion thereof that decreases or eliminates expression of cqsS. In certain embodiments, the recombinant bacteria cannot sense autoinducer-2 (AI-2). In certain embodiments, the bacterium comprises a mutation or insertion in the luxPQ (SEQ ID NO: 4) genes or a deletion of the luxPQ genes or any portion thereof that decreases or eliminates expression of luxPQ). In certain embodiments, the recombinant bacteria cannot sense harveyi autoinducer-1 (HAI-1). In certain embodiments, the bacterium comprises a mutation or insertion in the luxN gene (SEQ ID NO: 5) or a deletion of the luxN gene or any portion thereof that decreases or eliminates expression of luxN. For example, an H471 mutation (e.g., H471A) can be introduced into luxN that alters the function of the protein but not its expression. In certain embodiments, the bacterium comprises a mutation or insertion in the luxM gene (SEQ ID NO: 6) or a deletion of the luxM gene or any portion thereof that decreases or eliminates expression of luxM. In certain embodiments, the bacterium comprises a mutation or insertion in the luxB gene (SEQ ID NO: 7) or a deletion of the luxB gene or any portion thereof that decreases or eliminates expression of luxB. In one embodiment, the bacterium comprises point mutations in the luxN gene that produce a protein that has an H471A missense mutation within the protein. In certain embodiments, the recombinant bacteria cannot sense CAI-1 or AI-2, CAI-1 or HAI-1, or HAI-1 and AI-2. In one embodiment, the recombinant bacteria cannot sense CAI-1 or AI-2.

In various embodiments, the bacterium comprises inactivation of the cqsS gene and the luxPQ genes. In some embodiments, the bacterium comprises an inactivation in the cqsS gene, the luxN gene, and the luxPQ genes. In other embodiments, the bacterium comprises an inactivation in the cqsS gene, the luxM gene, and the luxP′ (genes. In yet other embodiments, the bacterium comprises a mutation in the cqsS gene, the luxB gene, and the luxPQ genes. In additional embodiments, the bacterium comprises the inactivation of luxA, luxB, luxM, or luxN alone or in any combination and/or in combination with the inactivation of the cqsS gene.

In certain embodiments, the mutation can be a point mutation or deletion or at least 2, about 5, about 10, about 50, about 100, about 250, about 500, about 1000, about 1500, or about 2061 nucleotides encoding cqsS the gene can be mutated or deleted. In various embodiments, the deleted nucleotides can be fewer than the entire cqsS coding sequence in order to minimize the effects of the mutation on neighboring genes. For example, 9 to 50 nucleotides at the 5′ and 3′ ends of the coding sequence can be retained in order to minimize the effects of the mutation on neighboring genes.

In certain embodiments, the mutation can be a point mutation or deletion or at least 2, about 5, about 10, about 100, about 250, about 500, about 1000, about 2000, about 3000 or about 3678 nucleotides encoding luxPQ can be mutated or deleted. In various embodiments, the deleted nucleotides can be fewer than the entire luxP and/or luxQ coding sequence in order to minimize the effects of the mutation on neighboring genes. For example, 15 to 58 nucleotides at the 5′ and 3′ ends of the coding sequence can be retained in order to minimize the effects of the mutation on neighboring genes.

In certain embodiments, the mutation can be a point mutation or deletion or modification of at least 2, 3, (e.g., H471A), about 5, about 10, about 50, about 100, about 250, about 500, about 1000, about 1500, about 2000 or about 2550 nucleotides encoding luxN the gene can be mutated or deleted. In various embodiments, the deleted nucleotides can be fewer than the entire luxN coding sequence in order to minimize the effects of the mutation on neighboring genes. For example, 10 to 50 nucleotides at the 5′ and 3′ ends of the coding sequence can be retained in order to minimize the effects of the mutation on neighboring genes.

In certain embodiments, the mutation can be a point mutation or deletion or at least 2, about 5, about 10, about 50, about 100, about 250, about 500, about 750, about 1000 or about 1200 nucleotides encoding luxM the gene can be mutated or deleted. In various embodiments, the deleted nucleotides can be fewer than the entire luxM coding sequence in order to minimize the effects of the mutation on neighboring genes. For example, 10 to 50 nucleotides at the 5′ and 3′ ends of the coding sequence can be retained in order to minimize the effects of the mutation on neighboring genes.

In certain embodiments, the mutation can be a point mutation or deletion or at least 2, about 5, about 10, about 50, about 100, about 250, about 500, about 750, or about 975 nucleotides encoding luxB the gene can be mutated or deleted. In various embodiments, the deleted nucleotides can be fewer than the entire luxB coding sequence in order to minimize the effects of the mutation on neighboring genes. For example, 10 to 50 nucleotides at the 5′ and 3′ ends of the coding sequence can be retained in order to minimize the effects of the mutation on neighboring genes.

In certain embodiments, the mutation can be a point mutation or deletion or at least 2, about 5, about 10, about 50, about 100, about 250, about 500, about 750, about 900, or about 1000 nucleotides encoding luxA the gene can be mutated or deleted. In various embodiments, the deleted nucleotides can be fewer than the entire luxB coding sequence in order to minimize the effects of the mutation on neighboring genes. For example, 10 to 50 nucleotides at the 5′ and 3′ ends of the coding sequence can be retained in order to minimize the effects of the mutation on neighboring genes.

In certain embodiments, one or more endogenous genes are deleted from the bacterial chromosome. In certain embodiments, the deletion is a partial deletion of the endogenous gene. In certain embodiments, the deletion is a full-length deletion of the endogenous gene. In certain embodiments, the gene is genetically-altered to prevent transcription and/or translation of the gene encoding the protein, such as, for example, mutating or deleting the promoter region of the gene. In certain embodiments, the endogenous gene is genetically altered to insert a transcriptional terminator in the open reading frame of the gene. In certain embodiments, a regulatory region of the gene is genetically-modified to alter the expression of the gene. With respect to the phage sequences, these sequences are integrated into the chromosomes contained within the bacterium. As discussed above, certain elements of the phage sequences can be inactivated in order to reduce or eliminate the ability of the bacterium to produce active phage.

The terms “inactivate”, “inactivated” and “inactivating” are used to indicate the introduction of genetic alterations or mutations into a gene that inhibit or abolish the expression of an active protein that is encoded by the gene. Typically, inactivating a gene in a bacterial cell comprises introducing into the gene one or more alterations or mutations of the gene that inhibit or abolish the expression of an active protein from the gene. Alterations or mutations in a gene that inhibit or abolish the expression of a protein from the gene can be achieved either by deleting the entire coding region of the gene, deleting a portion of the coding region of the gene, by introducing a frame shift mutation within the coding region of the gene, by introducing a missense mutation or a point mutation, insertion of sequences that disrupt the activity of the protein encoded by the gene (e.g., by introducing a stop codon into the coding region of the gene), or any combination of the aforementioned alterations of the gene (which may also be referred to as a “mutation of a/the gene” or “mutations of a/the gene”). Inactivating a gene can also be performed by using molecular markers, such as genes encoding antibiotic resistance or a fluorescent protein, such as GFP.

Methods of inactivating a gene of interest in a bacterial cell to inhibit or abolish the expression of an active protein from the gene are well known to a person of ordinary skill in the art and such embodiments are within the purview of the invention. Certain such embodiments are identified below.

In certain embodiments, deletion mutations (alterations) are generated with and without insertions using suicide vector (i.e., bacterial allelic exchange vector) technologies (see Edwards et al. (1998) Gene 207:149-57 (195); Kaniga et al. (1998) Infect. Immun. 66:5599-606 (196); Maloy and Nunn (1981) J. Bacteriol. 145:1110-2 (197); Miller and Mekalanos (1988) J. Bacteriol. 170:2575-83 (198); Ried and Collmer (1987) Gene 57:239-46 (199); and Roland et al. (1999) Avian Dis. 43:429-41 (200)). Suicide vectors having flanking sequences derived from the host cell (e.g., V. campbellii) for each of genes or regions described herein, including for example, glmS, thyA, cqsS, luxPQ, luxN, luxM, luxB, the @-HAP-1-like bacteriophage region, and the Kappa-like prophage region can be used to introduce deletions within these genes (introduce deletions mutations). The suicide vector allows for a nucleotide sequence of the suicide vector to homologously recombine into the chromosome of the bacterial cell at a location matching the sequences of the flanking regions of the suicide vector. Commercially available vectors available for use to alter the nucleotide sequence encoding genes or promoters thereof include, for example, pCM433, pPS04, pT18mobsacBm, or pDESTIK18 ms. These gene alterations can be introduced using either transformation of suicide vectors followed by selection or by conjugational transfer of suicide vectors using standard methods. Plasmid constructs can be evaluated by DNA sequencing.

In certain embodiments, inactivating a gene of interest is performed using the CRISPR/Cas system. Typically, a CRISPR/Cas system-mediated inactivation of a gene involves the use of a guide RNA targeted to a gene of interest. A DNA oligomer targeted to a gene of interest can be transcribed into single guide RNA (sgRNA). sgRNA guides the Cas9 DNA endonuclease to the gene of interest by sgRNA hybridization to the target site. Based on the sequence of glmS, bioABFCD operon (or bioA, bioB, biof, bioC, or biol) genes individually), hemA, recA, thyA, cqsS, luxPQ, luxN, luxM, luxB, chromosomal regions containing phage sequences, such as the Φ-HAP-1-like bacteriophage region (SEQ ID NO: 8) and Kappa-like prophage region (SEQ ID NO: 9 as disclosed herein), a person of ordinary skill in the art can design and perform inactivation of the gene using the CRISPR/Cas system and such embodiments are within the purview of the invention.

Methods of inactivating a gene of interest in a bacterial cell to inhibit or abolish the expression of an active protein from the gene also include introduction into the bacterial cell one or more inhibitory oligonucleotides, such as small interfering RNA (siRNA) or short hairpin RNAs (shRNA). Methods of producing and introducing inhibitory RNA are also well known to a person of ordinary skill in the art and such embodiments are within the purview of the invention.

The invention further provides kits, including bacteria, packaged into suitable packaging material, optionally in combination with instructions for using the kit components, e.g., instructions for performing a method of the invention. In one embodiment, a kit includes an amount of bacteria and instructions for running the assay on a label or packaging insert. In further embodiments, a kit includes an article of manufacture for performing the assay. In further embodiments, the kit comprises prepared media or the components to synthesize the media for growing the bacteria. In certain embodiments, the media can be Luria Marine agar and broth. The bacteria in the kit can be provided in lyophilized form, in a frozen sample, in a liquid media, or on a solid media. In certain embodiments, the media or the components to synthesize the media can comprise glucosamine, biotin, 5-ALA, and/or thymidine. The kits contain at least one, two, three, four, or more of the mutated bacterial cells disclosed herein (such as those disclosed in Table 4).

In a preferred embodiment, the kit according to the invention can also contain further reagents suitable for growing a bioluminescent bacterium, including, for example, an autoinducer. In certain embodiments, the autoinducer is CAI-1, AI-2, HAI-1, or any combination thereof. In preferred embodiments, the included autoinducer is HAI-1. In certain embodiments, the autoinducer can be dissolved in isopropyl alcohol, ethanol, or water that is included in the subject kits or provided by the user.

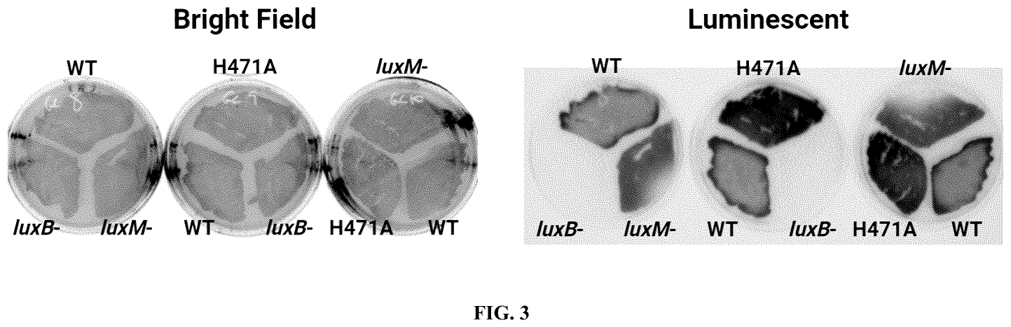

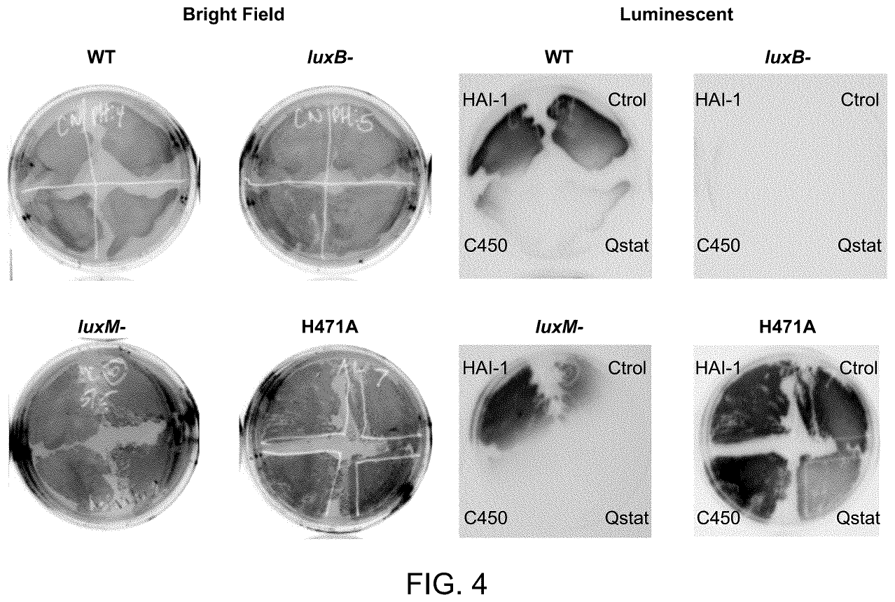

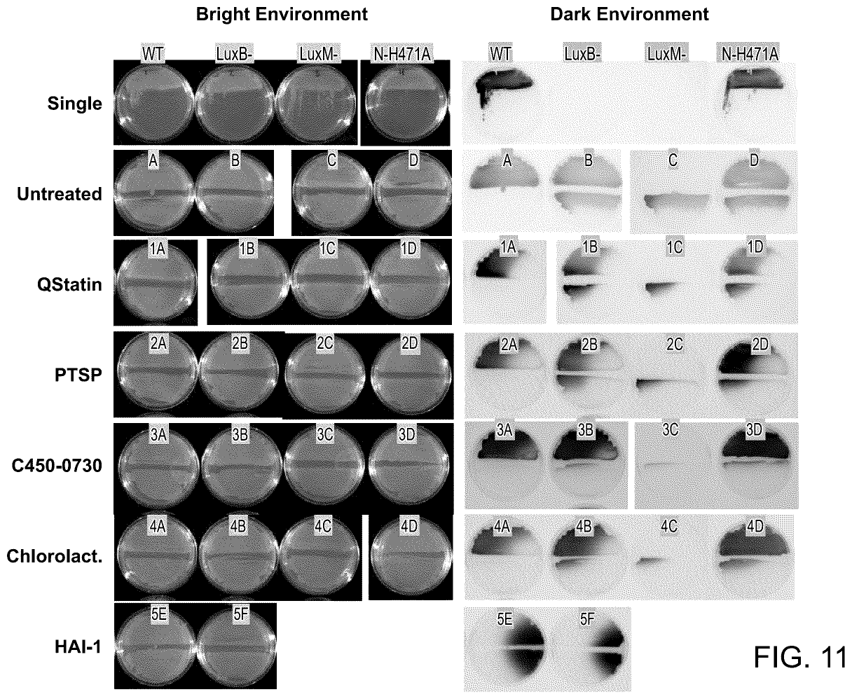

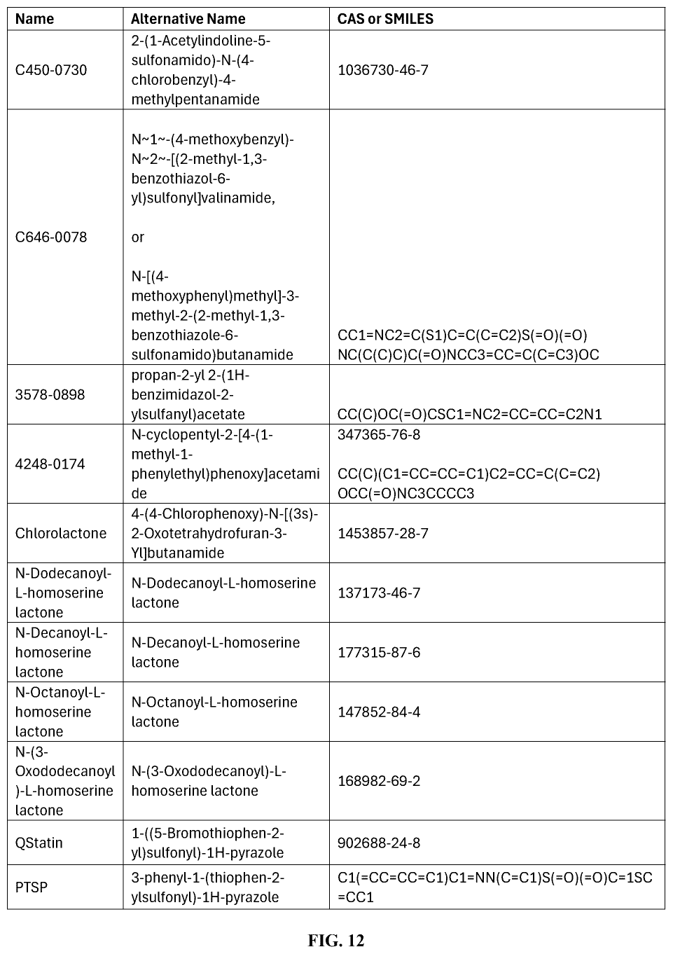

In preferred embodiments, the kit further comprises inhibitors of at least one protein of the quorum sensing pathway, such as, for example, a LuxN inhibitor (e.g., chlorolactone (disclosed in U.S. Pat. No. 9,084,773 B2, which is hereby incorporated by reference in its entirety) or C450-0730), a LuxR inhibitor (e.g., 3-phenyl-1-(thiophen-2-ylsulfonyl)-1H-pyrazole (PTSP) or Qstatin [1-(5-bromothiophene-2-sulfonyl)-1H-pyrazole]), or a combination thereof. Various LuxN and LuxR inhibitors are also disclosed in US2011/0123586A1 and U.S. Pat. Nos. 8,568,756; 9,045,476; and 9,41,040 (each of which is hereby incorporated by reference in its entirety), see FIGS. 3 and 11 and the disclosure related to these compounds/inhibitors in each of these documents. In certain embodiments, the LuxN inhibitor and the LuxR inhibitor can be dissolved in isopropyl alcohol or ethanol that is included in the subject kits or provided by the user.

In another preferred embodiment, the kit according to the invention comprises protein signaling controls. Such controls are known in the art, and can include qualitative controls, positive controls, negative controls, internal controls, quantitative controls, internal quantitative controls, as well as calibration ranges. In certain embodiments, compounds that inhibit or antagonize the quorum sensing pathway of the recombinant bacteria can be included in the kit, such as, for example LuxN. In preferred embodiments, the inhibitor or antagonist is chlorolactone and/or Qstatin and one or both can be included in the kit. In preferred embodiments, the bacterium that comprises a mutation in the cqsS gene, the luxN gene, and the luxPQ gene can be used as negative control, in which the bacterium has interrupted luminescence response. In preferred embodiments, the bacterium that comprises a mutation in the cqsS gene, the luxM gene, and the luxPQ gene can be used as a negative control, in which the bacterium has interrupted ligand synthesis; HAI-1 expressing cells are able to rescue the luminescence of luxM deficient mutants and luxM deficient mutant luminescence can be rescued with commercially producible HAI-1 ligand. In preferred embodiments, the bacterium that comprises a mutation in the cqsS gene, the luxB gene, and the luxPQ gene can be used as a negative control, in which the bacterium has interrupted luminescence.

As used herein, the terms “inhibit”, “inhibition”, “inhibiting”, “antagonist”, “inhibitor”, “antagonize” refer to the reduction or suppression of a given activity for a protein, such as LuxN and/or LuxR. Thus, in some embodiments, these terms indicate a reduction in LuxN activity relative to a control value where no inhibitor or antagonist is present) and indicate a reduction of at least about ten percent relative to a control value. The activity can suppressed/reduced by about 50% compared to a control value, by about 75%, by about 95%, by about 95%, by about 95%, by about 95%, by about 99%, or by 100% as compared to a control value. In some embodiments, the activity can suppressed/reduced by 50% compared to a control value, by 75%, by 95%, by 95%, by 95%, by 95%, by 99%, or 100% as compared to a control value.

In a preferred embodiment, the kit according to the invention contains instructions for the use thereof. Said instructions can advantageously be a leaflet, a card, or the like. Said instructions can also be present under two forms: a detailed one, gathering exhaustive information about the kit and the use thereof, possibly also including literature data; and a quick-guide form or a memo, e.g., in the shape of a card, gathering the essential information needed for the use thereof. Instructions can therefore include instructions for practicing any of the methods of the invention described herein. For example, compositions can be included in a container, pack, or dispenser together with instructions for performing the protein signaling assay. Instructions may additionally include storage information, expiration date, or any information required by regulatory agencies. The instructions may be on “printed matter,” e.g., on paper or cardboard within the kit, on a label affixed to the kit or packaging material, or attached to a vial or tube containing a component of the kit. Instructions may comprise voice or video tape and additionally be included on a computer readable medium, such as a disk (hard disk), a remote server or other online source that is accessed to obtain the instructions, video or other information, optical CD such as CD- or DVD-ROM/RAM, magnetic tape, flash storage, electrical storage media such as RAM and ROM and hybrids of these such as magnetic/optical storage media.

### Method of Immunizing Marine Organisms

In certain embodiments, the disclosed recombinant bacteria can be safely used to effectively deliver antigenic compounds to a marine, brackish water, or freshwater organism (e.g., fish, oysters, shellfish, etc.) in order to mount potent immunogenic responses against pathogens, such as Vibrio spp. The organisms may also be found in brackish water where freshwater and marine water mix. These strains deliver multiple conserved protective Vibrio spp. (e.g., V. campbellii, V. vulnificus, or V. harveyi) surface secreted antigens to induce protective immunity.

In some embodiments, the recombinant bacterium (e.g., V. campbellii, V. vulnificus, or V. harveyi) comprise one or more alterations to the quorum sensing pathway, such as the inactivation of one or more of the genes within the quorum sensing pathway (e.g., one or more of cqsS, luxP, luxQ, luxN, luxA, and luxB). Various combinations of inactivated quorum sensing genes are set forth in Table 2. Additionally, one or more virulence factors can be inactivated if desired. Thus, in one embodiment, recombinant bacteria (e.g., V. campbellii, V. vulnificus, or V. harveyi) for use as a vaccine or immunogen for marine, brackish water, or freshwater organisms can have mutations in the quorum sensing pathway that include inactivation of the cqsS, luxPQ, and luxM genes and, optionally, one or more virulence factors within the recombinant bacteria. In other embodiments, the recombinant bacterium comprises one or more of the bacterial cells/strains provided for in the “Recombinant bacteria” section discussed above, such as Aliivibrio fischeri (formerly V. fischeri), Vibrio campbellii, V. harveyi, V. alginolyticus, V. anguillarum, V. fluvialis and combinations thereof. Some embodiments permit for the use of one or more of the recombinant bacterial strains (e.g., V. campbellii, V. vulnificus, or V. harveyi) that comprise one or more alterations to the quorum sensing pathway, such as the inactivation of one or more of the genes within the quorum sensing pathway (e.g., one or more of cqsS, luxP, luxQ, luxN, luxA, and luxB) and inactivation of one or more essential genes to create an auxotroph (e.g., glmS, thyA, bioABFCD, and hemA) and one or more of the various recombinant bacterial strains discussed in the “Recombinant bacteria” section provided above. For the purposes of this application, a bacterial strain is one in which one of the genetic mutations disclosed herein has been introduced.

In certain embodiments, a pharmaceutical composition comprises the recombinant bacterium disclosed herein and a pharmaceutically acceptable carrier. In certain embodiments, disclosed herein is a method for inducing protective immunity in a marine, brackish water, or freshwater organism, the method comprising administering to the marine, brackish water, or freshwater organism an effective amount of a pharmaceutical composition disclosed herein.

In certain embodiments, the recombinant bacteria can be administered as a treatment prior to infection to protect against disease from Vibrio spp. In certain embodiments, the recombinant bacteria can be directly administered to a juvenile marine, brackish water, or freshwater organism.

A recombinant bacterium may be administered to a subject as a pharmaceutical composition. In some embodiments, the pharmaceutical composition may be used as a vaccine to elicit an immune response to the recombinant bacterium, including any antigens that may be synthesized and delivered by the bacterium. In an exemplary embodiment, the immune response is protective. Immune responses to antigens are well studied and widely reported.

In some embodiments, the pharmaceutical composition comprises a recombinant bacterium described herein. In some embodiments, the pharmaceutical composition comprises a recombinant bacterium that synthesizes a Vibrio antigen of interest. In some embodiments, the pharmaceutical composition comprises at least one, at least two, at least three, at least four, at least five, at least six, at least seven, at least eight, at least nine or more recombinant bacterial strains, as described herein.

Pharmaceutical compositions may be administered to any subject capable of mounting an immune response. Such subjects may include all vertebrates or invertebrates, for example, marine organisms, including laboratory animals. In certain embodiments, the recombinant bacteria can be administered to marine animals, including, for example, a coral, oyster, prawn, shrimp, lobster, common snook, barramundi, turbot, milkfish, brown spotted grouper, sole, sea perch, short sunfish, catfish, and seahorses. As relates to the use of the disclosed bacterial cells as a vaccine, the terms “subject”, “marine, brackish water, or freshwater organism”, and “mariculture animal” can be used interchangeably.

The pharmaceutical composition can be administered to the subject as a prophylactic or for treatment purposes. In some embodiments, the pharmaceutical composition can be administered for the prophylaxis or treatment of blindness, gastro-enteritis, necrotizing enteritis, nodules on the opercula, scale drop and muscle necrosis, skin ulcers, tail rot, and/or vasculitis caused by an infection by a Vibrio spp.

In some embodiments, the recombinant bacterium is alive when administered to a subject in a pharmaceutical composition described herein. In some embodiments, the recombinant bacterium is inactivated when administered to a subject in a pharmaceutical composition described herein. Suitable vaccine composition formulations and methods of administration are detailed below.

A pharmaceutical composition comprising a recombinant bacterium may optionally comprise one or more possible additives, such as carriers, preservatives, stabilizers, adjuvants, and other substances.

In one embodiment, the pharmaceutical composition comprises an adjuvant. Adjuvants are optionally added to increase the ability of the vaccine to trigger, enhance, or prolong an immune response. In exemplary embodiments, the use of a live or inactivated attenuated recombinant bacterium may act as a natural adjuvant. In some embodiments, the recombinant bacterium synthesizes and secretes an immune modulator. Additional materials, such as cytokines, chemokines, and bacterial nucleic acid sequences naturally found in bacteria, like CpG, are also potential vaccine adjuvants.

In some embodiments, the pharmaceutical composition comprises buffered saline (e.g., phosphate-buffered saline (PBS)).

In some embodiments, the pharmaceutical composition comprises a feed product.

In another embodiment, the pharmaceutical may comprise a pharmaceutical carrier (or excipient). Such a carrier may be any solvent or solid material for encapsulation that is non-toxic to the inoculated subject and compatible with the recombinant bacterium. A carrier may give form or consistency, or act as a diluent. Suitable pharmaceutical carriers may include liquid carriers, such as normal saline and other non-toxic salts at or near physiological concentrations, and solid carriers not used for humans, such as talc or sucrose, or animal feed. Carriers may also include stabilizing agents, wetting and emulsifying agents, salts for varying osmolarity, encapsulating agents, buffers, and skin penetration enhancers. Carriers and excipients as well as formulations for parenteral and nonparenteral drug delivery are set forth in Remington's Pharmaceutical Sciences 19th Ed. Mack Publishing (1995).

In some embodiments, the pharmaceutical composition is delivered to a mariculture animal (e.g., fish or marine invertebrate). In some embodiments, the pharmaceutical composition is delivered as an orally administered liquid (e.g., for use in hatcheries for delivery to fish or marine invertebrates). In some embodiments, the pharmaceutical composition is delivered in the water as an immersion vaccination or direct injection into the marine organism.

In certain embodiments, stabilizers, such as lactose or monosodium glutamate (MSG), may be added to stabilize the pharmaceutical composition against a variety of conditions, such as temperature variations or a freeze-drying process. The recombinant bacterium may also be co-administered with glucosamine and/or thymidine as described herein.

The dosages of a pharmaceutical composition can and will vary depending on the recombinant bacterium and the intended subject, as will be appreciated by one of skill in the art. Generally speaking, the dosage need only be sufficient to elicit a protective immune response in a majority of subjects. Routine experimentation may readily establish the required dosage. Typical initial dosages of vaccine for oral administration could be about 1×10⁷ to 1×10¹⁰ CFU or about 1×10⁷ to 1×10¹⁵ CFU depending upon the age of the subject to be immunized and/or the volume of water to which the vaccine is administered. For example, the vaccine can be administered to a tank or other holding system containing one or more species of mariculture animal. Administering multiple doses of the vaccine may also be used as needed to provide the desired level of protective immunity.

In preferred embodiments, oral administration or immersion vaccination are used, although other methods of administering the recombinant bacterium, such as intravenous, intramuscular, subcutaneous injection, or other parenteral routes, are possible. In some embodiments, these compositions are formulated for administration by injection (e.g., intraperitoneally, intravenously, subcutaneously, intramuscularly, etc.).

In another embodiment, the disclosure provides a method for eliciting an immune response against an antigen in a mariculture animal. The method comprises administering to the subject an effective amount of a pharmaceutical composition comprising a recombinant bacterium described herein.

In still another embodiment, a recombinant bacterium may be used in a method for eliciting an immune response against a pathogen in a mariculture animal in need thereof. The method comprises administrating to the mariculture animal an effective amount of a pharmaceutical composition comprising a recombinant bacterium as described herein. In a further embodiment, a recombinant bacterium described herein may be used in a method for ameliorating one or more symptoms of an infectious disease in a mariculture animal in need thereof. The method comprises administering an effective amount of a pharmaceutical composition comprising a recombinant bacterium as described herein.

### Bacterial Plating

In certain embodiments, one or more chemicals can be added to the surface of solid media. In certain embodiments, the chemical, such as, for example, a LuxN inhibitor, a LuxR inhibitor, or HAI-1 can be in liquid form, such as, for example, dissolved in water or alcohol. In certain embodiments, after the application of the liquid chemical, the chemical is allowed to dry. In certain embodiments, bacteria or other microorganisms are then added on top of the dried chemical. In various embodiments, the bacteria or microorganisms are grown in a liquid medium (e.g. a broth) and then added to the surface of the solid media comprising a chemical (e.g., by streaking on the surface of the solid media).

In certain embodiments, the bacteria or other microorganisms can be spread out on the surface of the media using a streaking pattern that is distinct from the standard hatched pattern. In certain embodiments, the recombinant bacteria are directly streaked back and forth across the plate, over where the (dried) chemical has been applied. In certain embodiments, the novel streaking pattern is a decreasing concentric circle pattern, in which each revolution of the streak has a decreased radius and circumference of the circle relative to the previous revolution around a point. In certain embodiments, the outside of the plate containing the media is marked with a dot to indicate where the chemical will be dropped and the external edges of where the bacteria is to be streaked. In certain embodiments, one or more chemicals is applied onto one side of the media, above where the plate has been marked. In certain embodiments, less than: about 75%, about 70%, about 65%, about 60%, about 55%, about 50%, about 45%, about 40%, about 35%, about 30%, about 25%, about 20%, about 15%, about 10%, about 5%, about 2.5%, or about 1% of the surface area of the media is covered by the applied chemical(s). In various embodiments between: about 25% to about 75%, 30% to about 70%, 35% to about 65%, 40% to about 60%, 45% to about 65%, or about 45% to about 55% of the surface area of the media is covered by the applied chemical(s). In other embodiments, more than about 50%, about 50%, about 75%, about 85%, about 95%, about 100%, about 75% to about 100%, or about 90% about 100% of the surface area of the media is covered by the applied chemical(s). In yet other embodiments, more than 50%, more than 65%, more than 75%, more than 85%, more than 95%, or 100% of the surface area of the media is covered by the applied chemical(s). In certain embodiments, the chemical(s) is (are) allowed to remain on the surface of the media for at least about 1 minute, about 5 minutes, 10 minutes, about 15 minutes, about 20 minutes, about 30 minutes, about 45 minutes, or about 60 minutes (or a period of about 1 to about 5, 10, 15, 20, 30, 45, or 60 minutes) before a microorganism is applied. In certain embodiments, about 1 to about 203 bacterial colonies are streaked onto the media. In certain embodiments, the bacteria are recovered from freeze drying in liquid broth and then later diluted in additional liquid broth prior to being streaked onto the solid media. In certain embodiments, the microorganisms are streaked onto the media starting from the outside of the marked edge of plate, adjacent to where the chemical was applied, in decreasing concentric circles until the microorganisms have been streaked through the area where the chemical was applied. In any of the aforementioned embodiments, the bacteria or microorganisms are grown in a liquid medium (e.g. a broth) and then added to the surface of the solid media comprising a chemical (e.g., by streaking on the surface of the solid media).

In certain embodiments, the decreasing concentric circle streaking pattern does not simultaneously spread the microorganism and chemical. In certain embodiments, the chemical remains at the highest concentration at the center of where the chemical was applied and can diffuse in a circular pattern. In certain embodiments, the microorganism can survive everywhere that they are streaked, unless, for example, the chemical is an antimicrobial compound. In any of the aforementioned embodiments, the bacteria or microorganisms are grown in a liquid medium (e.g. a broth) and then added to the surface of the solid media comprising a chemical (e.g., by streaking on the surface of the solid media).

The disclosure provides the following non-limiting embodiments:

1. A kit comprising a first bacterium, a second bacterium, a third bacterium, and, optionally, a fourth bacterium, wherein
a) the first bacterium comprises the following combination of inactivated genes: glmS, thyA, cqsS, luxPQ, PHAP-1-like prophage, Kappa-like prophage, bioABFCD, and recA;
b) the second bacterium comprises the following combination of inactivated genes: glmS, thyA, cqsS, luxPQ, ØHAP-1-like prophage, Kappa-like prophage, bioABFCD, recA, and luxB;
c) the third bacterium comprises the following combination of inactivated genes: glmS, thyA, cqsS, luxPQ, @HAP-1-like prophage, Kappa-like prophage, bioABFCD, recA, and luxM; and
d) the fourth bacterium comprises the following combination of inactivated genes: glmS, thyA, cqsS, luxPQ, PHAP-1-like prophage, Kappa-like prophage, bioABFCD, recA, and a mutation in luxN that alters the activity of LuxN or inactivates luxN, preferably a mutation in luxN that causes a H471A missense mutation.

2. The kit of embodiment 1, wherein the kit further comprises Qstatin [1-(5-bromothiophene-2-sulfonyl)-1H-pyrazole].

3. The kit of embodiment 1 or 2, wherein the kit further comprises a LuxN inhibitor, a LuxR inhibitor, or a combination thereof.

4. The kit of embodiment 3, wherein the LuxN inhibitor is chlorolactone or C450-0730 and the LuxR inhibitor is 3-phenyl-1-(thiophen-2-ylsulfonyl)-1H-pyrazole (PTSP) or Qstatin.

5. The kit of embodiment 1, wherein the kit comprises Qstatin, chlorolactone, harveyi autoinducer-1 (HAI-1), said first bacterium, said second bacterium, said third bacterium, and said fourth bacterium and the fourth bacterium comprises a mutation that causes a H471A missense mutation in LuxN.

6. The kit of any one of embodiments 1-5, wherein said first, second, third, and, optional, fourth bacterium is Aliivibrio fischeri (formerly V. fischeri), Vibrio campbellii, Vibrio harveyi, Vibrio alginolyticus, Vibrio anguillarum, or Vibrio fluvialis, preferably Vibrio campbellii, Vibrio harveyi, Vibrio anguillarium, or Vibrio vulnificus.

7. The kit of embodiments 1-6, wherein said bacterium is Vibrio campbellii.

8. The kit of any one of embodiments 1-7, said kit further comprising culture medium, Petri dishes, and culture tubes.

9. a) culturing said first, second, third, and/or fourth bacterium in a liquid culture medium; and
b) plating each of said cultured bacteria on a separate non-overlapping area of a solid culture medium.

10. The method of embodiment 9, said method comprising:
a) plating said liquid cultured first bacterium and said liquid cultured second bacterium on a first solid culture medium in separate non-overlapping areas; and/or
b) plating said liquid cultured first bacterium and said liquid cultured third bacterium on a second solid culture medium in separate non-overlapping areas; and/or
c) plating said liquid cultured second bacterium and said liquid cultured third bacterium on a third solid culture medium in separate non-overlapping areas.

11. A recombinant bacterium comprising at least two inactivated genes, wherein a first inactivated gene is in the quorum sensing pathway of the bacterium and a second inactivated gene that confers attenuation or auxotrophy of the bacterium.

12. The recombinant bacterium of embodiment 11, wherein the bacterium belongs to the genus Vibrio.

13. The recombinant bacterium of any one of embodiments 11-12, wherein the bacterium is Aliivibrio fischeri (formerly V. fischeri), Vibrio campbellii, Vibrio harveyi, Vibrio alginolyticus, Vibrio anguillarum, or Vibrio fluvialis, preferably Vibrio campbellii, Vibrio harveyi, Vibrio anguillarium, or Vibrio vulnificus.

14. The recombinant bacterium of any one of embodiments 11-13, wherein the first inactivated gene is cqsS and the second inactivated gene comprises thyA.

15. The recombinant bacterium of any one of embodiments 11-14, said bacterium further comprises inactivation of:
a) glmS or orthologs thereof;
b) luxP and/or luxQ or orthologs thereof;
c) chromosomal regions encoding bacteriophage capsid and/or tail proteins, such as a ΦHAP-1-like bacteriophage region (SEQ ID NO: 8) or orthologs thereof;
d) chromosomal regions encoding bacteriophage capsid and/or tail proteins, such as a Kappa-like prophage region (SEQ ID NO: 9) or orthologs thereof;
e) recA or orthologs thereof;
f) bioABFCD, bioA, bioB, bioF, bioC, bioD, combinations of bioA, bioB, bioF, bioC, bioD or orthologs thereof;
g) hemA or orthologs thereof; or
h) any combination of a) through g).

16. The recombinant bacterium of any one of embodiments 11-15, wherein the bacterium further comprises inactivation of the luxN gene or missense mutations in the luxN gene resulting in the partial inactivation of the LuxN protein, such as a luxN gene encoding luxN having a missense mutation of H471A.

17. The recombinant bacterium of any one of embodiments 11-16, wherein the bacterium further comprises inactivation of the luxM gene.

18. The recombinant bacterium of any one of embodiments 11-17, wherein the bacterium further comprises inactivation of the luxB gene and/or luxA gene.

19. The recombinant bacterium of any one of embodiments 11-18, wherein said bacterium comprises a combination of inactivated genes introducing auxotrophy and a combination of inactivated quorum sensing genes, said combination of genes introducing auxotrophy being selected from:

Combinations of genes to introduce auxotrophy

| Combination | Combination | Combination | Combination |
|---|---|---|---|
| of 2 genes | of 3 genes | of 4 genes | of 5 genes |
| A and B | A, B, and C | A, B, C, and D | or A, B, C, D, and E |
| A and C | A, B, and D | A, B, C, and E | |
| A and D | A, B, and E | A, B, D, and E | |
| A and E | A, C, and D | A, C, D, and E | |
| B and C | A, C, and E | B, C, D, and E | |
| B and D | A, D, and E | | |
| B and E | B, C, and D | | |
| C and D | B, C, and E | | |
| C and E | B, D, and E | | |
| D and E | C, D, and E | | |

wherein A designates glmS, B designates thyA, C designates bioABFCD, D designates hemA, and E designates recA and bioABFCD designates each individual gene of the bioABFCD operon (bioA, bioB, bioF, bioC, and/or bioD) as well as the entire operon for the purposes of inactivation; and
said combination of inactivated quorum sensing genes being selected from:

Combinations of inactivated quorum sensing genes

| Combination | Combination | Combination | Combination |
|---|---|---|---|
| of 2 genes | of 3 genes | of 4 genes | of 5 genes |
| A and B | A, B, and C | A, B, C, and D | A, B, C, D, and E |
| A and C | A, B, and D | A, B, C, and E | A, B, C, D, and F |
| A and D | A, B, and E | A, B, C, and F | A, B, C, E, and F |
| A and E | A, B, and F | A, B, D, and E | A, B, D, E, and F |
| A and F | A, C, and D | A, B, D, and F | A, C, D, E, and F |
| B and C | A, C, and E | A, B, E, and F | B, C, D, E, and F |
| B and D | A, C, and F | A, C, D, and E | |
| B and E | A, D, and E | A, C, D, and F | |
| B and F | A, D, and F | A, C, E, and F | |
| C and D | A, E, and F | A, D, E, and F | |
| C and E | B, C, and D | B, C, D, and E | |
| C and F | B, C, and E | B, C, D, and F | |
| D and E | B, C, and F | B, C, E, and F | |
| D and F | B, D, and E | B, D, E, and F | |
| E and F | B, D, and F | C, D, E, and F | |
| | B, E, and F | | |
| | C, D, and E | | |
| | C, D, and F | | |
| | C, E, and F | | |
| | D, E, and F | | |

Six gene combination
or A, B, C, D, E, and F

wherein A designates cqsS, B designates luxPQ, C designates luxN, D designates luxM, E designates luxB, and F designates luxA and luxPQ designates the inactivation of luxP, luxQ or both luxP and luxQ.

20. The recombinant bacterium of any one of embodiments 11-19, wherein said bacterium comprises:
a) inactivation of glmS, thyA, cqsS, luxPQ, ØHAP-1-like prophage, Kappa-like prophage, bioABFCD, and recA;
b) inactivation of glmS, thyA, cqsS, luxPQ, @HAP-1-like prophage, Kappa-like prophage, bioABFCD, recA, and luxB;
c) inactivation of glmS, thyA, cqsS, luxPQ, HAP-1-like prophage, Kappa-like prophage, bioABFCD, recA, and luxM;
d) inactivation of glmS, thyA, cqsS, luxPQ, PHAP-1-like prophage, Kappa-like prophage, bioABFCD, recA, and luxN or a missense mutation in LuxN, such as a missense mutation in luxN that causes a H471A missense mutation; or
e) inactivation of glmS, thyA, bioABFCD, and recA and, optionally, a missense mutation in luxN that causes a H471A missense mutation; or
f) inactivation of glmS, thyA, bioABFCD, and recA and a missense mutation in luxN that causes a H471A missense mutation.

21. The recombinant bacterium of embodiment 20, wherein said bacterium comprises: inactivation of glmS, thyA, bioABFCD, and recA and a missense mutation in luxN that causes a H471A missense mutation.

22. A kit comprising at least one bacterium of any one of embodiments 11-21 and a ligand that binds to a quorum sensing receptor.

23. The kit of embodiment 22, wherein the ligand is harveyi autoinducer-1 (HAI-1).

24 The kit of any one of embodiments 22-23, further comprises a LuxN inhibitor, a LuxR inhibitor, or a combination thereof.

25. The kit of embodiment 24, wherein the LuxN inhibitor is chlorolactone or C450-0730 and the LuxR inhibitor is 3-phenyl-1-(thiophen-2-ylsulfonyl)-1H-pyrazole (PTSP) or QStatin.

26. The kit of any one of embodiments 22-25, comprising a first bacterium, a second bacterium, a third bacterium, and a fourth bacterium, wherein
a) the first bacterium comprises the following combination of inactivated genes: glmS, thyA, cqsS, luxPQ, ΦHAP-1-like prophage, Kappa-like prophage, bioABFCD, and recA;
b) the second bacterium comprises the following combination of inactivated genes: glmS, thyA, cqsS, luxPQ, ΦHAP-1-like prophage, Kappa-like prophage, bioABFCD, recA, and luxB;
c) the third bacterium comprises the following combination of inactivated genes: glmS, thyA, cqsS, luxPQ, ΦHAP-1-like prophage, Kappa-like prophage, bioABFCD, recA, and luxM; and
d) the fourth bacterium comprises the following combination of inactivated genes: glmS, thyA, cqsS, luxPQ, ΦHAP-1-like prophage, Kappa-like prophage, bioABFCD, recA, and luxN, preferably a mutation in luxN that causes a H471A missense mutation.

27. A pharmaceutical composition comprising the bacterium of any one of embodiments 11-21 and a pharmaceutically acceptable carrier.

28. A recombinant bacterium comprising one or more inactivated genes within the quorum sensing pathway of said bacterium, said one or more inactivated quorum sensing genes comprising cqsS, luxPQ, luxP, luxQ, luxM, luxN, luxA, luxB and combinations thereof.

29 The recombinant bacterium of embodiment 28, wherein the combination of inactivated quorum sensing genes is selected from one of the following combinations:

Combinations of inactivated quorum sensing genes

| Combination | Combination | Combination | Combination |
|---|---|---|---|
| of 2 genes | of 3 genes | of 4 genes | of 5 genes |
| A and B | A, B, and C | A, B, C, and D | A, B, C, D, and E |
| A and C | A, B, and D | A, B, C, and E | A, B, C, D, and F |
| A and D | A, B, and E | A, B, C, and F | A, B, C, E, and F |
| A and E | A, B, and F | A, B, D, and E | A, B, D, E, and F |
| A and F | A, C, and D | A, B, D, and F | A, C, D, E, and F |
| B and C | A, C, and E | A, B, E, and F | B, C, D, E, and F |
| B and D | A, C, and F | A, C, D, and E | |
| B and E | A, D, and E | A, C, D, and F | |
| B and F | A, D, and F | A, C, E, and F | |
| C and D | A, E, and F | A, D, E, and F | |
| C and E | B, C, and D | B, C, D, and E | |
| C and F | B, C, and E | B, C, D, and F | |
| D and E | B, C, and F | B, C, E, and F | |
| D and F | B, D, and E | B, D, E, and F | |
| E and F | B, D, and F | C, D, E, and F | |
| | B, E, and F | | |
| | C, D, and E | | |
| | C, D, and F | | |
| | C, E, and F | | |
| | D, E, and F | | |

Six gene combination
or A, B, C, D, E, and F

wherein A designates cqsS, B designates luxPQ, C designates luxN, D designates luxM, E designates luxB, and F designates luxA and luxPQ designates the inactivation of luxP, luxQ or both luxP and luxQ.

30. The recombinant bacterium of any one of embodiments 28 or 29, wherein the bacterium belongs to the genus Vibrio.

31. The recombinant bacterium of any one of embodiments 28, 29, or 30, wherein the recombinant bacterium is Aliivibrio fischeri (formerly V. fischeri), Vibrio campbellii, Vibrio harveyi, Vibrio alginolyticus, Vibrio anguillarum, or Vibrio fluvialis, preferably Vibrio campbellii, Vibrio harveyi, Vibrio anguillarium, or Vibrio vulnificus.

32. A plurality of bacterial cells comprising bacterial cells according to any one of embodiments 11-21 or 28-31, said plurality of bacterial cells comprising two or more strains of Vibrio bacterial cells.

33. The plurality of bacterial cells of embodiment 32, wherein said two or more strains of Vibrio are selected from Aliivibrio fischeri (formerly V. fischeri), Vibrio campbellii, Vibrio harveyi, Vibrio alginolyticus, Vibrio anguillarum, Vibrio fluvialis and combinations thereof.

34. A pharmaceutical composition comprising a recombinant bacterium of any one of embodiments 28-31 or a plurality of recombinant bacterial cells according to any one of embodiments 32-33.

35. A method for inducing protective immunity in a marine organism, the method comprising administering to the marine organism an effective amount of:
a) a pharmaceutical composition of embodiment 34;
b) a population of recombinant bacterial cells according to any one of embodiments 28-31 or a plurality of bacterial cells according to any one of embodiments 32-33;
c) a pharmaceutical composition of embodiment 27 and/or 34;
d) a population of recombinant bacterial cells of any one of embodiments 11-21;
e) a plurality of bacterial cells according to any one of embodiments 32-33; or
f) any combination of a) through e).

36. The method of embodiment 35, wherein the marine organism is a coral, oyster, prawn, shrimp, lobster, common snook, barramundi, turbot, milkfish, grouper, brown spotted grouper, tilapia, salmon, catfish, tuna, sole, sea perch, short sunfish, or seahorse.

37. A method of plating a microorganism on a growth medium, the method comprising:
i) applying a chemical to a location on a portion of a surface of the growth medium;
ii) applying the microorganism to the surface of the media adjacent to the location to which the chemical has been applied;
iii) spreading the microorganism around the location in circular pattern, whereby each revolution around the location to which the chemical has been applied has a decreased radius relative to the previous revolution around the location; and
iv) spreading the microorganism through the location to which the chemical has been applied in circular pattern, whereby each revolution through the location has a decreased radius relative to the previous revolution around the location to which the chemical has been applied.

38. The method of embodiment 37, wherein the portion of the surface of the growth medium to which a chemical is applied is less than about 75%, less than about 50%, or less than about 25% of the surface area of the growth medium.

39. The method of any one of embodiment 37 or 38, wherein the chemical is in liquid form and step ii) is performed at least about 1 minute after step i).

40. The method of any one of embodiments 37-39, wherein said microorganism is a recombinant bacterium of any one of embodiments 11-21 or a bacterial cell from the kit of any one of embodiments 22-26.

41. A method of plating a microorganism on a growth medium, the method comprising:
i) applying a chemical to a location on a portion of a surface of the growth medium;
ii) applying the microorganism to the surface of the media adjacent to the location to which the chemical has been applied, said microorganism being a bacterial cell from the kit of any one of embodiments 1-10, a recombinant bacterium of any one of embodiments 11-21 or a bacterial cell from the kit of any one of embodiments 22-26;
iii) spreading or streaking the microorganism on the surface of the growth medium; and
iv) spreading or streaking the microorganism through the location to which the chemical has been applied.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting.

### Example 1—Competitive Binding of Receptor and Ligand

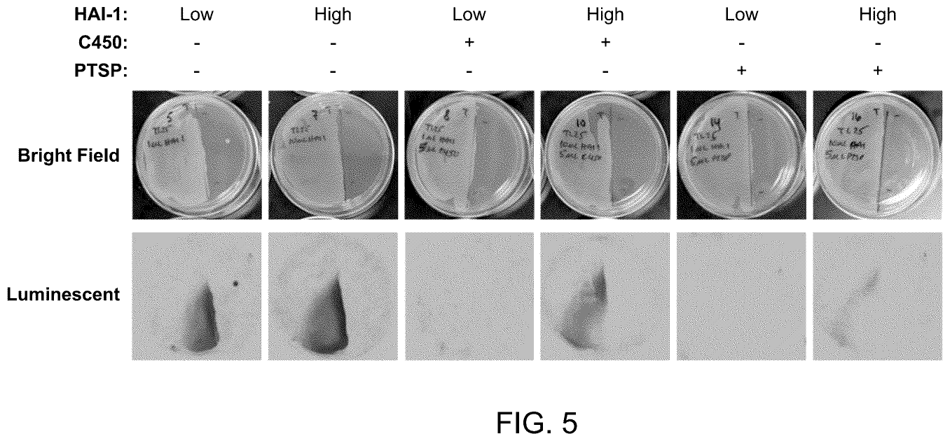

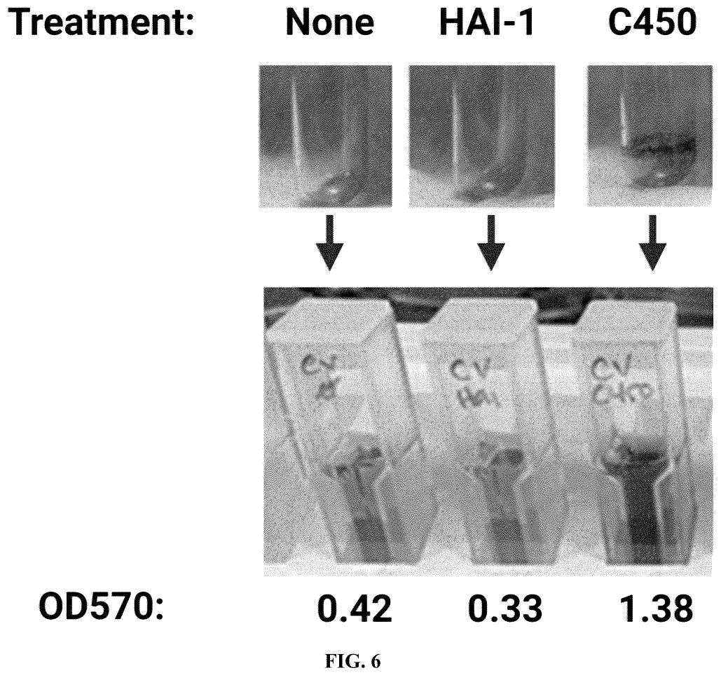

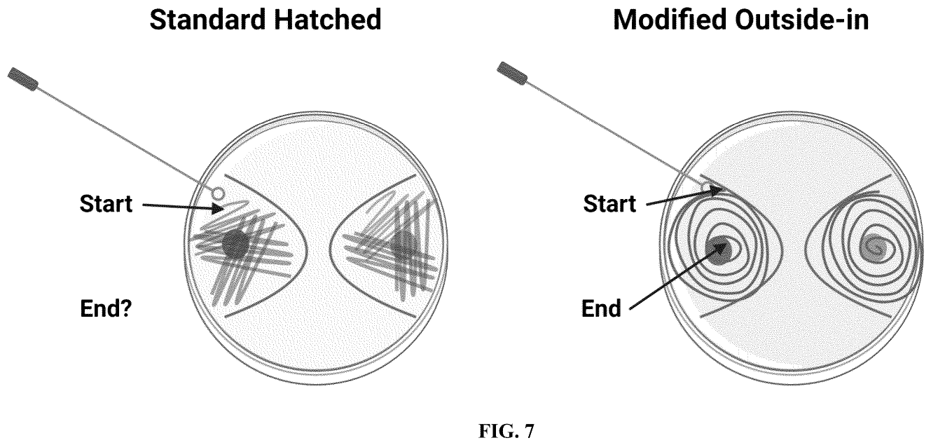

luxPQ-cqsS-luxM-triple mutant Vibrio campbellii are unable to glow without HAI-1 ligand present (FIG. 5). These plates were streaked with bacteria and subsequently treated with 50 nanomoles of upstream LuxN inhibitor (chlorolactone or C450-0730) or downstream LuxR inhibitor (QStatin). 10 or 100 nanomoles of HAI-1 synthetic ligand was added concurrently. Increasing amounts of ligand overcomes the upstream inhibitor yet the increasing amount of ligand does not overwhelm the downstream inhibitor.

### Example 2—Differences in the Formation of Biofilms

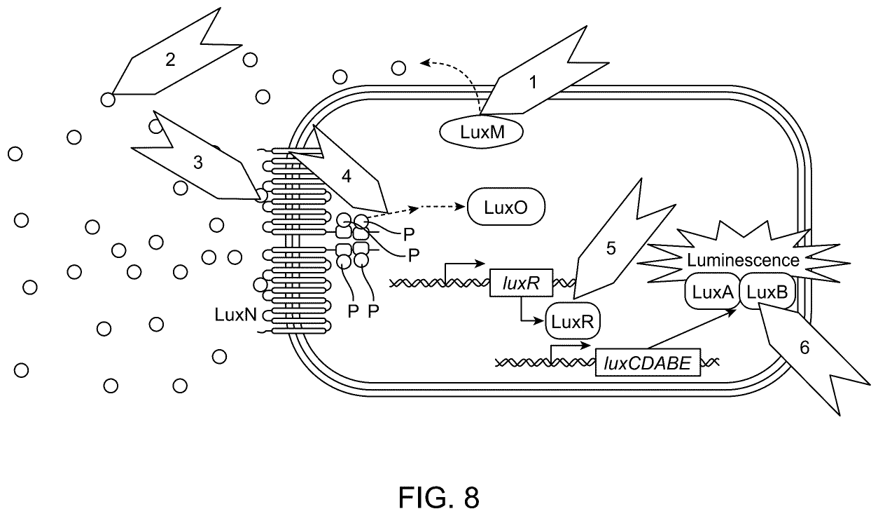

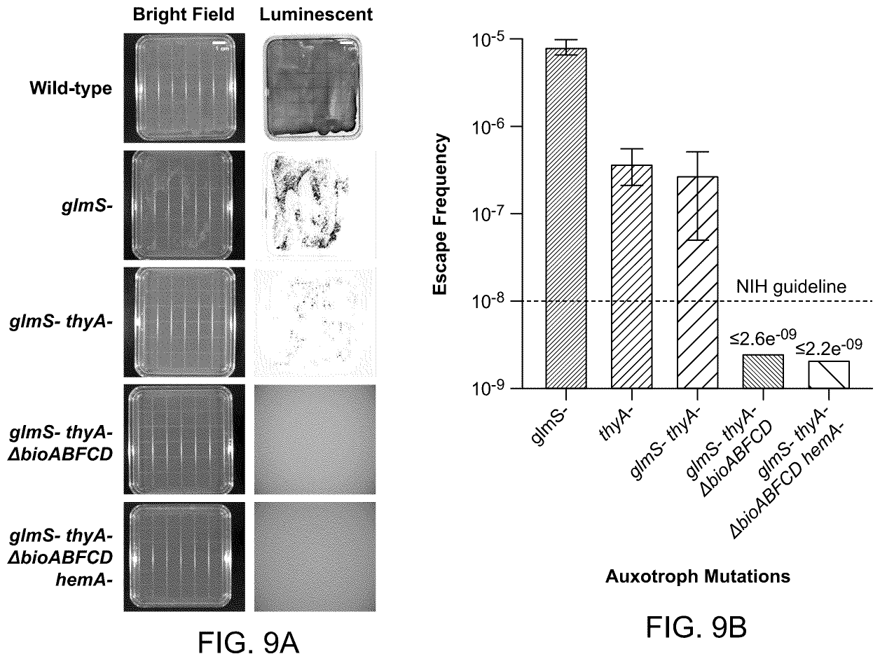

Without ligand activated signaling, Vibrio campbellii will not luminesce and will instead increase biofilm formation. cqsS-luxPQ-mutant cells treated with the LuxN inhibitor (chlorolactone or C450-0730) develop more biofilm than wild-type proxy liquid cultures, and wild-type proxy liquid cultures treated with HAI-1 synthetic ligand develop even less biofilm (FIG. 8). This can be demonstrated by staining the growth tubes from liquid cultures with crystal violet. Furthermore, the crystal violet stain can be dissolved in citric acid and then quantified with a spectrophotometer (FIG. 8, Table 3).

### Example 3—Observed Luminescence in Single Culture and Treated and Untreated Cocultures of Strains

When cultured singly, wild-type proxy strain (WT) and luxN-H471A (N-H471A) V. campbellii are able to luminesce brightly enough to be visible in darkened environments while luxB- and luxM-strains of bacteria show no to very minimal luminesce, respectively (FIG. 11). When luxM-strain is cocultured with WT, luxB-, or luxN-H471A strains, the regions of luxM-growth adjacent to the neighboring co-plated strain will demonstrate luminescence comparable to WT levels of luminescence. If the solid agar growth medium is pretreated with QStatin or PTSP prior to coculturing of strains, WT, luxM-, and luxN-H471A V. campbellii strains that would typically be showing luminescence will have decreased luminescence in the areas that are treated. luxB-strain will continue to show no luminescence. If the solid agar growth medium is pretreated with C450-0730 or Chlorolactone (Chlorolact.) prior to coculturing of strains, WT and luxM-V. campbellii strains that would typically be showing luminescence will have decreased luminescence in the areas that are treated. luxB-strain will continue to show no luminescence. luxN-H471A strain luminescence will not be affected by these two compounds. (FIG. 11: A, WT (top) & LuxB-(bottom); B, WT (top) & LuxM-(bottom), C, LuxB-(top) & LuxM-(bottom); D, LuxN-H471A (top) & LuxM-(bottom); and E&F, LuxM-(top) & LuxM-(bottom)). In preferred embodiments, single untreated, cocultured untreated, QStatin-treated cocultured, and Chlorolactone-treated cocultured conditions are utilized to highlight the function of strains and additive treatments.

TABLE 1
Combinations of genes to introduce auxotrophy

| Combination | Combination | Combination | Combination |
|---|---|---|---|
| of 2 genes | of 3 genes | of 4 genes | of 5 genes |
| A, B | A, B, C | A, B, C, D | A, B, C, D, E |
| A, C | A, B, D | A, B, C, E | |
| A, D | A, B, E | A, B, D, E | |
| A, E | A, C, D | A, C, D, E | |
| B, C | A, C, E | B, C, D, E | |
| B, D | A, D, E | | |
| B, E | B, C, D | | |
| C, D | B, C, E | | |
| C, E | B, D, E | | |
| D, E | C, D, E | | |

A designates glmS
B designates thyA
C designates bioABFCD
D designates hemA
E designates recA

TABLE 2
Combinations of quorum sensing gene modifications/mutations

| Combination | Combination | Combination | Combination |
|---|---|---|---|
| of 2 genes | of 3 genes | of 4 genes | of 5 genes |
| A, B | A, B, C | A, B, C, D | A, B, C, D, E |
| A, C | A, B, D | A, B, C, E | A, B, C, D, F |
| A, D | A, B, E | A, B, C, F | A, B, C, E, F |
| A, E | A, B, F | A, B, D, E | A, B, D, E, F |
| A, F | A, C, D | A, B, D, F | A, C, D, E, F |
| B, C | A, C, E | A, B, E, F | B, C, D, E, F |
| B, D | A, C, F | A, C, D, E | |
| B, E | A, D, E | A, C, D, F | |
| B, F | A, D, F | A, C, E, F | |
| C, D | A, E, F | A, D, E, F | |
| C, E | B, C, D | B, C, D, E | |
| C, F | B, C, E | B, C, D, F | |
| D, E | B, C, F | B, C, E, F | |
| D, F | B, D, E | B, D, E, F | |
| E, F | B, D, F | C, D, E, F | |
| | B, E, F | | |
| | C, D, E | | |
| | C, D, F | | |
| | C, E, F | | |
| | D, E, F | | |

Six gene combination
A, B, C, D, E, F
A designates cqsS
B designates luxPQ
C designates luxN
D designates luxM
E designates luxB
F designates luxA

TABLE 3
Biofilm Identification Using a Spectrophotometer

| Sample | | Crystal Violet | % Staining Compared |
|---|---|---|---|
| No | | OD570 | to No Treatment |
| 1 | No Treatment | 0.4212 | — |
| 2 | 10 μM HAI-1 | 0.3354 | 79% |
| 3 | 10 μM C450-0730 | 1.3839 | 328% |

TABLE 4
Exemplary strains for inclusion in kits

| Strain Name | Signaling Pathway Function | Strain Genotype |
|---|---|---|
| Wild-type | Intact signaling pathway; does | Vibrio campbellii glmS- thyA- cqsS- |
| proxy | not luminesce in low cell density | luxPQ- ΔHap9427 ΔKap10060 |
| (WT) | and luminesces at high cell density. | ΔbioABFCD recA- |
| luxB- | Strain cannot produce LuxB | Vibrio campbellii glmS- thyA- cqsS- |
| | protein; never luminesces. | luxPQ- ΔHap9427 ΔKap10060 |
| | | ΔbioABFCD recA- luxB- |
| luxM- | Strain cannot produce LuxM protein; | Vibrio campbellii glmS- thyA- cgsS- |
| | does not luminesce in low cell density | luxPQ- ΔHap9427 ΔKap10060 |
| | and does not luminesce at high cell | ΔbioABFCD recA- luxM- |
| | density. Able to luminesce in presence | |
| | of alternative source of ligand. | |
| luxN-H471A | Strain expresses missense | Vibrio campbellii glmS- thyA- cqsS- |
| | mutant of LuxN receptor; | luxPQ- ΔHap9427 ΔKap10060 |
| | luminesces at low and high cell | ΔbioABFCD recA- luxN-H471A |
| | density. | |

TABLE 5

| | | 5′ Sequence/Edited Region OR |
|---|---|---|
| | | Deleted Region (not shown)/3′ |
| Genotype | Notes | Sequence |
| glmS- | Gene encoding Glucosamine-6- | ATGTGTGGAATCGTAGGTG// |
| | phosphate synthase is removed. | AGCGGTAACCGTAGAGTAA |
| | Strain requires supplemental D- | (SEQ ID NOS: 21 and 22, |
| | glucosamine to survive. | respectively) |
| thyA- | Gene encoding thymidylate synthase | GTGAAACAGTATTTA// |
| | is removed. Strain requires | CCTTTCTCAGTTTAA |
| | supplemental thymidine to survive. | (SEQ ID NOS: 23 and 24, |
| | | respectively) |
| cqsS- | Quorum sensing receptor gene | GTGCTAGGCTCAGTAGATA// |
| | knockout. Strain loses ability to | TTCGGTACCTATTATTGCGC |
| | sense CAI-1 through CqsS. | ATACTGGTGATAGCTCACCG |
| | | ATAACACTGGACAAGATTG |
| | | GTTCATCTGGCATGTCGGA |
| | | TTTCATCGTTAAACCTGCG |
| | | GATAAGAACAGGTTGTTC |
| | | GACAAGATTGCGAACTGG |
| | | ATTTAG (SEQ ID NOs: 25 and 26, |
| | | respectively) |
| luxPQ- | Quorum sensing receptor gene | ATGAAGAAAGCG// |
| | knockout. Strain loses ability to | CGGTTTAATGGCA |
| | sense AI-2 through LuxPQ. | GTGATAAGT (SEQ ID NOs: 27 |
| | | and 28, respectively) |
| ΔHap9427 | ΦHAP-1-like prophage region | ATGCACATCAAACACCGGAG |
| | removed to reduce the ability of | TCAGCCTTGCCATATTTGGCA |
| | bacteria to produce functional phage. | GCAGACCGA//TAGGGTATGTG |
| | | CTCCCCTTACCAACTAATTCTT |
| | | CTTTAGTTCATTACCGC (SEQ |
| | | ID NOs: 29 and 30, |
| | | respectively) |
| ΔKap10060 | Kappa-like prophage region removed | CAACAAGGTAGCCAAGGCAA |
| | to reduce the ability of bacteria to | CCAGAGCCGATAGCCAAGTT |
| | produce functional phage. | GGGTCCATCG//AGCACCATTA |
| | | ACCTTCAGCAATCCCCTTTAT |
| | | TACTAAAGGGCTAACGGCT |
| | | (SEQ ID NOs: 31 and 32, |
| | | respectively) |
| ΔbioABFCD | Essential gene family knockout. | GTTTTTATTTTTTAAGTGTTC |
| | Deletion of biotin pathways genes at | TTCAAATTTAATGAGCGCAT |
| | the bioABFCD operon. Strain | TAAGCGAAG//AAACGCGTA |
| | requires supplemental biotin to | AAGGCTTCGCGATTCGATAA |
| | survive. | ATACAAAAAGGGCTGCGCAAT |
| | | (SEQ ID NOs: 33 and 34, |
| | | respectively) |
| hemA- | Essential gene knockout. Encodes | ATGTCCTTGCTTGCTATTGGA// |
| | hemA, a critical component of heme | GATGATCTGTAA (SEQ ID NOS: |
| | synthesis pathway. Strain requires | 35 and 36, respectively) |
| | supplemental 5-aminolevulinic acid | |
| | (ALA) to survive. | |
| luxM- | Quorum sensing ligand synthase | ATGAAATTAATGTTG// |
| | knockout. Targeted cells lose the | ACGTGCTGTTCGAGCG |
| | ability to synthesize HAI-1 ligand | AGCAGAAAACGCGGTT |
| | molecule. Cells will remain dark | CGTTAGAAAGGGATGA |
| | until exposed to source of HAI-1. | GCATGTTTGA (SEQ ID NOs: 37 |
| | | and 38, respectively) |
| luxB- | Quorum sensing luminescence | ATGAAATTTGGATTATTCT// |
| | protein knockout. Strain loses the | CGTCAAATACCACTCGTAA |
| | ability to produce LuxB enzyme. | (SEQ ID NOs: 39 and 40, |
| | Cells unable to luminesce. | respectively) |
| luxN H471A | Quorum sensing receptor LuxN gene | CGCGCACTAGCTAACTCTA |
| | missense mutation. LuxN unable to | TTGCT/CAC/GAAATGCGTA |
| | autophosphorylate disrupting LuxN | ACCCTCTTGCTCAAGT (SEQ ID |
| | dependent phosphorelay. | NO: 41) |
| recA- | DNA damage response and repair | AAAGTGATGGACGAG// |
| | gene knockout. Cells lacks RecA | GAAGAGTTTTAATCTTCT (SEQ |
| | bacterial recombinase resulting in | ID NOs: 42 and 43, |
| | decreased recombination-based | respectively) |
| | DNA repair. | |

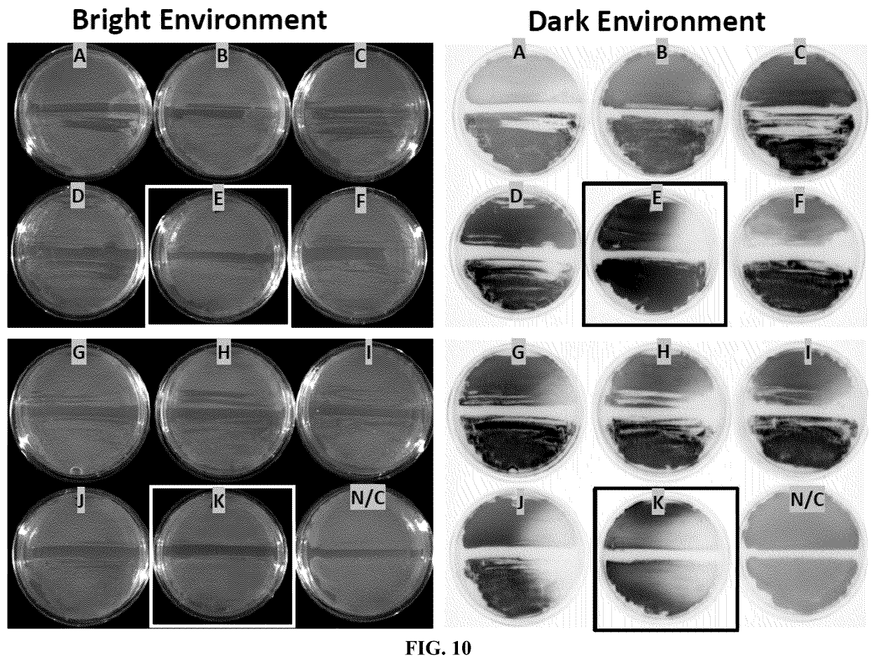

TABLE 6
Summary of the Results of FIG. 10

| | | Reduction of |
|---|---|---|
| | | Luminescence at |
| | Chemical Name | 22-24 Hours |
| A | C450-0730 | ++ |
| B | C646-0078 | − |
| C | 3578-0898 | − |
| D | 4248-0174 | − |
| E | Chlorolactone | +++ |
| F | N-Dodecanoyl-L-homoserine lactone | + |
| G | N-Decanoyl-L-homoserine lactone | − |
| H | N-Octanoy1-L-homoserine lactone | − |
| I | N-(3-Oxododecanoyl)-L-homoserine lactone | − |
| J | QStatin | +++ |
| K | PTSP | +++ |
| N/C | N/A | − |

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated within the scope of the invention without limitation thereto.

We claim:

1. A kit comprising a first bacterium, a second bacterium, a third bacterium, and, optionally, a fourth bacterium, wherein
a) the first bacterium comprises the following combination of inactivated genes: glmS, thyA, cqsS, luxPQ, PHAP-1-like prophage, Kappa-like prophage, bioABFCD, and recA;
b) the second bacterium comprises the following combination of inactivated genes: glmS, thyA, cqsS, luxPQ, PHAP-1-like prophage, Kappa-like prophage, bioABFCD, recA, and luxB;
c) the third bacterium comprises the following combination of inactivated genes: glmS, thyA, cqsS, luxPQ, PHAP-1-like prophage, Kappa-like prophage, bioABFCD, recA, and luxM; and
d) the fourth bacterium comprises the following combination of inactivated genes: glmS, thyA, cqsS, luxPQ, PHAP-1-like prophage, Kappa-like prophage, bioABFCD, recA, and a mutation in luxN that alters the activity of LuxN or inactivates luxN.

2. The kit of claim 1, wherein the kit further comprises QStatin [1-(5-bromothiophene-2-sulfonyl)-1H-pyrazole].

3. The kit of claim 1, wherein the kit further comprises a LuxN inhibitor, a LuxR inhibitor, or a combination thereof.

4. The kit of claim 3, wherein the LuxN inhibitor is chlorolactone or C450-0730 and the LuxR inhibitor is 3-phenyl-1-(thiophen-2-ylsulfonyl)-1H-pyrazole (PTSP) or QStatin.

5. The kit of claim 1, wherein the kit comprises Qstatin, chlorolactone, harveyi autoinducer-1 (HAI-1), said first bacterium, said second bacterium, said third bacterium, and said fourth bacterium and the fourth bacterium comprises a mutation that causes a H471A missense mutation in LuxN.

6. The kit of claim 1, wherein said first, second, third, and, optional, fourth bacterium is Aliivibrio fischeri (formerly V. fischeri), Vibrio campbellii, Vibrio harveyi, Vibrio alginolyticus, Vibrio anguillarum, or Vibrio fluvialis, preferably Vibrio campbellii, Vibrio harveyi, Vibrio anguillarium, or Vibrio vulnificus.

7. The kit of claim 6, wherein said bacterium is Vibrio campbellii.

8. The kit of claim 1, said kit further comprising culture medium, Petri dishes, and culture tubes.

9. A method of plating a bacterium of the kit of claim 1, said method comprising:
a) culturing said first, second, third, and/or fourth bacterium in a liquid culture medium; and
b) plating each of said cultured bacteria on a separate non-overlapping area of a solid culture medium.

10. The method of claim 9, said method comprising:
a) plating said liquid cultured first bacterium and said liquid cultured second bacterium on a first solid culture medium in separate non-overlapping areas; and/or
b) plating said liquid cultured first bacterium and said liquid cultured third bacterium on a second solid culture medium in separate non-overlapping areas; and/or
c) plating said liquid cultured second bacterium and said liquid cultured third bacterium on a third solid culture medium in separate non-overlapping areas.

11. A recombinant bacterium comprising at least two inactivated genes, wherein a first inactivated gene is in the quorum sensing pathway of the bacterium and a second inactivated gene that confers attenuation or auxotrophy of the bacterium.

12. The recombinant bacterium of claim 11, wherein said bacterium comprises a combination of inactivated genes introducing auxotrophy and a combination of inactivated quorum sensing genes, said combination of genes introducing auxotrophy being selected from:
Combinations of genes to introduce auxotrophy

| Combination | Combination | Combination | Combination |
|---|---|---|---|
| of 2 genes | of 3 genes | of 4 genes | of 5 genes |
| A and B | A, B, and C | A, B, C, and D | or A, B, C, D, and E |
| A and C | A, B, and D | A, B, C, and E | |
| A and D | A, B, and E | A, B, D, and E | |
| A and E | A, C, and D | A, C, D, and E | |
| B and C | A, C, and E | B, C, D, and E | |
| B and D | A, D, and E | | |
| B and E | B, C, and D | | |
| C and D | B, C, and E | | |
| C and E | B, D, and E | | |
| D and E | C, D, and E | | |

wherein A designates glmS, B designates thyA, C designates bioABFCD, D designates hemA, and E designates recA and bioABFCD designates each individual gene of the bioABFCD operon (bioA, bioB, bioF, bioC, and/or bioD) as well as the entire operon for the purposes of inactivation; and
said combination of inactivated quorum sensing genes being selected from:
Combinations of inactivated quorum sensing genes

| Combination | Combination | Combination | Combination |
|---|---|---|---|
| of 2 genes | of 3 genes | of 4 genes | of 5 genes |
| A and B | A, B, and C | A, B, C, and D | A, B, C, D, and E |
| A and C | A, B, and D | A, B, C, and E | A, B, C, D, and F |
| A and D | A, B, and E | A, B, C, and F | A, B, C, E, and F |
| A and E | A, B, and F | A, B, D, and E | A, B, D, E, and F |
| A and F | A, C, and D | A, B, D, and F | A, C, D, E, and F |
| B and C | A, C, and E | A, B, E, and F | B, C, D, E, and F |
| B and D | A, C, and F | A, C, D, and E | |
| B and E | A, D, and E | A, C, D, and F | |
| B and F | A, D, and F | A, C, E, and F | |
| C and D | A, E, and F | A, D, E, and F | |
| C and E | B, C, and D | B, C, D, and E | |
| C and F | B, C, and E | B, C, D, and F | |
| D and E | B, C, and F | B, C, E, and F | |
| D and F | B, D, and E | B, D, E, and F | |
| E and F | B, D, and F | C, D, E, and F | |
| | B, E, and F | | |
| | C, D, and E | | |
| | C, D, and F | | |
| | C, E, and F | | |
| | D, E, and F | | |

Six gene combination
or A, B, C, D, E, and F
wherein A designates cqsS, B designates luxPQ, C designates luxN, D designates luxM, E designates luxB, and F designates luxA and luxPQ designates the inactivation of luxP, luxQ or both luxP and luxQ.

13. The recombinant bacterium of claim 11, wherein said bacterium comprises:
a) inactivation of glmS, thyA, cqsS, luxPQ, PHAP-1-like prophage, Kappa-like prophage, bioABFCD, and recA;
b) inactivation of glmS, thyA, cqsS, luxPQ, ΦHAP-1-like prophage, Kappa-like prophage, bioABFCD, recA, and luxB;
c) inactivation of glmS, thyA, cqsS, luxPQ, PHAP-1-like prophage, Kappa-like prophage, bioABFCD, recA, and luxM;
d) inactivation of glmS, thyA, cqsS, luxPQ, PHAP-1-like prophage, Kappa-like prophage, bioABFCD, recA, and luxN or a missense mutation in LuxN, such as a missense mutation in luxN that causes a H471A missense mutation; or
e) inactivation of glmS, thyA, bioABFCD, and recA and, optionally, a missense mutation in luxN that causes a H471A missense mutation; or
f) inactivation of glmS, thyA, bioABFCD, and recA and a missense mutation in luxN that causes a H471A missense mutation.

14. A kit comprising at least one bacterium of claim 11 and a ligand that binds to a quorum sensing receptor.

15. A pharmaceutical composition comprising the bacterium of claim 11 and a pharmaceutically acceptable carrier.

16. A recombinant bacterium comprising one or more inactivated genes within the quorum sensing pathway of said bacterium, said one or more inactivated quorum sensing genes comprising cqsS, luxPQ, luxP, luxQ, luxM, luxN, luxA, luxB and combinations thereof or a plurality of said bacterial cells.

17. A pharmaceutical composition comprising a recombinant bacterium of claim 16 or a plurality of said bacterial cells.

18. A method for inducing protective immunity in a marine organism, the method comprising administering to the marine organism an effective amount of a recombinant bacterium or claim 16 or a plurality of said recombinant bacterial cells, or a pharmaceutical compositions comprising said recombinant bacterium or said plurality of said recombinant bacterial cells.

19. A method of plating a microorganism on a growth medium, the method comprising:
i) applying a chemical to a location on a portion of a surface of the growth medium;
ii) applying the microorganism to the surface of the media adjacent to the location to which the chemical has been applied, said microorganism being a bacterial cell from the kit of claim 1;
iii) spreading the microorganism around the location in circular pattern, whereby each revolution around the location to which the chemical has been applied has a decreased radius relative to the previous revolution around the location; and
iv) spreading the microorganism through the location to which the chemical has been applied in circular pattern, whereby each revolution through the location has a decreased radius relative to the previous revolution around the location to which the chemical has been applied.

20. A method of plating a microorganism on a growth medium, the method comprising:
i) applying a chemical to a location on a portion of a surface of the growth medium;
ii) applying the microorganism to the surface of the media adjacent to the location to which the chemical has been applied, said microorganism being a bacterial cell from the kit of claim 1;
iii) spreading or streaking the microorganism on the surface of the growth medium; and
iv) spreading or streaking the microorganism through the location to which the chemical has been applied.
